(19) United States
(12) Patent Application Publication
(10) Pub. No.: US 2026/0109678 A1
(43) Pub. Date: Apr. 23, 2026

(54) SUBSTITUTED PHENOTHIAZINES AS FERROPTOSIS INHIBITORS

(71) Applicant: Universiteit Antwerpen, Antwerpen (BE)

(72) Inventors: Koen AUGUSTYNS, Wilrijk (BE); Pieter VAN DER VEKEN, Wilrijk (BE); Tom VANDEN BERGHE, Wilrijk (BE); Behrouz HASSANNIA, Wilrijk (BE); Caroline LANTHIER, Wilrijk (BE); Camilla SCARPELLINI, Wilrijk (BE); Greta KLEJBOROWSKA, Wilrijk (BE)

(73) Assignee: Universiteit Antwerpen, Antwerpen (BE)

(21) Appl. No.: 19/113,145

(22) PCT Filed: Sep. 21, 2023
(86) PCT No.: PCT/EP2023/076090

§ 371 Date: Mar. 19, 2025

(30) Foreign Application Priority Data
Sep. 21, 2022 (EP) 22196811.8

(51) Int. Cl. C07D 279/20; A61K 31/5415

(52) U.S. Cl. CPC C07D 279/20; A61K 31/5415

(57) ABSTRACT

The present invention relates to a compound of formula (I) or a stereoisomer, or tautomer, wherein R1, R2, R3, and R4 have the same meaning as that defined in the claims and the description. The present invention also relates to the use of such compounds for the prevention and/or treatment of a disease associated with ferroptosis and/or oxytosis such as liver disease, chronic kidney disease, ocular surface diseases, wound healing, multiple organ dysfunction syndrome, neurological disease, acute renal failure, ischemia-reperfusion injury, sepsis, prevention of transplant rejection, and iron metabolism-related disease. The present invention also provides pharmaceutical compositions comprising such compounds, as well as the use of the compounds as a medicament.

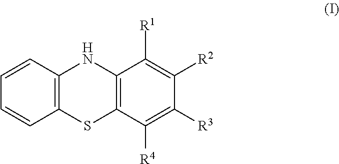

### FIELD OF THE INVENTION

The present invention relates to phenothiazine derivative compounds, or pharmaceutically acceptable salts thereof, as defined herein, that are useful for inhibiting undesired cell death occurring in diseases associated with ferroptosis and/or oxytosis such as neurodegenerative diseases, ischemia-reperfusion injury and iron toxicity. The invention also provides compositions containing a pharmaceutically acceptable carrier and one or more compounds from the invention.

### BACKGROUND OF THE INVENTION

Cell death is crucial for normal development, homeostasis, and the prevention of hyperproliferative diseases such as cancer. It was once thought that almost all regulated cell death in mammalian cells resulted from the activation of caspase-dependent apoptosis. This view has been challenged by the discovery of several regulated non-apoptotic cell death pathways activated in specific disease states. Regulated necrosis is defined as a genetically controlled cell death process that eventually results in cellular leakage, and is morphologically characterized by cytoplasmic granulation, as well as organelle and/or cellular swelling.

Ferroptosis is one recognized form of regulated necrosis and its hallmark is the production of iron-dependent lipophilic reactive oxygen species (ROS). Ferroptosis is partly mediated through inhibiting the system Xc⁻ Cys/Glu antiporter, which allows the exchange of extracellular L-Cys and intracellular L-Glu across the plasma membrane. Ferroptosis involves metabolic dysfunction that results in the production of both cytosolic and phospholipid ROS. It is believed that ROS generated by Fenton-type reactions (dependent on the availability of catalytic ferrous iron), rather than the mitochondrial electron transport chain are the main drivers of ferroptosis. Glutathione (GSH) peroxidase 4 (GPX4) is a crucial inhibitor of ferroptosis, and its activity relies on GSH levels. Therefore, GSH depletion typically leads to loss-of-function of GPX4, resulting in ROS-mediated phospholipid peroxidation. In addition to ferroptosis, glutamine- and oxidative stress induced cell death are inhibited by iron chelation. In line with this, iron-dependent neuronal cell death is blocked by metal protein-attenuating compounds (e.g. clioquinol) and iron chelators (e.g. desferroxamine), which are being explored for the treatment of neurodegenerative diseases. Another type of regulated necrosis is oxytosis which is also induced when the Xc⁻ Cys/Glu antiporter is inhibited through an excess of the neurotransmitter glutamine, the latter process is often designated as excitotoxicity in neuronal cells. Because of the clear mechanistic overlaps between oxytosis and ferroptosis it is likely that the use of modulators of ferroptosis in disease will target the same disease processes which are associated with oxytosis. Disease processes where undesired ferroptosis and/or oxytosis occur are typically disorders where an oxidative stress factor is involved such as for example in several neurodegenerative diseases, liver-, cardiac- and kidney-ischaemia—reperfusion injury, stroke, sepsis, diabetes and epilepsy. Oxidative stress due to iron overload is for example highly relevant in organs accumulating iron such as the brain, kidney and liver. Several compounds have been described in the art which are able to inhibit ferroptosis such as for example WO2013/152039, Skouta R et al (2014) J. Am. Chem. Soc. 136, 4551-4556). The prior art highlights the importance of the ethyl-ester in the maintenance of the potency of the first-in-class compound ferroptosis inhibitor molecule (designated as Ferrostatin-1) and there have been suggestions for chain modifications of the ester for generating improved molecules. Indeed, the latter reference also teaches that esters modified to amides and sulfonamides at the same position have a lower EC50. It has also been suggested that improved pharmacokinetic variants of Ferrostatin-1 could be ester analogues. It would be desirable to generate additional compounds that can inhibit or reduce ferroptosis, in particular compounds with an improved physicochemical and/or pharmacokinetic profile.

There is a need for small molecules that use ferroptosis and/or oxytosis as their target, have strong selectivity, have properties that are amenable to develop them as pharmaceuticals, such as high solubility, and have significant efficacy for the treatment of the ferroptosis and/or oxytosis related diseases.

### SUMMARY OF THE INVENTION

The present invention is based on the unexpected finding that at least one of the above-mentioned objectives can be attained by small molecules.

The present invention provides compounds which have surprisingly found to be potent inhibitors of ferroptosis and/or oxytosis, while also displaying moderate to high solubility; in addition, the compounds of the invention show moderate to high CNS MPO scores and low in vitro cytotoxicity. In view thereof, these compounds can be used to treat diseases where an excess of ferroptosis and/or oxytosis occurs.

A first aspect of the present invention provides a compound of formula (I) or a stereoisomer, or tautomer thereof,

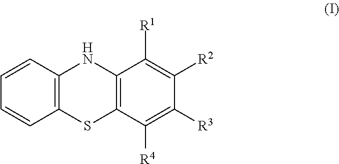

wherein,
R¹ is selected from the group consisting of hydrogen, amino, —NR⁶R⁷, halo, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z¹;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, cycloalkyl, aryl, heterocyclyl, and heteroaryl; wherein said cycloalkyl, aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1a};
each R⁸, R⁹, R¹², R¹³, R¹⁴ and R¹⁵ is selected from the group consisting of hydrogen, halogen, hydroxyl, amino, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1b};
each R¹⁰, R¹¹ and R¹⁶ is selected from the group consisting of heterocyclyl, —NR⁶R⁷, alkyl, cycloalkyl, aryl, arylalkyl, heterocyclyl, heterocyclylalkyl, heteroaryl, and heteroarylalkyl; wherein said heterocyclyl, alkyl, cycloalkyl, aryl, arylalkyl, heterocyclylalkyl, heteroaryl or heteroarylalkyl can be unsubstituted or substituted with one or more Z^{1c};
each R¹⁷ is independently selected from the group consisting of alkyl, aryl, cycloalkyl, arylalkyl, heterocyclyl, heteroaryl;
each R¹⁸ and R¹⁹ is independently selected from the group consisting of hydrogen, alkyl, aryl, cycloalkyl, arylalkyl, heterocyclyl, heteroaryl;
each Z¹, Z², Z³, Z⁴, Z^{1a}, and Z^{1b} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano,
amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; each Z^{1c} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano, amino, —NR¹⁷R¹⁸, —C(O)₂R¹⁸, —C(O)NR¹⁸R¹⁹, —S(O)R¹⁸, —S(O)₂R¹⁸, —S(O)₂NR¹⁸R¹⁹, nitro;
wherein when R¹ is

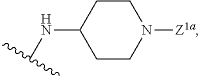

then R³ is not H, Cl or —C(O)R¹¹;
or a solvate, hydrate, pharmaceutically acceptable salt, or prodrug thereof;
with the proviso that said compound is not

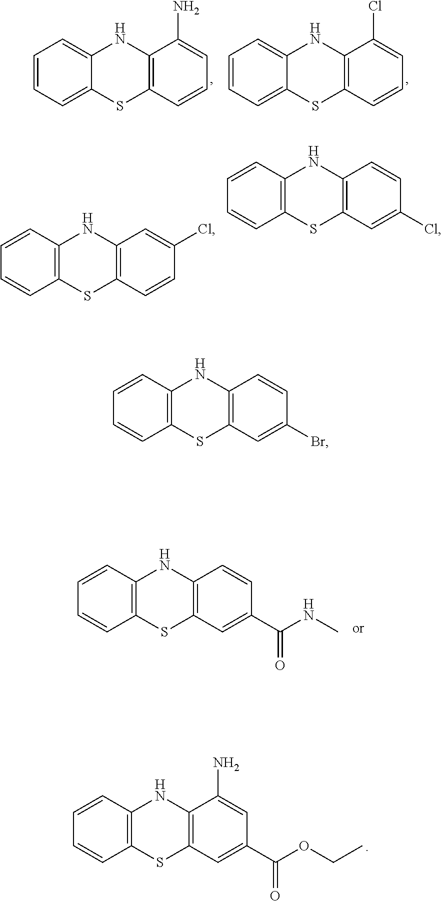

A related aspect of the present invention provides a compound of formula (I) or a stereoisomer, or tautomer thereof,

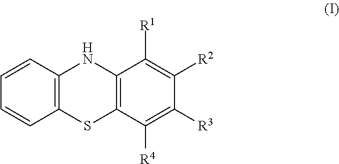

wherein,
R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z²;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, cycloalkyl, aryl, heterocyclyl, and heteroaryl; wherein said cycloalkyl, aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1a};
each R⁸, R⁹, R¹², R¹³, R¹⁴ and R¹⁵ is selected from the group consisting of hydrogen, halogen, hydroxyl, amino, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1b};
each R¹⁰, R¹¹ and R¹⁶ is selected from the group consisting of heterocyclyl, —NR⁶R⁷, alkyl, cycloalkyl, aryl, arylalkyl, heterocyclyl, heterocyclylalkyl, heteroaryl, and heteroarylalkyl; wherein said heterocyclyl, alkyl, cycloalkyl, aryl, arylalkyl, heterocyclylalkyl, heteroaryl or heteroarylalkyl can be unsubstituted or substituted with one or more Z^{1c};
each R¹⁷ is independently selected from the group consisting of, alkyl, aryl, cycloalkyl, arylalkyl, heterocyclyl, heteroaryl;
each R¹⁸ and R¹⁹ is independently selected from the group consisting of hydrogen, alkyl, aryl, cycloalkyl, arylalkyl, heterocyclyl, heteroaryl;
each Z¹, Z², Z³, Z⁴, Z^{1a}, Z^{1b}, and Z^{1c} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano,
amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; or a solvate, hydrate, pharmaceutically acceptable salt, or prodrug thereof;
with the proviso that said compound is not

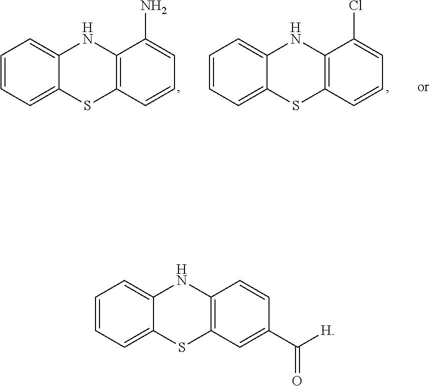

A second, related aspect of the present invention provides a pharmaceutical composition comprising a compound of formula (I) as described in the first aspect of the invention, and a pharmaceutically acceptable carrier.

A third aspect of the present invention provides a compound of formula (I) as described in the first aspect of the invention or a pharmaceutical composition as described in the second aspect of the invention, or a compound selected from the group consisting of

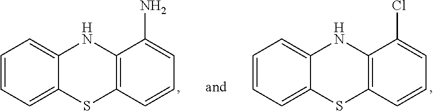 [SMILES: Nc1cccc2c1Nc1ccccc1S2 ; Clc1cccc2c1Nc1ccccc1S2]

for use as a medicament.

In some embodiments, the present invention also encompasses a compound of formula (I) as described in the first aspect of the invention or a pharmaceutical composition as described in the second aspect of the invention, or a compound selected from the group consisting of

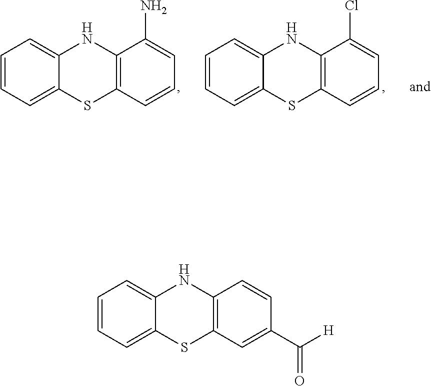

for use as a medicament.

According to a fourth, related aspect, the present invention also encompasses a compound according to the first aspect of the invention, or a pharmaceutical composition according to the second aspect of the invention, or a compound selected from the group consisting of

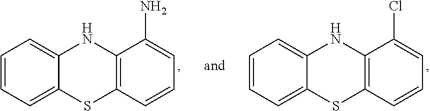 [SMILES: Nc1cccc2c1Nc1ccccc1S2 ; Clc1cccc2c1Nc1ccccc1S2]

for use in the prevention or treatment of a disease associated with ferroptosis and/or oxytosis.

In some embodiments, the present invention also encompasses a compound according to the first aspect of the invention, or a pharmaceutical composition according to the second aspect of the invention, or a compound selected from the group consisting of

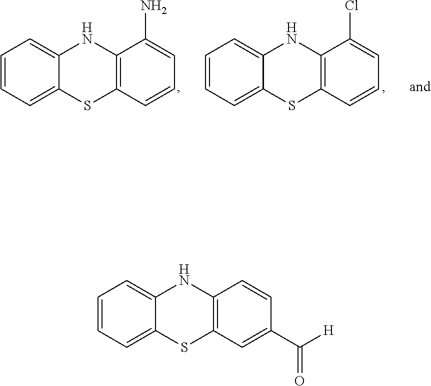

for use in the prevention or treatment of a disease associated with ferroptosis and/or oxytosis.

According to a fifth, related aspect, the present invention also encompasses a compound according to the first aspect of the invention or a or a pharmaceutical composition according to the second aspect of the invention, or a compound selected from the group consisting of

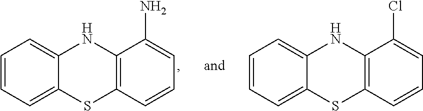 [SMILES: Nc1cccc2c1Nc1ccccc1S2 ; Clc1cccc2c1Nc1ccccc1S2]

for use in the prevention or treatment of liver disease, chronic kidney disease, ocular surface diseases, wound healing, multiple organ dysfunction syndrome, neurological disease, acute renal failure, ischemia-reperfusion injury, iron toxicity, sepsis, prevention of transplant rejection, and iron metabolism-related disease.

In some embodiments, the present invention also encompasses a compound according to the first aspect of the invention or a or a pharmaceutical composition according to the second aspect of the invention, or a compound selected from the group consisting of

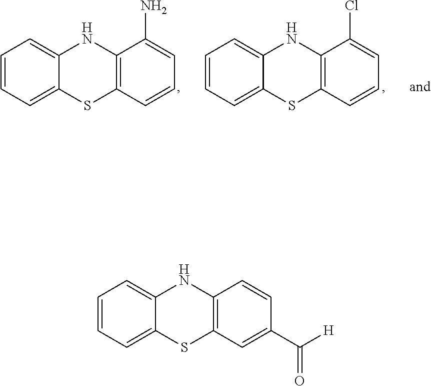

for use in the prevention or treatment of liver disease, chronic kidney disease, ocular surface diseases, wound healing, multiple organ dysfunction syndrome, neurological disease, acute renal failure, ischemia-reperfusion injury, iron toxicity, sepsis, prevention of transplant rejection, and iron metabolism-related disease.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The independent and dependent claims set out particular and preferred features of the invention. Features from the dependent claims may be combined with features of the independent or other dependent claims as appropriate.

### DETAILED DESCRIPTION OF THE INVENTION

Before the present invention is described, it is to be understood that this invention is not limited to particular processes, methods, and compounds described, as such processes, methods, and compounds may, of course, vary. It is also to be understood that the terminology used herein is not intended to be limiting, since the scope of the present invention will be limited only by the appended claims.

When describing the compounds and processes of the invention, the terms used are to be construed in accordance with the following definitions, unless a context dictates otherwise.

As used in the specification and the appended claims, the singular forms “a”, “an,” and “the” include both singular and plural referents unless the context clearly dictates otherwise. By way of example, “a compound” means one compound or more than one compound.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiments but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments combinations of features of different embodiments are meant to be within the scope of the invention, and form different embodiments as would be understood by those in the art. For example, in the following claims, any of the claimed embodiments can be used in any combination.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

When describing the present invention, the terms used are to be construed in accordance with the following definitions, unless a context dictates otherwise.

The terms described above and others used in the specification are well understood to those in the art.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture.

Where groups can be substituted, such groups may be substituted with one or more, and preferably one, two or three substituents. Preferred substituents may be selected from but not limited to, for example, the group comprising halo, hydroxyl, alkyl, alkoxy, trifluoromethyl, trifluoromethoxy, cycloalkyl, aryl, arylalkyl, heterocyclyl, heteroaryl, cyano, amino, nitro, carboxyl, and mono- or dialkylamino.

The term “halo” or “halogen” as a group or part of a group is generic for fluoro, chloro, bromo, iodo.

The term “hydroxyl” or “hydroxy” as used herein refers to the group —OH.

The term “cyano” as used herein refers to the group —C≡N.

The term “amino” as used herein refers to the —NH₂ group.

The term “nitro” as used herein refers to the —NO₂ group.

The term “carboxy” or “carboxyl” or “hydroxycarbonyl” as used herein refers to the group —CO₂H.

The term “aminocarbonyl” as used herein refers to the group —CONH₂.

The term “alkyl”, as a group or part of a group, refers to a hydrocarbyl group of formula —CₙH₂ₙ₊₁ wherein n is a number greater than or equal to 1. Alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, “C₁₋₆alkyl” includes all linear or branched alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers. For example, “C₁₋₅alkyl” includes all includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example “C₁₋₃alkyl” includes all linear or branched alkyl groups with between 1 and 3 carbon atoms, and thus includes methyl, ethyl, n-propyl, i-propyl.

When the term “alkyl” is used as a suffix following another term, as in “hydroxyalkyl,” this is intended to refer to an alkyl group, as defined above, being substituted with one or two (preferably one) substituent(s) selected from the other, specifically-named group, also as defined herein. The term “hydroxyalkyl” therefore refers to a —R^{a}—OH group wherein R^{a} is alkylene as defined herein.

The term “haloalkyl” as a group or part of a group, refers to an alkyl group having the meaning as defined above wherein one, two, or three hydrogen atoms are each replaced with a halogen as defined herein. Non-limiting examples of such haloalkyl groups include chloromethyl, 1-bromoethyl, fluoromethyl, difluoromethyl, trifluoromethyl, 1,1,1-trifluoroethyl, trichloromethyl, tribromomethyl, and the like.

The term “trifluoromethyl” as used herein refers to the group —CF₃.

The term “difluoromethyl” as used herein refers to the group —CHF₂.

The term “trifluoromethoxy” as used herein refers to the group —OCF₃.

The term “difluoromethoxy” as used herein refers to the group —OCHF₂.

The term “alkoxy” or “alkyloxy”, as a group or part of a group, refers to a group having the formula —OR^{b} wherein R^{b} is alkyl as defined herein above. Non-limiting examples of suitable alkoxy include methoxy, ethoxy, propoxy, isopropoxy, butoxy, isobutoxy, sec-butoxy, tert-butoxy, pentyloxy and hexyloxy.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₈ cycloalkyl”, a cyclic alkyl group comprising from 3 to 8 carbon atoms. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl, 1-adamantyl.

The term “cycloalkyloxy”, as a group or part of a group, refers to a group having the formula —OR^{f} wherein R^{f} is cycloalkyl as defined herein above.

The term “aryl”, as a group or part of a group, refers to a polyunsaturated, aromatic hydrocarbyl group having a single ring (i.e. phenyl) or multiple aromatic rings fused together (e.g. naphthyl), or linked covalently, typically comprising 6 to 12 carbon atoms; wherein at least one ring is aromatic, preferably comprising 6 to 10 carbon atoms, wherein at least one ring is aromatic. The aromatic ring may optionally include one to two additional rings (either cycloalkyl, heterocyclyl or heteroaryl) fused thereto. Examples of suitable aryl include C₆₋₁₂aryl, preferably C₆₋₁₀aryl, more preferably C₆₋₈aryl. Non-limiting examples of aryl comprise phenyl, biphenylyl, biphenylenyl, or 1- or 2-naphthanelyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, and 1,4-dihydronaphthyl; 1-, 2-, 3-, 4- or 5-pyrenyl. A “substituted aryl” refers to an aryl group having one or more substituent(s) (for example 1, 2 or 3 substituent(s), or 1 to 2 substituent(s)), at any available point of attachment.

The term “aryloxy”, as a group or part of a group, refers to a group having the formula —OR^{g} wherein R^{g} is aryl as defined herein above.

The term “arylalkyl”, as a group or part of a group, means a alkyl as defined herein, wherein at least one hydrogen atom is replaced by at least one aryl as defined herein. Non-limiting examples of arylalkyl group include benzyl, phenethyl, dibenzylmethyl, methylphenylmethyl, 3-(2-naphthyl)-butyl, and the like.

The terms “heterocyclyl” or “heterocycloakyl” or “heterocyclo”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized, and wherein at least one carbon atom of heterocyclyl can be oxidized to form at least one C═O. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocyclic groups include aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, chromanyl (also known as 3,4-dihydrobenzo[b]pyranyl), isoindolinyl, 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “chromanyl” as used herein includes chroman-2-yl, chroman-3-yl, chroman-4-yl, chroman-5-yl, chroman-6-yl, chroman-7-yl and chroman-8-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl -3-yl and tetrahydrothiophenyl -4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl.

The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heterocyclyloxy”, as a group or part of a group, refers to a group having the formula —O—Rⁱ wherein Rⁱ is heterocyclyl as defined herein above.

The term “heterocyclylalkyl”, as a group or part of a group, means a alkyl as defined herein, wherein at least one hydrogen atom is replaced by at least one heterocyclyl as defined herein. The term “heteroaryl” as a group or part of a group, refers but is not limited to 5 to 12 carbon-atom aromatic rings or ring systems containing 1 or 2 rings which can be fused together or linked covalently, typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group consisting of pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

The term “heteroaryloxy”, as a group or part of a group, refers to a group having the formula —O—R^{k} wherein R^{k} is heteroaryl as defined herein above.

The term “heteroarylalkyl”, as a group or part of a group, means a alkyl as defined herein, wherein at least one hydrogen atom is replaced by at least one heteroaryl as defined herein.

The term “mono- or di-alkylamino”, as a group or part of a group, refers to a group of formula —N(R^{o})(R^{p}) wherein R^{o} and R^{p} are each independently selected from hydrogen, or alkyl, wherein at least one of R^{o} or R^{p} is alkyl. Thus, alkylamino include mono-alkyl amino group (e.g. mono-C₁₋₆alkylamino group such as methylamino and ethylamino), and di-alkylamino group (e.g. di-C₁₋₆alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “mono- or di-arylamino”, as a group or part of a group, refers to a group of formula —N(R^{q})(R^{r}) wherein R^{q} and R^{r} are each independently selected from hydrogen, or aryl, wherein at least one of R^{q} or R^{r} is aryl.

The term “mono- or di-arylalkylamino”, as a group or part of a group, refers to a group of formula —N(R^{q′})(R^{r′}) wherein R^{q′} and R^{r′} are each independently selected from hydrogen, or arylalkyl, wherein at least one of R^{q′} or R^{r′} is arylalkyl.

The term “mono- or di-cycloalkylamino”, as a group or part of a group, refers to a group of formula —N(R^{s})(R^{t}) wherein R^{s} and R^{t} are each independently selected from hydrogen, or cycloalkyl, wherein at least one of R^{s} or R^{t} is cycloalkyl.

The term “mono- or di-heteroarylamino”, as a group or part of a group, refers to a group of formula —N(R^{u})(R^{v}) wherein R^{u} and R^{v} are each independently selected from hydrogen, or heteroaryl, wherein at least one of R^{u} or R^{v} is heteroaryl as defined herein.

The term “mono- or di-heterocyclylamino”, as a group or part of a group, refers to a group of formula —N(R^{w})(R^{x}) wherein R^{w} and R^{x} are each independently selected from hydrogen, or heterocyclyl, wherein at least one of R^{w} or R^{x} is heterocyclyl as defined herein.

The term “alkyloxycarbonyl”, as a group or part of a group, refers to a group of formula —COO—R^{b}, wherein R^{b} is alkyl as defined herein.

The term “cycloakyloxycarbonyl”, as a group or part of a group, refers to a group of formula —COO—R^{b}, wherein R^{b} is cycloalkyl as defined herein.

The term “aryloxycarbonyl”, as a group or part of a group, refers to a group of formula —COO—R^{b}, wherein R^{b} is aryl as defined herein.

The term “heterocyclyloxycarbonyl”, as a group or part of a group, refers to a group of formula —COO—R^{b}, wherein R^{b} is heterocyclyl as defined herein.

The term “heteroaryloxycarbonyl”, as a group or part of a group, refers to a group of formula —COO—R^{b}, wherein R^{b} is heteroaryl as defined herein.

The term “alkylsulfinyl”, as a group or part of a group, refers to a group of formula —SO—R^{b}, wherein R^{b} is alkyl as defined herein.

The term “alkylsulfonyl”, as a group or part of a group, refers to a group of formula —S(O)₂—R^{b}, wherein R^{b} is alkyl as defined herein.

The term “cycloalkylsulfonyl”, as a group or part of a group, refers to a group of formula —S(O)₂—R^{b}, wherein R^{b} is cycloalkyl as defined herein.

The term “arylsulfonyl”, as a group or part of a group, refers to a group of formula —S(O)₂—R^{b}, wherein R^{b} is aryl as defined herein.

The term “heterocyclylsulfonyl”, as a group or part of a group, refers to a group of formula —S(O)₂—R^{b}, wherein R^{b} is heterocyclyl as defined herein.

The term “heteroarylsulfonyl”, as a group or part of a group, refers to a group of formula —S(O)₂—R^{b}, wherein R^{b} is heteroaryl as defined herein.

The term “mono- or di-alkylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is alkyl.

The term “mono- or di-cycloalkylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is cycloalkyl.

The term “mono- or di-arylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is aryl.

The term “mono- or di-heterocyclylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is heterocyclyl.

The term “mono- or di-heteroarylaminosulfonyl”, as a group or part of a group, refers to a group of formula —S(O)₂—NNR^{o}R^{p}, wherein R^{o}R^{p} are each independently selected from hydrogen, or alkyl, wherein at least one of R^{o} or R^{p} is heteroaryl.

The term “mono- or dialkylaminocarbonyl”, as a group or part of a group, refers to a group of formula —CONR^{o}R^{p} wherein R^{o}R^{p} are each independently selected from hydrogen, or alkyl, wherein at least one of R^{o} or R^{p} is alkyl.

The term “mono- or dicycloalkylaminocarbonyl”, as a group or part of a group, refers to a group of formula —CONR^{o}R^{p} wherein R^{o}R^{p} are each independently selected from hydrogen, or cycloalkyl, wherein at least one of R^{o} or R^{p} is cycloalkyl.

The term “alkylcarbonyl”, as a group or part of a group, refers to a group of formula —CO—R^{b}, wherein R^{b} is alkyl as defined herein.

The term “cycloalkylcarbonyl”, as a group or part of a group, refers to a group of formula —CO—R^{b}, wherein R^{b} is cycloalkyl as defined herein.

The term “arylcarbonyl”, as a group or part of a group, refers to a group of formula —CO—R^{b}, wherein R^{b} is aryl as defined herein.

The term “heterocyclylcarbonyl”, as a group or part of a group, refers to a group of formula —CO—R^{b}, wherein R^{b} is heterocyclyl as defined herein.

The term “heteroarylcarbonyl”, as a group or part of a group, refers to a group of formula —CO—R^{b}, wherein R^{b} is heteroaryl as defined herein.

The term “alkylcarbonylamino”, as a group or part of a group, refers to a group of formula —NR^{o}—CO—R^{b}, wherein R^{o} is selected from hydrogen, or alkyl and R^{b} is alkyl as defined herein.

The term “alkylsulfonylamino”, as a group or part of a group, refers to a group of formula —NR^{o}—S(O)₂—R^{b}, wherein R^{o} is selected from hydrogen, or alkyl and R^{b} is alkyl as defined herein.

Whenever used in the present invention the term “compounds of the invention” or a similar term is meant to include the compounds of general formula (I), as defined above, as well as (IA), (IIA), (IIB), (IIC), (IID) as detailed below and any subgroup thereof. This term also refers to the compounds as depicted in Table 1 and their derivatives, salts, solvates, hydrates, tautomeric forms, analogues, pro-drugs, esters and metabolites, as well as their quaternized nitrogen analogues.

As used herein and unless otherwise stated, the term “stereoisomer” refers to all possible different isomeric as well as conformational forms which the compounds of structural formula herein may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

The present invention includes all possible stereoisomers compounds of formula (I) and any subgroup thereof. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley- Interscience, 1994), incorporated by reference with regard to stereochemistry. A structural isomer is a type of isomer in which molecules with the same molecular formula have different bonding patterns and atomic organization. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

The term “prodrug” as used herein means the pharmacologically acceptable derivatives such as esters, amides and phosphates, such that the resulting in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Prodrugs of the compounds of the invention can be prepared by modifying functional groups present in said component in such a way that the modifications are cleaved, either in routine manipulation or in vivo, to the parent component. Typical examples of prodrugs are described for instance in WO 99/33795, WO 99/33815, WO 99/33793 and WO 99/33792 all incorporated herein by reference. Prodrugs are characterized by increased bio-availability and are readily metabolized into the active inhibitors in vivo. The term “prodrug”, as used herein, means any compound that will be modified to form a drug species, wherein the modification may take place either inside or outside of the body, and either before or after the pre-drug reaches the area of the body where administration of the drug is indicated.

Preferred statements (features) and embodiments of the compounds and processes of this invention are now set forth. Each statement and embodiment of the invention so defined may be combined with any other statement and/or embodiments unless clearly indicated to the contrary. In particular, any feature indicated as being preferred or advantageous may be combined with any other feature or features indicated as being preferred or advantageous.

A first aspect of the present invention provides a compound of formula (I) or a stereoisomer, or tautomer thereof,

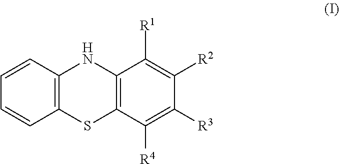

wherein,
R¹ is selected from the group consisting of hydrogen, amino, —NR⁶R⁷, halo, —C(O)NH(CR³R⁹)ₜR¹⁰, —C(O)R¹¹, —CR¹²R¹³NH(CR¹⁴R¹⁵)_{w}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z¹;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, cycloalkyl, aryl, heterocyclyl, and heteroaryl; wherein said cycloalkyl, aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z¹;
each R⁸, R⁹, R¹², R¹³, R¹⁴ and R¹⁵ is selected from the group consisting of hydrogen, halogen, hydroxyl, amino, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1b};
each R¹⁰, R¹¹ and R¹⁶ is selected from the group consisting of heterocyclyl, —NR⁶R⁷, alkyl, cycloalkyl, aryl, arylalkyl, heterocyclyl, heterocyclylalkyl, heteroaryl, and heteroarylalkyl; wherein said heterocyclyl, alkyl, cycloalkyl, aryl, arylalkyl, heterocyclylalkyl, heteroaryl or heteroarylalkyl can be unsubstituted or substituted with one or more Z^{1c};
each R¹⁷ is independently selected from the group consisting of alkyl, aryl, cycloalkyl, arylalkyl, heterocyclyl, heteroaryl;
each R¹⁸ and R¹⁹ is independently selected from the group consisting of hydrogen, alkyl, aryl, cycloalkyl, arylalkyl, heterocyclyl, heteroaryl;
each Z¹, Z², Z³, Z⁴, Z^{1a}, and Z^{1b} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano,
amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; each Z^{1c} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano, amino, —NR¹⁷R¹⁸, —C(O)₂R¹⁸, —C(O)NR¹⁸R¹⁹, —S(O)R¹⁸, —S(O)₂R¹⁸, —S(O)₂NR¹⁸R¹⁹, nitro;
wherein when R¹ is

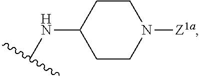

then R³ is not H, Cl or —C(O)R¹¹;
or a solvate, hydrate, pharmaceutically acceptable salt, or prodrug thereof;
with the proviso that said compound is not

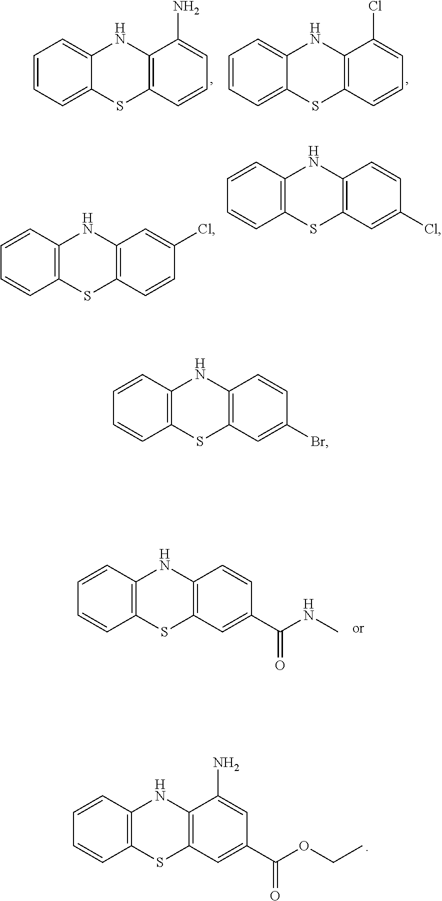

Another aspect of the present invention provides a compound of formula (I) or a stereoisomer, or tautomer thereof,

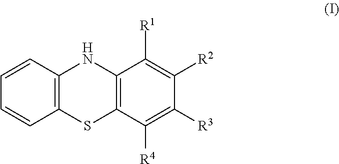

wherein,
R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z¹;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, cycloalkyl, aryl, heterocyclyl, and heteroaryl; wherein said cycloalkyl, aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1a};
each R⁸, R⁹, R¹², R¹³, R¹⁴ and R¹⁵ is selected from the group consisting of hydrogen, halogen, hydroxyl, amino, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1b};
each R¹⁰, R¹¹ and R¹⁶ is selected from the group consisting of heterocyclyl, —NR⁶R⁷, alkyl, cycloalkyl, aryl, arylalkyl, heterocyclyl, heterocyclylalkyl, heteroaryl, and heteroarylalkyl; wherein said heterocyclyl, alkyl, cycloalkyl, aryl, arylalkyl, heterocyclylalkyl, heteroaryl or heteroarylalkyl can be unsubstituted or substituted with one or more Z^{1c};
each R¹⁷ is independently selected from the group consisting of, alkyl, aryl, cycloalkyl, arylalkyl, heterocyclyl, heteroaryl;
each R¹⁸ and R¹⁹ is independently selected from the group consisting of hydrogen, alkyl, aryl, cycloalkyl, arylalkyl, heterocyclyl, heteroaryl;
each Z¹, Z², Z³, Z⁴, Z^{1a}, Z^{1b}, and Z^{1c} is independently selected from the group consisting of halo, haloalkyl, alkyl, haloalkyloxy, cycloalkyl, aryl, alkylaryl, heterocyclyl, heteroaryl, hydroxyl, —OR¹⁷, cyano,
amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; or a solvate, hydrate, pharmaceutically acceptable salt, or prodrug thereof;
with the proviso that said compound is not

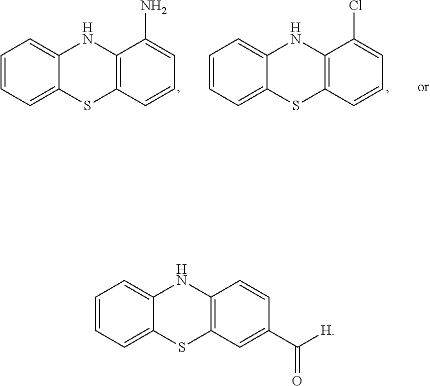

In some embodiments, the compound according to the invention has structural formula (IA),

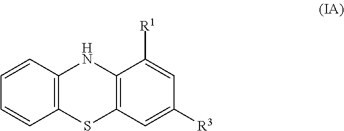

wherein R¹ and R³ have the same meaning as that defined herein.

In some embodiments, the compound according to the invention has structural formula (IIA), (III) (IIC) or (IID),

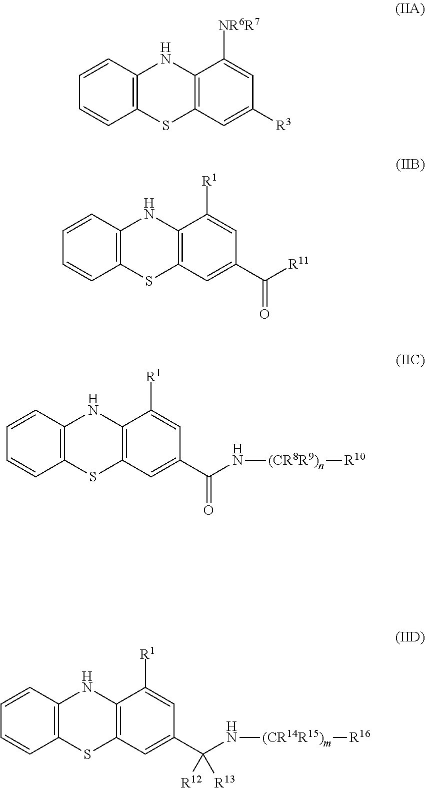

wherein n, m, R¹, R³, R⁶, R⁷, R⁸, R⁹, R¹⁰, R¹¹, R¹², R¹³, R¹⁴, R¹⁵, and R¹⁶ have the same meaning as that defined herein.

According to particular embodiments, the present invention provides compounds of formula (I), and any subgroup thereof such as (IA), (IIA), (IIB), (IIC), (IID), wherein,
R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z¹;
wherein t is an integer selected from 1, 2, or 3;
wherein w is an integer selected from 1, 2, or 3;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z²;
wherein n is an integer selected from 1, 2, or 3;
wherein m is an integer selected from 1, 2, or 3;
R³ is selected from the group consisting of hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z³;
wherein p is an integer selected from 1, 2, or 3;
wherein q is an integer selected from 1, 2, or 3;
R⁴ is selected from the group consisting of hydrogen, —NR⁶R⁷, amino, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z⁴;
wherein y is an integer selected from 1, 2, or 3;
wherein z is an integer selected from 1, 2, or 3
wherein at least one of R¹ to R⁴ is not hydrogen;
each R⁶ and R⁷ is independently selected from the group consisting of hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one, two or three Z^{1a};
each R⁸, R⁹, R¹², R¹³, R¹⁴ and R¹⁵ is selected from the group consisting of hydrogen, halogen, hydroxyl, amino, alkyl, cycloalkyl, and aryl;
each R¹⁰, R¹¹ and R¹⁶ is selected from the group consisting of heterocyclyl, —NR⁶R⁷, alkyl, arylalkyl, heterocyclylalkyl, and heteroarylalkyl; wherein said heterocyclyl, alkyl, arylalkyl, heterocyclylalkyl, or heteroarylalkyl can be unsubstituted or substituted with one, two or three Z^{1c}.

According to particular embodiments, the present invention provides compounds of formula (I), and any subgroup thereof such as (IA), (IIA), (IIB), (IIC), (IID), wherein,
R¹ is selected from the group consisting of hydrogen, amino, —NR⁶R⁷, halo, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl; can be unsubstituted or substituted with one or more Z¹;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, C₃₋₁₂cycloalkyl, C₆₋₁₂aryl, heterocyclyl, and heteroaryl; wherein said C₃₋₁₂cycloalkyl, C₆₋₁₂aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1a};
each R⁸, R⁹, R¹², R¹³, R¹⁴ and R¹⁵ is selected from the group consisting of hydrogen, halogen, hydroxyl, amino, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1b};
each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; wherein said heterocyclyl, C₁₋₆alkyl, C₃₋₁₂cycloalkyl, C₆₋₁₂aryl, C₆₋₁₂arylC₁₋₆alkyl, heterocyclylC₁₋₆ alkyl, heteroaryl or heteroarylC₁₋₆alkyl can be unsubstituted or substituted with one or more Z^{1c};
each R¹⁷ is independently selected from the group consisting of C₁₋₆alkyl, C₆₋₁₂aryl, C₃₋₁₂cycloalkyl, C₆₋₁₂arylC₁₋₆alkyl, heterocyclyl, heteroaryl;
each R¹⁸ and R¹⁹ is independently selected from the group consisting of hydrogen, C₁₋₆alkyl, C₆₋₁₂aryl, C₃₋₁₂cycloalkyl, C₆₋₁₂arylC₁₋₆alkyl, heteroaryl;
each Z¹, Z², Z³, Z⁴, Z^{1a}, and Z^{1b} is independently selected from the group consisting of halo, haloC₁₋₆alkyl, C₁₋₆alkyl, haloC₁₋₆alkyloxy, C₃₋₁₂cycloalkyl, C₆₋₁₂aryl, C₆₋₁₂arylC₁₋₆alkyl, heterocyclyl, heteroaryl, hydroxyl, —OR¹⁷, cyano, amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; each Z^{1c} is independently selected from the group consisting of halo, haloC₁₋₆alkyl C₁₋₆alkyl, haloC₁₋₆alkyloxy, C₃₋₁₂cycloalkyl, C₆₋₁₂aryl, C₆₋₁₂arylC₁₋₆alkyl, heterocyclyl, heteroaryl, hydroxyl, —OR¹⁷, cyano, amino, —NR¹⁷R¹⁸, —C(O)₂R¹⁸, —C(O)NR¹⁸R¹⁹, —S(O)R¹⁸, —S(O)₂R¹⁸, —S(O)₂NR¹⁸R¹⁹, nitro;
wherein when R¹ is

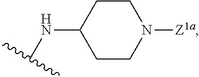

then R³ is not H, Cl or —C(O)R¹¹;
or a solvate, hydrate, pharmaceutically acceptable salt, or prodrug thereof.

According to particular embodiments, the present invention provides compounds of formula (I), and any subgroup thereof such as (IA), (IIA), (IIB), (IIC), (IID), wherein,
R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl; can be unsubstituted or substituted with one or more Z¹;
wherein t is an integer selected from 0, 1, 2, 3 or 4;
wherein w is an integer selected from 0, 1, 2, 3 or 4;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, and —SO₂NR⁶R⁷, C₃₋₁₂cycloalkyl, C₆₋₁₂aryl, heterocyclyl, and heteroaryl; wherein said C₃₋₁₂ cycloalkyl, C₆₋₁₂aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from the group consisting of hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z³;
wherein p is an integer selected from 0, 1, 2, 3 or 4;
wherein q is an integer selected from 0, 1, 2, 3 or 4;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1a};
each R⁸, R⁹, R¹², R¹³, R¹⁴ and R¹⁵ is selected from the group consisting of hydrogen, halogen, hydroxyl, amino, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1b};
each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; wherein said heterocyclyl, C₁₋₆alkyl, C₃₋₁₂cycloalkyl, C₆₋₁₂aryl, C₆₋₁₂arylC₁₋₆alkyl, heterocyclylC₁₋₆ alkyl, heteroaryl or heteroarylC₁₋₆alkyl can be unsubstituted or substituted with one or more Z^{1c};
each R¹⁷ is independently selected from the group consisting of C₁₋₆alkyl, C₆₋₁₂aryl, C₃₋₁₂cycloalkyl, C₆₋₁₂arylC₁₋₆alkyl, heterocyclyl, heteroaryl;
each R¹⁸ and R¹⁹ is independently selected from the group consisting of hydrogen, C₁₋₆alkyl, C₆₋₁₂aryl, C₃₋₁₂cycloalkyl, C₆₋₁₂arylC₁₋₆alkyl, heteroaryl;
each Z¹, Z², Z³, Z⁴, Z^{1a}, and Z^{1b} is independently selected from the group consisting of halo, halo C₁₋₆alkyl, C₁₋₆alkyl, halo C₁₋₆alkyl, C₃₋₁₂cycloalkyl, C₆₋₁₂aryl, C₆₋₁₂arylC₁₋₆alkyl, heterocyclyl, heteroaryl, hydroxyl, —OR¹⁷, cyano, amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; each Z^{1c} is independently selected from the group consisting of halo, halo C₁₋₆alkyl, C₁₋₆alkyl, halo C₁₋₆alkyl, C₃₋₁₂cycloalkyl, C₆₋₁₂aryl, C₆₋₁₂arylC₁₋₆alkyl, heterocyclyl, heteroaryl, hydroxyl, —OR¹⁷, cyano, amino, —NR¹⁷R¹⁸, —C(O)₂R¹⁸, —C(O)NR¹⁸R¹⁹, —S(O)R¹⁸, —S(O)₂R¹⁸, —S(O)₂NR¹⁸R¹⁹, nitro; or a solvate, hydrate, pharmaceutically acceptable salt, or prodrug thereof.

In some embodiments when R¹ is

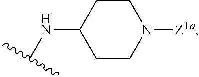

then R³ is not H, Cl or —C(O)R¹¹.

In some embodiments the compound according to the present invention is not is not

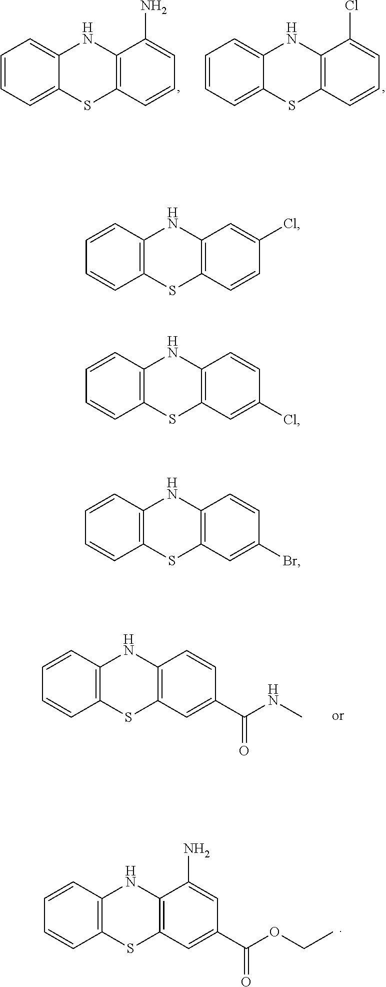

According to particular embodiments, the present invention provides compounds of formula (I), and any subgroup thereof such as (IA), (IIA), (IIB), (IIC), (IID), wherein,
R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, —SO₂NR⁶R⁷, C_{3- 12}cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z¹; preferably R¹ is hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R¹ is hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, C₆₋₁₂aryl, C₆₋₁₂arylC₁₋₆alkyl, heterocyclylC₁₋₆alkyl, and heteroarylC₁₋₆alkyl; preferably R¹ is hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, and —COOR¹¹; preferably R¹ is hydrogen, amino, —NR⁶R⁷, halo, and —COOR¹¹;
preferably said groups can be unsubstituted or substituted with one or more Z¹; preferably said groups can be unsubstituted or substituted with one, two or three Z¹;
wherein t is an integer selected from 0, 1, 2, 3 or 4; preferably t is 1, 2, or 3; preferably t is 1 or 2;
wherein w is an integer selected from 0, 1, 2, 3 or 4; preferably w is 1, 2, or 3; preferably w is 1 or 2;
R² is selected from the group consisting of hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, —SO₂NR⁶R⁷, C_{3- 12}cycloalkyl, C₆₋₁₂aryl, heterocyclyl, and heteroaryl; wherein said C₃₋₁₂cycloalkyl, C₆₋₁₂aryl, heterocyclyl, or heteroaryl can be unsubstituted or substituted with one or more Z²; preferably R² is hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, C₃₋₁₂cycloalkyl, C₆₋₁₂aryl, heterocyclyl, and heteroaryl; preferably R² is hydrogen, amino, —NR⁶R⁷, halo, NO₂, —C(O)NH(CR⁸R⁹)ₙR¹⁰, —C(O)R¹¹, —CR¹²R¹³NH(CR¹⁴R¹⁵)ₘR¹⁶, —COOR¹¹, C₆₋₁₂aryl, and heteroaryl; preferably R² is hydrogen, amino, —NR⁶R⁷, halo, NO₂, —C(O)NH(CR⁸R⁹)ₙR¹⁰, —C(O)R¹¹, and —CR¹²R¹³NH(CR¹⁴R¹⁵)ₘR¹⁶; preferably R² is hydrogen, amino, —NR⁶R⁷, halo, and —C(O)R¹¹; preferably R² is hydrogen;
preferably said groups can be unsubstituted or substituted with one or more Z²; preferably said groups can be unsubstituted or substituted with one, two or three Z²;
wherein n is an integer selected from 0, 1, 2, 3 or 4; preferably n is 1, 2, or 3; preferably n is 1 or 2;
wherein m is an integer selected from 0, 1, 2, 3 or 4; preferably n is 1, 2, or 3; preferably n is 1 or 2;
R³ is selected from the group consisting of hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, —SO₂NR⁶R⁷, C_{3- 12}cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z³; preferably R³ is hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R³ is hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, C₆₋₁₂arylC₁₋₆alkyl, heterocyclylC₁₋₆alkyl, and heteroarylC₁₋₆alkyl; preferably R³ is hydrogen, amino, —NR⁶R⁷, halo, NO₂, —C(O)NH(CR⁸R⁹)ₚR¹⁰, —C(O)R¹¹, and —CR¹²R¹³NH(CR¹⁴R¹⁵)_{q}R¹⁶; preferably R³ is hydrogen, —NR⁶R⁷, halo, —C(O)NH(CR⁸R⁹)ₚR¹⁰, —C(O)R¹¹, and —CR¹²R¹³NH(CR¹⁴R¹⁵)_{q}R¹⁶; preferably said groups can be unsubstituted or substituted with one or more Z³; preferably said groups can be unsubstituted or substituted with one, two or three Z³;
wherein p is an integer selected from 0, 1, 2, 3 or 4; preferably p is 1, 2, or 3; preferably p is 1 or 2;
wherein q is an integer selected from 0, 1, 2, 3 or 4; preferably q is 1, 2, or 3; preferably q is 1 or 2;
R⁴ is selected from the group consisting of hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, —SO₂NR⁶R⁷, C_{3- 12}cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z⁴; preferably R⁴ is hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R⁴ is hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, C₆₋₁₂arylC₁₋₆alkyl, heterocyclylC₁₋₆alkyl, and heteroarylC₁₋₆alkyl; preferably R⁴ is hydrogen, amino, —NR⁶R⁷, halo, NO₂, —C(O)NH(CR⁸R⁹)_{y}R¹⁰, —C(O)R¹¹, and —CR¹²R¹³NH(CR¹⁴R¹⁵)_{z}R¹⁶; preferably R⁴ is hydrogen, amino, —NR⁶R⁷, halo, and —C(O)R¹¹; preferably R⁴ is hydrogen;
preferably said groups can be unsubstituted or substituted with one or more Z⁴; preferably said groups can be unsubstituted or substituted with one, two or three Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from the group consisting of hydrogen, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1a}; preferably each R⁶ and R⁷ is independently selected from the group consisting of hydrogen, C₁₋₆alkyl, C₆₋₁₂arylC₁₋₆alkyl, heterocyclylC₁₋₆alkyl, and heteroarylC₁₋₆ alkyl; preferably each R⁶ and R⁷ is independently selected from the group consisting of hydrogen, C₁₋₆alkyl, and C₆₋₁₂arylC₁₋₆alkyl;
preferably said groups can be unsubstituted or substituted with one or more Z^{1a}; preferably said groups can be unsubstituted or substituted with one, two or three Z^{1a}each R⁸, R⁹, R¹², R¹³, R¹⁴ and R¹⁵ is selected from the group consisting of hydrogen, halogen, hydroxyl, amino, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1b}; preferably each R⁸, R⁹, R¹², R¹³, R¹⁴ and R¹⁵ is hydrogen, halogen, hydroxyl, amino, C₁₋₆alkyl, C₃₋₁₂cycloalkyl, C₆₋₁₂aryl, heterocyclyl, and heteroaryl; preferably each R⁸, R⁹, R¹², R¹³, R¹⁴ and R¹⁵ is hydrogen, halogen, amino, C₁₋₆alkyl, C₆₋₁₂aryl, heterocyclyl, and heteroaryl; preferably each R⁸, R⁹, R¹², R¹³, R¹⁴ and R¹⁵ is hydrogen, halogen, amino, and C₁₋₆alkyl;
preferably said groups can be unsubstituted or substituted with one or more Z^{1b}; preferably said groups can be unsubstituted or substituted with one, two or three Z^{1b};
each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; wherein said heterocyclyl, C₁₋₆alkyl, C₃₋₁₂cycloalkyl, C₆₋₁₂aryl, C₆₋₁₂arylC₁₋₆alkyl, heterocyclylC₁₋₆alkyl, heteroaryl or heteroarylC₁₋₆alkyl can be unsubstituted or substituted with one or more Z^{1c}; preferably each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; preferably each R¹⁰, R¹¹ and R¹⁶ is selected from the group consisting of heterocyclyl, —NR⁶R⁷, C₁₋₆alkyl, C₃₋₁₂cycloalkyl, and C₆₋₁₂aryl;
each R¹⁷ is independently selected from the group consisting of C₁₋₆alkyl, C₆₋₁₂aryl, C₃₋₁₂cycloalkyl, C₆₋₁₂arylC₁₋₆alkyl, heterocyclyl, heteroaryl; preferably each R¹⁷ is selected from C₁₋₆alkyl, C₆₋₁₂aryl, C₃₋₁₂cycloalkyl, heterocyclyl, and heteroaryl; preferably R¹⁷ is selected from C₁₋₆alkyl, C₆₋₁₂aryl, and C₃₋₁₂cycloalkyl;
each R¹⁸ and R¹⁹ is independently selected from the group consisting of hydrogen, C₁₋₆alkyl, C₆₋₁₂aryl, C₃₋₁₂cycloalkyl, C₆₋₁₂arylC₁₋₆alkyl, heteroaryl; preferably each R¹⁸ and R¹⁹ is selected from C₁₋₆alkyl, C₆₋₁₂aryl, C₃₋₁₂cycloalkyl, heterocyclyl, and heteroaryl; preferably R¹⁸ and R¹⁹ is selected from C₁₋₆alkyl, C₆₋₁₂aryl, and C₃₋₁₂cycloalkyl;
each Z¹, Z², Z³, Z⁴, Z^{1a}, Z^{1b}, and Z^{1c} is independently selected from the group consisting of halo, haloC₁₋₆alkyl, C₁₋₆alkyl, halo C₁₋₆alkyl, C₃₋₁₂cycloalkyl, C₆₋₁₂aryl, C₆₋₁₂arylC₁₋₆alkyl, heterocyclyl, heteroaryl, hydroxyl, —OR¹⁷, cyano, amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro; preferably each Z¹, Z², Z³, Z⁴, Z^{1a}, Z^{1b}, and Z^{1c} is selected from halo, C₁₋₆alkyl, haloC₁₋₄akyl, haloC₁₋₄akyloxy, C₃₋₁₂cycloalkyl, C₃₋₁₂cycloalkenyl, C₆₋₁₂aryl, heterocyclyl, heteroaryl, hydroxyl, —OR³, cyano, amino, —NR⁸R⁹, —C(O)₂R⁹, —C(O)NR⁹R¹⁰, —C(O)R⁸, —S(O)₂R⁹, —S(O)₂NR⁹R¹⁰; preferably each Z¹, Z², Z³, Z⁴, Z^{1a}, Z^{1b}, and Z^{1c} is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, amino, —NR⁸R⁹, —C(O)₂R⁹, —C(O)NR⁹R¹⁰, and —C(O)R⁸; preferably each Z¹, Z², Z³, Z⁴, Z^{1a}, Z^{1b}, and Z^{1c} is selected from halo, C₁₋₆alkyl, C₃₋₁₂cycloalkyl, C₃₋₁₂cycloalkenyl, C₆₋₁₂aryl, heterocyclyl, heteroaryl, hydroxyl, —OR⁸, cyano, —C(O)NR⁹R¹⁰, and —C(O)R⁸; preferably each Z¹, Z², Z³, Z⁴, Z^{1a}, Z^{1b}, and Z^{1c} is selected from halo, C₁₋₆alkyl, C₃₋₁₂cycloalkyl, C₃₋₁₂cycloalkenyl, C₆₋₁₂aryl, heterocyclyl, heteroaryl, and hydroxyl; preferably each Z¹, Z², Z³, Z⁴, Z^{1a}, Z^{1b}, and Z^{1c} is selected from halo, C₁₋₆alkyl, and hydroxyl;
or a solvate, hydrate, pharmaceutically acceptable salt, or prodrug thereof.

According to particular embodiments, the present invention provides compounds of formula (I), and any subgroup thereof such as (IA), (IIA), (IIB), (IIC), (IID),
wherein,
each Z¹ is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z² is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z³ is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄-akylaminosulfonyl, nitro;
each Z⁴ is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1a} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1b} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1c} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro.

According to particular embodiments, the present invention provides compounds of formula (I), and any subgroup thereof such as (IA), (IIA), (IIB), (IIC), (IID),
wherein,
each Z¹ is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z² is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z³ is independently selected from the group consisting of halo, C₁₋₆alkyl, haloC₁₋₄akyl, haloC₁₋₄akyloxy, C₃₋₁₂cycloalkyl, C₆₋₁₂aryl, C₁₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z⁴ is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1a} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1b} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1c} is independently selected from the group consisting of halo, C₁₋₆alkyl, haloC₁₋₄akyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, —S(O)H, C₁₋₄akylsulfinyl, —S(O)₂H, C₁₋₄akylsulfonyl, —SO₂NH₂, mono-C₁₋₄akylaminosulfonyl, nitro.

In some embodiments each Z¹, Z², Z³, Z⁴, Z^{1a}, Z^{1b}, and Z^{1c} is independently selected from the group consisting of halo, alkyl, haloalkyl, haloalkyloxy, cycloalkyl, aryl, alkylaryl, heterocyclyl, heteroaryl, hydroxyl, —OR¹⁸, cyano, amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹; preferably each Z¹, Z², Z³, Z⁴, Z^{1a}, Z^{1b}, and Z^{1c} is selected from halo, alkyl, haloalkyl, haloalkyloxy, cycloalkyl, aryl, alkylaryl, heterocyclyl, heteroaryl, hydroxyl, alkyloxy, cycloalkyloxy, aryloxy, heterocyclyloxy, heteroaryloxy, cyano, amino, mono-alkylamino, di-alkylamino, mono-cycloalkylamino, di-cycloalkylamino, mono-arylamino, di-arylamino, mono-heterocyclylamino, di-heterocyclylamino, mono-heteroarylamino, di-heteroarylamino, hydroxycarbonyl, alkyloxycarbonyl, cycloakyloxycarbonyl, aryloxycarbonyl, aminocarbonyl, mono-akylaminocarbonyl, di-akylaminocarbonyl, mono-cycloakylaminocarbonyl, alkylcarbonyl, cycloalkylcarbonyl, arylcarbonyl, —S(O)H, C₁₋₄akylsulfinyl, —S(O)₂H, C₁₋₄akylsulfonyl, —S(O)₂NH₂, mono-akylaminosulfonyl, di-akylaminosulfonyl, alkylcarbonylamino, alkylsulfonylamino, nitro; preferably each Z¹, Z², Z³, Z⁴, Z^{1a}, Z^{1b}, and Z^{1c} is selected from halo, alkyl, haloalkyl, haloalkyloxy, cycloalkyl, aryl, alkylaryl, heterocyclyl, heteroaryl, hydroxyl, alkyloxy, cycloalkyloxy, aryloxy, cyano, amino, mono-alkylamino, mono-cycloalkylamino, mono-arylamino, hydroxycarbonyl, alkyloxycarbonyl, cycloakyloxycarbonyl, aryloxycarbonyl, aminocarbonyl, mono-akylaminocarbonyl, mono-cycloakylaminocarbonyl, alkylcarbonyl, cycloalkylcarbonyl, —S(O)H, C₁₋₄akylsulfinyl, —S(O)₂H, C₁₋₄akylsulfonyl, —S(O)₂NH₂, mono-akylaminosulfonyl, alkylcarbonylamino, alkylsulfonylamino; preferably each Z¹, Z², Z³, Z⁴, Z^{1a}, Z^{1b}, and Z^{1c} is selected from halo, alkyl, haloalkyl, haloalkyloxy, cycloalkyl, aryl, heterocyclyl, heteroaryl, hydroxyl, alkyloxy, cycloalkyloxy, cyano, amino, mono-alkylamino, hydroxycarbonyl, alkyloxycarbonyl, aminocarbonyl, mono-akylaminocarbonyl, mono-cycloakylaminocarbonyl, alkylcarbonyl; preferably each Z¹, Z², Z³, Z⁴, Z^{1a}, Z^{1b}, and Z^{1c} is selected from halo, alkyl, haloalkyl, cycloalkyl, aryl, heterocyclyl, heteroaryl, hydroxyl, alkyloxy, cyano, amino, mono-alkylamino, aminocarbonyl, mono-cycloakylaminocarbonyl, alkylcarbonyl; preferably each Z¹, Z², Z³, Z⁴, Z^{1a}, Z^{1b}, and Z^{1c} is selected from halo, alkyl, haloalkyl, cycloalkyl, aryl, heterocyclyl, heteroaryl, hydroxyl, alkyloxy, cyano, amino; preferably each Z¹, Z², Z³, Z⁴, Z^{1a}, Z^{1b}, and Z^{1c} is selected from halo, alkyl, haloalkyl, cycloalkyl, aryl, hydroxyl, alkyloxy, cyano.

In some embodiments R¹ is selected from the group consisting of hydrogen, amino, —NR⁶R⁷, halo, NO₂, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; preferably R¹ is hydrogen, amino, mono-alkylamino, di-alkylamino, mono-cycloalkylamino, di-cycloalkylamino, mono-arylamino, di-arylamino, mono-arylalkylamino, di-arylalkylamino, mono-heterocyclylamino, di-heterocyclylamino, mono-heteroarylamino, di-heteroarylamino, halo, NO₂, —C(O)NH(CR⁸R⁹)ₜR¹⁰, heterocyclylcarbonyl, alkylcarbonyl, cycloalkylcarbonyl, arylcarbonyl, heteroarylcarbonyl, —CR¹²R¹³NH(CR¹⁴R¹⁵)_{w}R¹⁶, heterocyclyloxycarbonyl, alkyloxycarbonyl, cycloalkyloxyarbonyl, aryloxycarbonyl, heteroaryloxycarbonyl, alkylsulfonyl, cycloalkylsulfonyl, arylsulfonyl, heterocyclylsulfonyl, heteroarylsulfonyl, mono-alkylaminosulfonyl, di-alkylaminosulfonyl, mono-cycloalkylaminosulfonyl, di-cycloalkylaminosulfonyl, mono-arylaminosulfonyl, di-arylaminosulfonyl, mono-heterocyclylaminosulfonyl, di-heterocyclylaminosulfonyl, mono-heteroarylaminosulfonyl, di-heteroarylaminosulfonyl, cycloalkyl, aryl, arylalkyl, heterocyclyl, heterocyclylalkyl, heteroaryl and heteroarylalkyl; preferably R¹ is hydrogen, amino, mono-C₁₋₆alkylamino, di-C₁₋₆alkylamino, mono-C₃₋₁₂cycloalkylamino, di-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, di-heterocyclylamino, mono-heteroarylamino, di-heteroarylamino, halo, NO₂, —C(O)NH(CR⁸R⁹)ₜR¹⁰, heterocyclylcarbonyl, C₁₋₆alkylcarbonyl, C₃₋₁₂cycloalkylcarbonyl, C₆₋₁₂arylcarbonyl, heteroarylcarbonyl, —CR¹²R¹³NH(CR¹⁴R¹⁵)_{w}R¹⁶, heterocyclyloxycarbonyl, C₁₋₆alkyloxycarbonyl, C₃₋₁₂cycloalkyloxyarbonyl, C₆₋₁₂aryloxycarbonyl, heteroaryloxycarbonyl, C₁₋₆alkylsulfonyl, C₃₋₁₂cycloalkylsulfonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, di-C₃₋₁₂cycloalkylaminosulfonyl, mono-C₆₋₁₂arylaminosulfonyl, di-C₆₋₁₂arylaminosulfonyl, mono-heterocyclylaminosulfonyl, di-heterocyclylaminosulfonyl, mono-heteroarylaminosulfonyl, di-heteroarylaminosulfonyl, C₃₋₁₂cycloalkyl, C₃₋₁₂aryl, C₆₋₁₂arylC₁₋₆alkyl, heterocyclyl, heterocyclylC₁₋₆ alkyl, heteroaryl and heteroarylC₁₋₆alkyl; preferably R¹ is hydrogen, amino, mono-C₁₋₆alkylamino, di-C₁₋₆alkylamino, mono-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, mono-heteroarylamino, halo, NO₂, —C(O)NH(CR³R⁹)ₜR¹⁰, heterocyclylcarbonyl, C₃₋₁₂cycloalkylcarbonyl, C₆₋₁₂arylcarbonyl, heteroarylcarbonyl, —CR¹²R¹³NH(CR¹⁴R¹⁵)_{w}R¹⁶, heterocyclyloxycarbonyl, C₆₋₁₂aryloxycarbonyl, heteroaryloxycarbonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R¹ is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CR³R⁹)ₜR¹⁰, heterocyclylcarbonyl, C₆₋₁₂arylcarbonyl, heteroarylcarbonyl, —CR¹²R¹³NH(CR¹⁴R¹⁵)_{w}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R¹ is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CH₂)ₜR¹⁰, heterocyclylcarbonyl, C₆₋₁₂arylcarbonyl, heteroarylcarbonyl, —CH₂NH(CR¹⁴R¹⁵)_{w}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R¹ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CR³R⁹)ₜR¹⁰, heterocyclylcarbonyl, C₆₋₁₂arylcarbonyl, heteroarylcarbonyl, —CR¹²R¹³NH(CR¹⁴R¹⁵)_{w}R¹⁶, heterocyclyloxycarbonyl, and heteroaryloxycarbonyl; preferably R¹ is hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)ₜR¹⁰, heterocyclylcarbonyl, C₆₋₁₂arylcarbonyl, heteroarylcarbonyl, —CH₂NH(CR¹⁴R¹⁵)_{w}R¹⁶ heterocyclyloxycarbonyl, and heteroaryloxycarbonyl; preferably R¹ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, —C(O)NH(CH₂)ₜR¹⁰, heterocyclylcarbonyl, C₆₋₁₂ arylcarbonyl, heteroarylcarbonyl, heterocyclyloxycarbonyl, and heteroaryloxycarbonyl; preferably said groups can be unsubstituted or substituted with one or more Z¹; preferably said groups can be unsubstituted or substituted with one, two or three Z¹; preferably said groups can be unsubstituted or substituted with one, or two Z¹.

In some embodiments t is an integer selected from 0, 1, 2, 3 or 4; preferably t is 1, 2, or 3; preferably t is 1 or 2;

In some embodiments w is an integer selected from 0, 1, 2, 3 or 4; preferably w is 1, 2, or 3; preferably w is 1 or 2.

In some embodiments R² is selected from the group consisting of hydrogen, amino, —NR⁶R⁷, halo, NO₂, —C(O)NH(CR³R⁹)ₙR¹⁰, —C(O)R¹¹, —CR¹²R¹³NH(CR¹⁴R¹⁵)ₘR¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; preferably R² is hydrogen, amino, mono-alkylamino, di-alkylamino, mono-cycloalkylamino, di-cycloalkylamino, mono-arylamino, di-arylamino, mono-arylalkylamino, di-arylalkylamino, mono-heterocyclylamino, di-heterocyclylamino, mono-heteroarylamino, di-heteroarylamino, halo, NO₂, —C(O)NH(CR⁸R⁹)ₙR¹⁰, heterocyclylcarbonyl, alkylcarbonyl, cycloalkylcarbonyl, arylcarbonyl, heteroarylcarbonyl, —CR¹²R¹³NH(CR¹⁴R¹⁵)ₘR¹⁶, heterocyclyloxycarbonyl, alkyloxycarbonyl, cycloalkyloxyarbonyl, aryloxycarbonyl, heteroaryloxycarbonyl, alkylsulfonyl, cycloalkylsulfonyl, arylsulfonyl, heterocyclylsulfonyl, heteroarylsulfonyl, mono-alkylaminosulfonyl, di-alkylaminosulfonyl, mono-cycloalkylaminosulfonyl, di-cycloalkylaminosulfonyl, mono-arylaminosulfonyl, di-arylaminosulfonyl, mono-heterocyclylaminosulfonyl, di-heterocyclylaminosulfonyl, mono-heteroarylaminosulfonyl, di-heteroarylaminosulfonyl, cycloalkyl, aryl, arylalkyl, heterocyclyl, heterocyclylalkyl, heteroaryl and heteroarylalkyl; preferably R² is hydrogen, amino, mono-C₁₋₆alkylamino, di-C₁₋₆alkylamino, mono-C₃₋₁₂cycloalkylamino, di-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, di-heterocyclylamino, mono-heteroarylamino, di-heteroarylamino, halo, NO₂, —C(O)NH(CR⁸R⁹)ₙR¹⁰, heterocyclylcarbonyl, C₁₋₆alkylcarbonyl, C₃₋₁₂cycloalkylcarbonyl, C₆₋₁₂ arylcarbonyl, heteroarylcarbonyl, —CR¹²R¹³NH(CR¹⁴R¹⁵)ₘR¹⁶, heterocyclyloxycarbonyl, C₁₋₆alkyloxycarbonyl, C₃₋₁₂cycloalkyloxyarbonyl, C₆₋₁₂aryloxycarbonyl, heteroaryloxycarbonyl, C₁₋₆alkylsulfonyl, C₃₋₁₂cycloalkylsulfonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, di-C₃₋₁₂cycloalkylaminosulfonyl, mono-C₆₋₁₂arylaminosulfonyl, di-C₆₋₁₂arylaminosulfonyl, mono-heterocyclylaminosulfonyl, di-heterocyclylaminosulfonyl, mono-heteroarylaminosulfonyl, di-heteroarylaminosulfonyl, C₃₋₁₂cycloalkyl, C₆₋₁₂aryl, C₆₋₁₂arylC₁₋₆alkyl, heterocyclyl, heterocyclylC₁₋₆ alkyl, heteroaryl and heteroarylC₁₋₆alkyl; preferably R² is hydrogen, amino, mono-C₁₋₆alkylamino, di-C₁₋₆alkylamino, mono-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, mono-heteroarylamino, halo, NO₂, —C(O)NH(CR⁸R⁹)ₙR¹⁰, heterocyclylcarbonyl, C₃₋₁₂cycloalkylcarbonyl, C₆₋₁₂arylcarbonyl, heteroarylcarbonyl, —CR¹²R¹³NH(CR¹⁴R¹⁵)ₘR¹⁶, heterocyclyloxycarbonyl, C₆₋₁₂aryloxycarbonyl, heteroaryloxycarbonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R² is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CR³R⁹)ₙR¹⁰, heterocyclylcarbonyl, C₆₋₁₂arylcarbonyl, heteroarylcarbonyl, —CR¹²R¹³NH(CR¹⁴R¹⁵)ₘR¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R² is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CH₂)ₙR¹⁰, heterocyclylcarbonyl, C₆₋₁₂arylcarbonyl, heteroarylcarbonyl, —CH₂NH(CR¹⁴R¹⁵)ₘR¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R² is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CR³R⁹)ₙR¹⁰, heterocyclylcarbonyl, C₆₋₁₂arylcarbonyl, heteroarylcarbonyl, —CR¹²R¹³NH(CR¹⁴R¹⁵)ₘR¹⁶, heterocyclyloxycarbonyl, and heteroaryloxycarbonyl; preferably R² is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CH₂)ₙR¹⁰, heterocyclylcarbonyl, C₆₋₁₂arylcarbonyl, heteroarylcarbonyl, —CH₂NH(CR¹⁴R¹⁵)ₘR¹⁶, heterocyclyloxycarbonyl, and heteroaryloxycarbonyl; preferably R² is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, and iodo; preferably R² is hydrogen;
preferably said groups can be unsubstituted or substituted with one or more Z²; preferably said groups can be unsubstituted or substituted with one, two or three Z²; preferably said groups can be unsubstituted or substituted with one, or two Z².

In some embodiments n is an integer selected from 0, 1, 2, 3 or 4; preferably n is 1, 2, or 3; preferably n is 1 or 2; In some embodiments m is an integer selected from 0, 1, 2, 3 or 4; preferably m is 1, 2, or 3; preferably m is 1 or 2.

In some embodiments R³ is selected from the group consisting of hydrogen, amino, —NR⁶R⁷, halo, NO₂, —C(O)NH(CR³R⁹)ₚR¹⁰, —C(O)R¹¹, —CR¹²R¹³NH(CR¹⁴R¹⁵)_{q}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; preferably R³ is hydrogen, amino, mono-alkylamino, di-alkylamino, mono-cycloalkylamino, di-cycloalkylamino, mono-arylamino, di-arylamino, mono-arylalkylamino, di-arylalkylamino, mono-heterocyclylamino, di-heterocyclylamino, mono-heteroarylamino, di-heteroarylamino, halo, NO₂, —C(O)NH(CR⁸R⁹)ₚR¹⁰, heterocyclylcarbonyl, alkylcarbonyl, cycloalkylcarbonyl, arylcarbonyl, heteroarylcarbonyl, —CR¹²R¹³NH(CR¹⁴R¹⁵)_{q}R¹⁶, heterocyclyloxycarbonyl, alkyloxycarbonyl, cycloalkyloxyarbonyl, aryloxycarbonyl, heteroaryloxycarbonyl, alkylsulfonyl, cycloalkylsulfonyl, arylsulfonyl, heterocyclylsulfonyl, heteroarylsulfonyl, mono-alkylaminosulfonyl, di-alkylaminosulfonyl, mono-cycloalkylaminosulfonyl, di-cycloalkylaminosulfonyl, mono-arylaminosulfonyl, di-arylaminosulfonyl, mono-heterocyclylaminosulfonyl, di-heterocyclylaminosulfonyl, mono-heteroarylaminosulfonyl, di-heteroarylaminosulfonyl, cycloalkyl, aryl, arylalkyl, heterocyclyl, heterocyclylalkyl, heteroaryl and heteroarylalkyl; preferably R³ is hydrogen, amino, mono-C₁₋₆alkylamino, di-C₁₋₆alkylamino, mono-C₃₋₁₂cycloalkylamino, di-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, di-heterocyclylamino, mono-heteroarylamino, di-heteroarylamino, halo, NO₂, —C(O)NH(CR⁸R⁹)ₚR¹⁰, heterocyclylcarbonyl, C₁₋₆alkylcarbonyl, C₃₋₁₂cycloalkylcarbonyl, C₆₋₁₂arylcarbonyl, heteroarylcarbonyl, —CR¹²R¹³NH(CR¹⁴R¹⁵)_{q}R¹⁶, heterocyclyloxycarbonyl, C₁₋₆alkyloxycarbonyl, C₆₋₁₂cycloalkyloxyarbonyl, C₆₋₁₂aryloxycarbonyl, heteroaryloxycarbonyl, C₁₋₆alkylsulfonyl, C₃₋₁₂cycloalkylsulfonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, di-C₃₋₁₂cycloalkylaminosulfonyl, mono-C₆₋₁₂arylaminosulfonyl, di-C₆₋₁₂arylaminosulfonyl, mono-heterocyclylaminosulfonyl, di-heterocyclylaminosulfonyl, mono-heteroarylaminosulfonyl, di-heteroarylaminosulfonyl, C₃₋₁₂cycloalkyl, C₆₋₁₂aryl, C₆₋₁₂arylC₁₋₆alkyl, heterocyclyl, heterocyclylC₁₋₆ alkyl, heteroaryl and heteroarylC₁₋₆alkyl; preferably R³ is hydrogen, amino, mono-C₁₋₆alkylamino, di-C₁₋₆alkylamino, mono-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, mono-heteroarylamino, halo, NO₂, —C(O)NH(CR⁸R⁹)ₚR¹⁰, heterocyclylcarbonyl, C₃₋₁₂cycloalkylcarbonyl, C₆₋₁₂arylcarbonyl, heteroarylcarbonyl, —CR¹²R¹³NH(CR¹⁴R¹⁵)_{q}R¹⁶, heterocyclyloxycarbonyl, C₆₋₁₂aryloxycarbonyl, heteroaryloxycarbonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R³ is hydrogen, amino, mono-C₃₋₁₂cycloalkylamino, mono-C₆₋₁₂arylamino, di-C₆₋₁₂arylamino, mono-C₆₋₁₂arylC₁₋₆ alkylamino, mono-heterocyclylamino, mono-heteroarylamino, halo, NO₂, —C(O)NH(CR³R⁹)ₚR¹⁰, heterocyclylcarbonyl, C₆₋₁₂arylcarbonyl, heteroarylcarbonyl, —CR¹²R¹³NH(CR¹⁴R¹⁵)_{q}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R³ is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CH₂)ₚR¹⁰, heterocyclylcarbonyl, C₆₋₁₂arylcarbonyl, heteroarylcarbonyl, —CH₂NH(CR¹⁴R¹⁵)_{q}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R³ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CR³R⁹)ₚR¹⁰, heterocyclylcarbonyl, C₆₋₁₂arylcarbonyl, heteroarylcarbonyl, —CR¹²R¹³NH(CR¹⁴R¹⁵)_{q}R¹⁶, heterocyclyloxycarbonyl, and heteroaryloxycarbonyl; preferably R³ is hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)ₚR¹⁰, heterocyclylcarbonyl, C₆₋₁₂arylcarbonyl, heteroarylcarbonyl, —CH₂NH(CR¹⁴R¹⁵)_{q}R¹⁶, heterocyclyloxycarbonyl, and heteroaryloxycarbonyl; preferably R³ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, —C(O)NH(CH₂)ₚR¹⁰, heterocyclylcarbonyl, C₆₋₁₂ arylcarbonyl, heteroarylcarbonyl, —CH₂NH(CH₂)_{q}R¹⁶, heterocyclyloxycarbonyl, and heteroaryloxycarbonyl;
preferably said groups can be unsubstituted or substituted with one or more Z³; preferably said groups can be unsubstituted or substituted with one, two or three Z³; preferably said groups can be unsubstituted or substituted with one, or two Z³.

In some embodiments p is an integer selected from 0, 1, 2, 3 or 4; preferably p is 1, 2, or 3; preferably p is 1 or 2;

In some embodiments q is an integer selected from 0, 1, 2, 3 or 4; preferably q is 1, 2, or 3; preferably q is 1 or 2.

In some embodiments R⁴ is selected from the group consisting of hydrogen, amino, —NR⁶R⁷, halo, NO₂, —C(O)NH(CR³R⁹)_{y}R¹⁰, —C(O)R¹¹, —CR¹²R¹³NH(CR¹⁴R¹⁵)_{z}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; preferably R⁴ is hydrogen, amino, mono-alkylamino, di-alkylamino, mono-cycloalkylamino, di-cycloalkylamino, mono-arylamino, di-arylamino, mono-arylalkylamino, di-arylalkylamino, mono-heterocyclylamino, di-heterocyclylamino, mono-heteroarylamino, di-heteroarylamino, halo, NO₂, —C(O)NH(CR⁸R⁹)_{y}R¹⁰, heterocyclylcarbonyl, alkylcarbonyl, cycloalkylcarbonyl, arylcarbonyl, heteroarylcarbonyl, —CR¹²R¹³NH(CR¹⁴R¹⁵)_{z}R¹⁶, heterocyclyloxycarbonyl, alkyloxycarbonyl, cycloalkyloxyarbonyl, aryloxycarbonyl, heteroaryloxycarbonyl, alkylsulfonyl, cycloalkylsulfonyl, arylsulfonyl, heterocyclylsulfonyl, heteroarylsulfonyl, mono-alkylaminosulfonyl, di-alkylaminosulfonyl, mono-cycloalkylaminosulfonyl, di-cycloalkylaminosulfonyl, mono-arylaminosulfonyl, di-arylaminosulfonyl, mono-heterocyclylaminosulfonyl, di-heterocyclylaminosulfonyl, mono-heteroarylaminosulfonyl, di-heteroarylaminosulfonyl, cycloalkyl, aryl, arylalkyl, heterocyclyl, heterocyclylalkyl, heteroaryl and heteroarylalkyl; preferably R⁴ is hydrogen, amino, mono-C₁₋₆alkylamino, di-C₁₋₆alkylamino, mono-C₃₋₁₂cycloalkylamino, di-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, di-heterocyclylamino, mono-heteroarylamino, di-heteroarylamino, NO₂, —C(O)NH(CR⁸R⁹)_{y}R¹⁰, heterocyclylcarbonyl, C₁₋₆alkylcarbonyl, C₃₋₁₂cycloalkylcarbonyl, C₆₋₁₂ arylcarbonyl, heteroarylcarbonyl, —CR¹²R¹³NH(CR¹⁴R¹⁵)_{z}R¹⁶, heterocyclyloxycarbonyl, C₁₋₆alkyloxycarbonyl, C₃₋₁₂cycloalkyloxyarbonyl, C₆₋₁₂aryloxycarbonyl, heteroaryloxycarbonyl, C₁₋₆alkylsulfonyl, C₃₋₁₂cycloalkylsulfonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, di-C₃₋₁₂cycloalkylaminosulfonyl, mono-C₆₋₁₂arylaminosulfonyl, di-C₆₋₁₂arylaminosulfonyl, mono-heterocyclylaminosulfonyl, di-heterocyclylaminosulfonyl, mono-heteroarylaminosulfonyl, di-heteroarylaminosulfonyl, C₃₋₁₂cycloalkyl, C₆₋₁₂aryl, C₆₋₁₂arylC₁₋₆alkyl, heterocyclyl, heterocyclylC₁₋₆ alkyl, heteroaryl and heteroarylC₁₋₆alkyl; preferably R⁴ is hydrogen, amino, mono-C₁₋₆alkylamino, di-C₁₋₆alkylamino, mono-C₃₋₁₂cycloalkylamino, mono-C₆₋₁₂arylamino, di-C₆₋₁₂arylamino, mono-C₆₋₁₂arylC₁₋₆alkylamino, di-C₆₋₁₂arylC₁₋₆amino, mono-heterocyclylamino, mono-heteroarylamino, halo, NO₂, —C(O)NH(CR⁸R⁹)_{y}R¹⁰, heterocyclylcarbonyl, C₃₋₁₂cycloalkylcarbonyl, C₆₋₁₂arylcarbonyl, heteroarylcarbonyl, —CR¹²R¹³NH(CR¹⁴R¹⁵)_{z}R¹⁶, heterocyclyloxycarbonyl, C₆₋₁₂aryloxycarbonyl, heteroaryloxycarbonyl, C₆₋₁₂arylsulfonyl, heterocyclylsulfonyl, heteroarylsulfonyl, mono-C₁₋₆alkylaminosulfonyl, di-C₁₋₆alkylaminosulfonyl, mono-C₃₋₁₂cycloalkylaminosulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclyl, heterocyclylC₁₋₆alkyl, heteroaryl and heteroarylC₁₋₆alkyl; preferably R⁴ is hydrogen, amino, mono-C₃₋₁₂cycloalkylamino, mono-C₆₋₁₂arylamino, di-C₆₋₁₂arylamino, mono-C₆₋₁₂arylC₁₋₆alkylamino, mono-heterocyclylamino, mono-heteroarylamino, halo, NO₂, —C(O)NH(CR³R⁹)_{y}R¹⁰, heterocyclylcarbonyl, C₆₋₁₂arylcarbonyl, heteroarylcarbonyl, —CR¹²R¹³NH(CR¹⁴R¹⁵)_{z}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R⁴ is hydrogen, amino, mono-C₃₋₁₂cycloalkylamino, mono-C₆₋₁₂arylamino, di-C₆₋₁₂arylamino, mono-C₆₋₁₂arylC₁₋₆ alkylamino, mono-heterocyclylamino, mono-heteroarylamino, halo, NO₂, —C(O)NH(CH₂)_{y}R¹⁰, heterocyclylcarbonyl, C₆₋₁₂arylcarbonyl, heteroarylcarbonyl, —CH₂NH(CR¹⁴R¹⁵)_{z}R¹⁶, heterocyclyloxycarbonyl, heteroaryloxycarbonyl, heterocyclylsulfonyl, heteroarylsulfonyl, mono-C₆₋₁₂arylaminosulfonyl, mono-heterocyclylaminosulfonyl, mono-heteroarylaminosulfonyl, C₆₋₁₂aryl, C₆₋₁₂arylC₁₋₆alkyl, heterocyclylC₁₋₆alkyl, and heteroarylC₁₋₆alkyl; preferably R⁴ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CR³R⁹)_{y}R¹⁰, heterocyclylcarbonyl, C₆₋₁₂arylcarbonyl, heteroarylcarbonyl, —CR¹²R¹³NH(CR¹⁴R¹⁵)_{z}R¹⁶, heterocyclyloxycarbonyl, and heteroaryloxycarbonyl; preferably R⁴ is hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)_{y}R¹⁰, heterocyclylcarbonyl, C₆₋₁₂arylcarbonyl, heteroarylcarbonyl, —CH₂NH(CR¹⁴R¹⁵)_{z}R¹⁶, heterocyclyloxycarbonyl, and heteroaryloxycarbonyl; preferably R⁴ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, and iodo; preferably R⁴ is hydrogen; preferably said groups can be unsubstituted or substituted with one or more Z⁴; preferably said groups can be unsubstituted or substituted with one, two or three Z⁴; preferably said groups can be unsubstituted or substituted with one, or two Z⁴.

In some embodiments y is an integer selected from 0, 1, 2, 3 or 4; preferably y is 1, 2, or 3; preferably y is 1 or 2; In some embodiments z is an integer selected from 0, 1, 2, 3 or 4; preferably z is 1, 2, or 3; preferably z is 1 or 2.

In some embodiments each R⁶ and R⁷ is independently selected from the group consisting of hydrogen, C₁₋₆alkyl, C₃₋₁₂cycloalkyl, C₆₋₁₂aryl, C₆₋₁₂arylC₁₋₆alkyl, heterocyclyl, heterocyclylC₁₋₆ alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1a}; preferably each R⁶ and R⁷ is independently selected from the group consisting of hydrogen, C₁₋₆alkyl, C₆₋₁₂arylC₁₋₆alkyl, heterocyclylC₁₋₆alkyl, and heteroarylC₁₋₆alkyl; preferably each R⁶ and R⁷ is independently selected from the group consisting of hydrogen, C₁₋₆alkyl, and C₆₋₁₂arylC₁₋₆alkyl; preferably said groups can be unsubstituted or substituted with one or more Z^{1a}; preferably said groups can be unsubstituted or substituted with one, two or three Z^{1a}.

In some embodiments each R⁸, R⁹, R¹², R¹³, R¹⁴ and R¹⁵ is selected from the group consisting of hydrogen, halogen, hydroxyl, amino, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1b}; preferably each R⁸, R⁹, R¹², R¹³, R¹⁴ and R¹⁵ is hydrogen, halogen, hydroxyl, amino, C₁₋₆alkyl, C₃₋₁₂cycloalkyl, C₆₋₁₂aryl, heterocyclyl, and heteroaryl; preferably each R⁸, R⁹, R¹², R¹³, R¹⁴ and R¹⁵ is hydrogen, halogen, amino, C₁₋₆alkyl, C₆₋₁₂aryl, heterocyclyl, and heteroaryl; preferably said groups can be unsubstituted or substituted with one or more Z^{1b}; preferably said groups can be unsubstituted or substituted with one, two or three Z^{1b}.

In some embodiments each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; wherein said heterocyclyl, C₁₋₆alkyl, C₃₋₁₂cycloalkyl, C₆₋₁₂aryl, C₆₋₁₂arylC₁₋₆alkyl, heterocyclylC₁₋₆alkyl, heteroaryl or heteroarylC₁₋₆alkyl can be unsubstituted or substituted with one or more Z^{1c}; preferably each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl.

In some embodiments each R¹⁷ is independently selected from the group consisting of C₁₋₆alkyl, C₆₋₁₂aryl, C₃₋₁₂cycloalkyl, C₆₋₁₂arylC₁₋₆alkyl, heterocyclyl, heteroaryl; preferably each R¹⁷ is selected from C₁₋₆alkyl, C₆₋₁₂aryl, C₃₋₁₂cycloalkyl, heterocyclyl, and heteroaryl; preferably R¹⁷ is selected from C₁₋₆alkyl, C₆₋₁₂aryl, and C₃₋₁₂cycloalkyl.

In some embodiments each R¹⁸ and R¹⁹ is independently selected from the group consisting of hydrogen, C₁₋₆alkyl, C₆₋₁₂aryl, C₃₋₁₂cycloalkyl, C₆₋₁₂arylC₁₋₆alkyl, heteroaryl; preferably each R¹⁸ and R¹⁹ is selected from C₁₋₆alkyl, C₆₋₁₂aryl, C₃₋₁₂cycloalkyl, heterocyclyl, and heteroaryl; preferably R¹⁸ and R¹⁹ is selected from C₁₋₆alkyl, C₆₋₁₂aryl, and C₃₋₁₂cycloalkyl.

In some embodiments R² and R⁴ are hydrogen.

In some embodiments R¹ is selected from the group consisting of hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, NO₂, —C(O)NH(CH₂)ₜR¹⁰, heterocyclylcarbonyl, C₆₋₁₂arylcarbonyl, heteroarylcarbonyl, —CH₂NH(CR¹⁴R¹⁵)_{w}R¹⁶, heterocyclyloxycarbonyl, and heteroaryloxycarbonyl; preferably R¹ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, —C(O)NH(CH₂)ₜR¹⁰, heterocyclylcarbonyl, C₆₋₁₂arylcarbonyl, heteroarylcarbonyl, heterocyclyloxycarbonyl, and heteroaryloxycarbonyl;
preferably said groups can be unsubstituted or substituted with one or more Z¹; preferably said groups can be unsubstituted or substituted with one, two or three Z¹; preferably said groups can be unsubstituted or substituted with one, or two Z¹;
t is an integer selected from 1, 2, or 3; preferably t is 1 or 2;
w is an integer selected from 1, 2, or 3; preferably w is 1 or 2;
R² is selected from the group consisting of hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)ₙR¹⁰, heterocyclylcarbonyl, C₆₋₁₂arylcarbonyl, heteroarylcarbonyl, —CH₂NH(CR¹⁴R¹⁵)ₘR¹⁶, heterocyclyloxycarbonyl, and heteroaryloxycarbonyl; preferably R² is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, and iodo; preferably R² is hydrogen; preferably said groups can be unsubstituted or substituted with one or more Z²; preferably said groups can be unsubstituted or substituted with one, two or three Z²; preferably said groups can be unsubstituted or substituted with one, or two Z²;
n is an integer selected from 1, 2, or 3; preferably n is 1 or 2;
m is an integer selected from 1, 2, or 3; preferably m is 1 or 2;
R³ is selected from the group consisting of hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)ₚR¹⁰, heterocyclylcarbonyl, C₆₋₁₂arylcarbonyl, heteroarylcarbonyl, —CH₂NH(CR¹⁴R¹⁵)_{q}R¹⁶, heterocyclyloxycarbonyl, and heteroaryloxycarbonyl; preferably R³ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, iodo, —C(O)NH(CH₂)ₚR¹⁰, heterocyclylcarbonyl, C₆₋₁₂ arylcarbonyl, heteroarylcarbonyl, —CH₂NH(CH₂)_{q}R¹⁶, heterocyclyloxycarbonyl, and heteroaryloxycarbonyl;
preferably said groups can be unsubstituted or substituted with one or more Z³; preferably said groups can be unsubstituted or substituted with one, two or three Z³; preferably said groups can be unsubstituted or substituted with one, or two Z³.

p is an integer selected from 1, 2, or 3; preferably p is 1 or 2;
q is an integer selected from 1, 2, or 3; preferably q is 1 or 2;
R⁴ is selected from the group consisting of hydrogen, amino, mono-C₆₋₁₂arylamino, mono-C₆₋₁₂ arylC₁₋₆alkylamino, mono-heterocyclylamino, mono-heteroarylamino, fluoro, chloro, bromo, iodo, NO₂, —C(O)NH(CH₂)_{y}R¹⁰, heterocyclylcarbonyl, C₆₋₁₂arylcarbonyl, heteroarylcarbonyl, —CH₂NH(CR¹⁴R¹⁵)_{z}R¹⁶, heterocyclyloxycarbonyl, and heteroaryloxycarbonyl; preferably R⁴ is hydrogen, amino, mono-C₆₋₁₂arylamino, mono-C₆₋₁₂arylC₁₋₆alkylamino, mono-heterocyclylamino, mono-heteroarylamino, fluoro, chloro, bromo, and iodo; preferably R⁴ is hydrogen;
preferably said groups can be unsubstituted or substituted with one or more Z⁴; preferably said groups can be unsubstituted or substituted with one, two or three Z⁴; preferably said groups can be unsubstituted or substituted with one, or two Z⁴.

y is an integer selected from 1, 2, or 3; preferably y is 1 or 2;
z is an integer selected from 1, 2, or 3; preferably z is 1 or 2;
each R⁶ and R⁷ is independently selected from the group consisting of hydrogen, C₁₋₆alkyl, and C₆₋₁₂arylC₁₋₆alkyl; preferably said groups can be unsubstituted or substituted with one or more Z^{1a}; preferably said groups can be unsubstituted or substituted with one, two or three Z^{1a};
each R⁸, R⁹, R¹², R¹³, R¹⁴ and R¹⁵ is selected from the group consisting of hydrogen, halogen, hydroxyl, amino, C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₁₋₆alkyl, C₃₋₁₂cycloalkyl, C₆₋₁₂aryl, C₆₋₁₂arylC₁₋₆alkyl, heterocyclyl, heterocyclylC₁₋₆alkyl, heteroaryl or heteroarylC₁₋₆alkyl can be unsubstituted or substituted with one or more Z^{1b}; preferably each R⁸, R⁹, R¹², R¹³, R¹⁴ and R¹⁵ is hydrogen, halogen, hydroxyl, amino, C₁₋₆alkyl, C₃₋₁₂cycloalkyl, C₆₋₁₂aryl, heterocyclyl, and heteroaryl; preferably each R⁸, R⁹, R¹², R¹³, R¹⁴ and R¹⁵ is hydrogen, halogen, amino, C₁₋₆alkyl, C₆₋₁₂aryl, heterocyclyl, and heteroaryl; preferably said groups can be unsubstituted or substituted with one or more Z^{1b}; preferably said groups can be unsubstituted or substituted with one, two or three Z^{1b};
each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl; wherein said heterocyclyl, C₁₋₆alkyl, C₃₋₁₂cycloalkyl, C₆₋₁₂aryl, C₆₋₁₂arylC₁₋₆alkyl, heterocyclylC₁₋₆ alkyl, heteroaryl or heteroarylC₁₋₆alkyl can be unsubstituted or substituted with one or more Z^{1c}; preferably each R¹⁰, R¹¹ and R¹⁶ is selected from the group consisting of heterocyclyl, —NR⁶R⁷ C₁₋₆alkyl, C₃₋₁₂cycloalkyl, C₆₋₁₂aryl, C₆₋₁₂arylC₁₋₆alkyl, heterocyclylC₁₋₆alkyl, heteroaryl and heteroarylC₁₋₆alkyl;
each R¹⁷ is independently selected from the group consisting of C₁₋₆alkyl, C₆₋₁₂aryl, C₃₋₁₂cycloalkyl, C₆₋₁₂arylC₁₋₆alkyl, heterocyclyl, heteroaryl; preferably each R¹⁷ is selected from C₁₋₆alkyl, C₆₋₁₂aryl, C₃₋₁₂cycloalkyl, heterocyclyl, and heteroaryl; preferably R¹⁷ is selected from C₁₋₆alkyl, C₆₋₁₂aryl, and C₃₋₁₂cycloalkyl;
each R¹⁸ and R¹⁹ is independently selected from the group consisting of hydrogen, C₁₋₆alkyl, C₆₋₁₂aryl, C₃₋₁₂cycloalkyl, C₆₋₁₂arylC₁₋₆alkyl, heteroaryl; preferably each R¹⁸ and R¹⁹ is selected from C₁₋₆alkyl, C₆₋₁₂aryl, C₃₋₁₂cycloalkyl, heterocyclyl, and heteroaryl; preferably R¹⁸ and R¹⁹ is selected from C₁₋₆alkyl, C₆₋₁₂aryl, and C₃₋₁₂cycloalkyl;
each Z¹, Z², Z³, Z⁴, Z^{1a}, Z^{1b}, and Z^{1c} is selected from halo, alkyl, haloalkyl, cycloalkyl, aryl, heterocyclyl, heteroaryl, hydroxyl, alkyloxy, cyano, amino, mono-alkylamino, aminocarbonyl, mono-cycloakylaminocarbonyl, alkylcarbonyl; preferably each Z¹, Z², Z³, Z⁴, Z^{1a}, Z^{1b}, and Z^{1c} is selected from halo, alkyl, haloalkyl, cycloalkyl, aryl, heterocyclyl, heteroaryl, hydroxyl, alkyloxy, cyano, amino.

Particularly preferred compounds of the invention are those compounds listed in Table 1.

TABLE 1

| Compound | |
|---|---|
| code | Structure |
| CPD-001 | 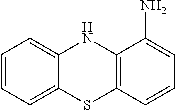 [SMILES: Nc1cccc2c1Nc1ccccc1S2] |
| CPD-002 | 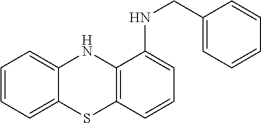 [SMILES: c1ccc(CNc2cccc3c2Nc2ccccc2S3)cc1] |
| CPD-003 | 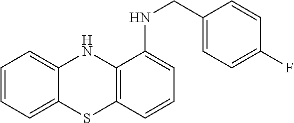 [SMILES: Fc1ccc(CNc2cccc3c2Nc2ccccc2S3)cc1] |
| CPD-004 | 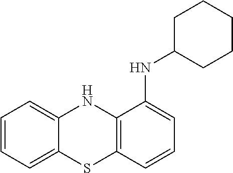 [SMILES: c1ccc2c(c1)Nc1c(NC3CCCCC3)cccc1S2] |
| CPD-005 | 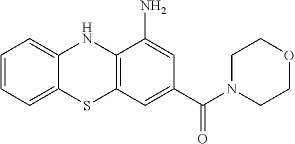 [SMILES: Nc1cc(C(=O)N2CCOCC2)cc2c1Nc1ccccc1S2] |
| CPD-006 | 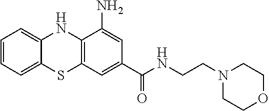 [SMILES: Nc1cc(C(=O)NCCN2CCOCC2)cc2c1Nc1ccccc1S2] |
| CPD-007 | 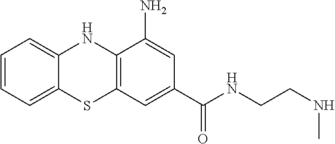 [SMILES: CNCCNC(=O)c1cc(N)c2c(c1)Sc1ccccc1N2] |
| CPD-008 | 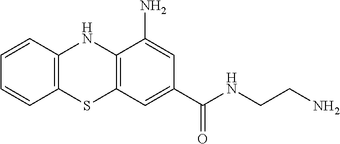 [SMILES: NCCNC(=O)c1cc(N)c2c(c1)Sc1ccccc1N2] |
| CPD-009 | 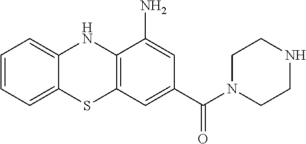 [SMILES: Nc1cc(C(=O)N2CCNCC2)cc2c1Nc1ccccc1S2] |
| CPD-010 | 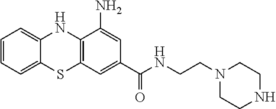 [SMILES: Nc1cc(C(=O)NCCN2CCNCC2)cc2c1Nc1ccccc1S2] |
| CPD-011 | 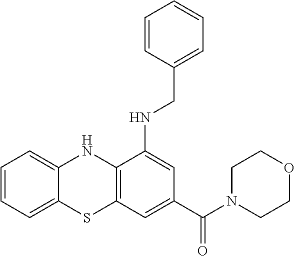 [SMILES: O=C(c1cc(NCc2ccccc2)c2c(c1)Sc1ccccc1N2)N1CCOCC1] |
| CPD-012 | 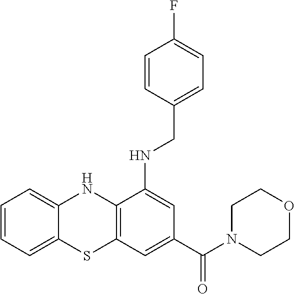 [SMILES: O=C(c1cc(NCc2ccc(F)cc2)c2c(c1)Sc1ccccc1N2)N1CCOCC1] |
| CPD-013 | 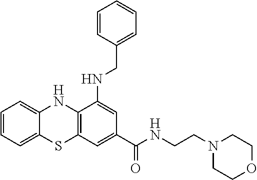 [SMILES: O=C(NCCN1CCOCC1)c1cc(NCc2ccccc2)c2c(c1)Sc1ccccc1N2] |
| CPD-014 | 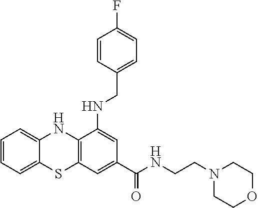 [SMILES: O=C(NCCN1CCOCC1)c1cc(NCc2ccc(F)cc2)c2c(c1)Sc1ccccc1N2] |
| CPD-015 | 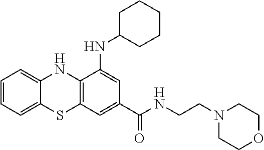 [SMILES: O=C(NCCN1CCOCC1)c1cc(NC2CCCCC2)c2c(c1)Sc1ccccc1N2] |
| CPD-016 | 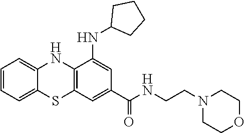 [SMILES: O=C(NCCN1CCOCC1)c1cc(NC2CCCC2)c2c(c1)Sc1ccccc1N2] |
| CPD-017 | 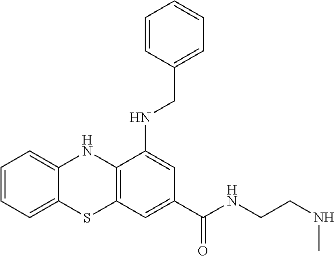 [SMILES: CNCCNC(=O)c1cc(NCc2ccccc2)c2c(c1)Sc1ccccc1N2] |
| CPD-018 | 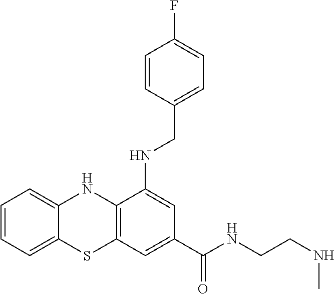 [SMILES: CNCCNC(=O)c1cc(NCc2ccc(F)cc2)c2c(c1)Sc1ccccc1N2] |
| CPD-019 | 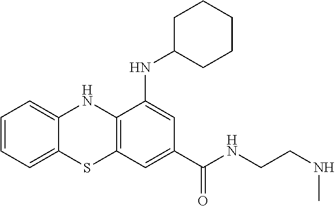 [SMILES: CNCCNC(=O)c1cc(NC2CCCCC2)c2c(c1)Sc1ccccc1N2] |
| CPD-020 | 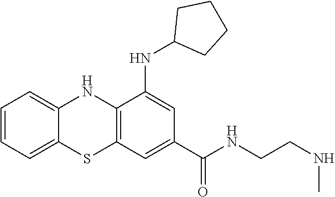 [SMILES: CNCCNC(=O)c1cc(NC2CCCC2)c2c(c1)Sc1ccccc1N2] |
| CPD-021 | 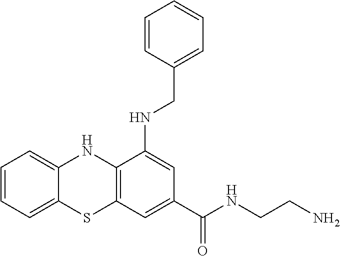 [SMILES: NCCNC(=O)c1cc(NCc2ccccc2)c2c(c1)Sc1ccccc1N2] |
| CPD-022 | 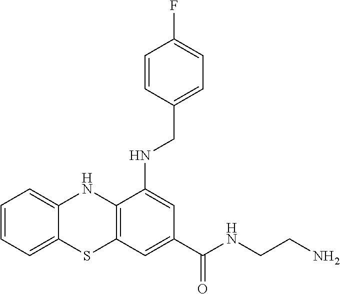 [SMILES: NCCNC(=O)c1cc(NCc2ccc(F)cc2)c2c(c1)Sc1ccccc1N2] |
| CPD-023 | 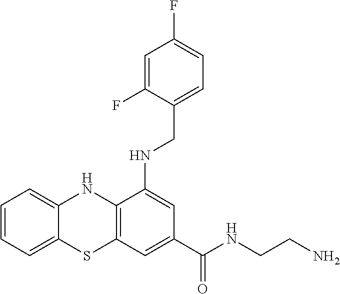 [SMILES: NCCNC(=O)c1cc(NCc2ccc(F)cc2F)c2c(c1)Sc1ccccc1N2] |
| CPD-024 | 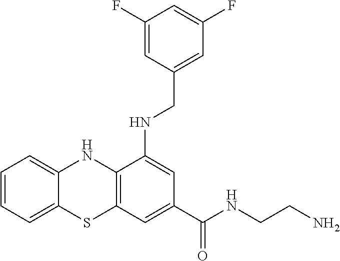 [SMILES: NCCNC(=O)c1cc(NCc2cc(F)cc(F)c2)c2c(c1)Sc1ccccc1N2] |
| CPD-025 | 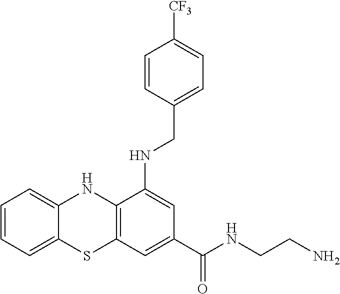 [SMILES: NCCNC(=O)c1cc(NCc2ccc(C(F)(F)F)cc2)c2c(c1)Sc1ccccc1N2] |
| CPD-026 | 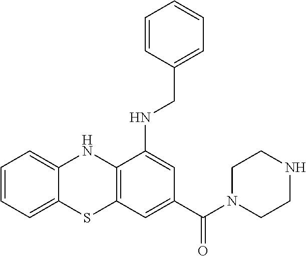 [SMILES: O=C(c1cc(NCc2ccccc2)c2c(c1)Sc1ccccc1N2)N1CCNCC1] |
| CPD-027 | 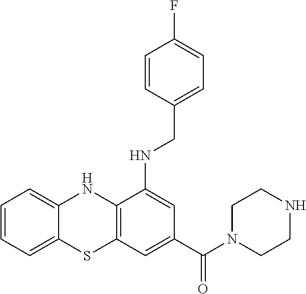 [SMILES: O=C(c1cc(NCc2ccc(F)cc2)c2c(c1)Sc1ccccc1N2)N1CCNCC1] |
| CPD-028 | 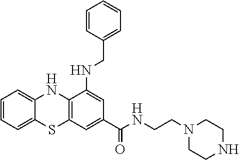 [SMILES: O=C(NCCN1CCNCC1)c1cc(NCc2ccccc2)c2c(c1)Sc1ccccc1N2] |
| CPD-029 | 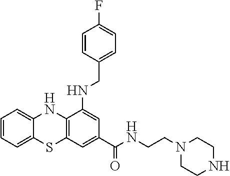 [SMILES: O=C(NCCN1CCNCC1)c1cc(NCc2ccc(F)cc2)c2c(c1)Sc1ccccc1N2] |
| CPD-030 | 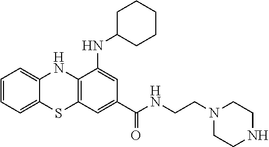 [SMILES: O=C(NCCN1CCNCC1)c1cc(NC2CCCCC2)c2c(c1)Sc1ccccc1N2] |
| CPD-031 | 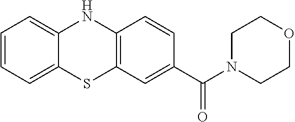 [SMILES: O=C(c1ccc2c(c1)Sc1ccccc1N2)N1CCOCC1] |
| CPD-032 | 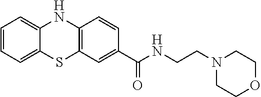 [SMILES: O=C(NCCN1CCOCC1)c1ccc2c(c1)Sc1ccccc1N2] |
| CPD-033 | 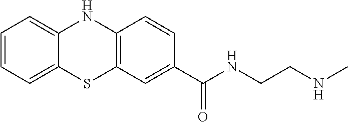 [SMILES: CNCCNC(=O)c1ccc2c(c1)Sc1ccccc1N2] |
| CPD-034 | 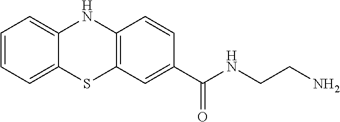 [SMILES: NCCNC(=O)c1ccc2c(c1)Sc1ccccc1N2] |
| CPD-035 | 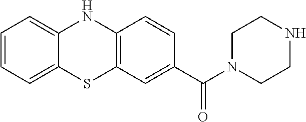 [SMILES: O=C(c1ccc2c(c1)Sc1ccccc1N2)N1CCNCC1] |
| CPD-036 | 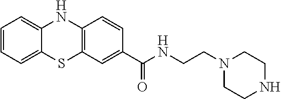 [SMILES: O=C(NCCN1CCNCC1)c1ccc2c(c1)Sc1ccccc1N2] |
| CPD-037 (comparative compound) | 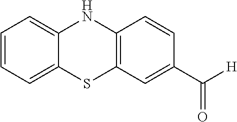 [SMILES: O=Cc1ccc2c(c1)Sc1ccccc1N2] |
| CPD-038 | 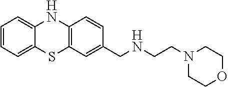 [SMILES: c1ccc2c(c1)Nc1ccc(CNCCN3CCOCC3)cc1S2] |
| CPD-039 (comparative compound) | 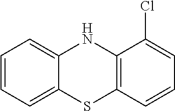 [SMILES: Clc1cccc2c1Nc1ccccc1S2] |

The compounds of the present invention have been found to be potent inhibitors of ferroptosis and/or oxytosis. Accordingly, the invention provides for the compounds of the invention for use in therapy. Their use is particularly advantageous in view of their properties making them particularly suited for use in vivo, i.e. demonstrating an improved effect in vivo.

More particularly, the compounds have been found to have moderate to high solubility and/or show high CNS MPO scores indicating that they are compounds with increased probability of success for use in the CNS.

A number of diseases are characterized by a dysregulation of ferroptosis and/or oxytosis, such as, but not limited to an excess in ferroptosis and/or oxytosis, causing cell-death. Accordingly, the present invention provides compounds of formula (I), and any subgroup thereof such as (I), (IA), (IIA), (IIB), (IIC), (IID), for use in the prevention or treatment of a disease associated with ferroptosis and/or oxytosis, more particularly an excess of ferroptosis and/or oxytosis leading to cell-death.

In a particular embodiment the invention provides the compounds of the invention or a pharmaceutical composition comprising one or more compounds of the invention for use in the treatment of a mammal suffering from excessive ferroptosis in one or more organs. The compounds of the invention are of use in a method of treatment or prevention of a disease characterized by a dysregulation of ferroptosis and/or oxytosis, such as, but not limited to an excess in ferroptosis and/or oxytosis, causing cell-death, which comprises administering one or more of the compounds of the invention to a patient suffering from said disease.

In some embodiments the disease associated with ferroptosis and/or oxytosis is selected from the group consisting of liver disease (NASH, NAFLD), chronic kidney disease, ocular surface diseases, wound healing, multiple organ dysfunction syndrome, neurological disease, acute renal failure, ischemia-reperfusion injury, sepsis, iron toxicity or iron poisoning, prevention of transplant rejection, and iron metabolism-related disease.

Non-limiting examples of disorders according to the present disclosure include epilepsy, kidney disease, stroke, myocardial infarction, type I diabetes, traumatic brain injury (TBI), periventricular leukomalacia (PVL), and neurodegenerative disease. Non-limiting examples of neurodegenerative diseases according to the present disclosure include Alzheimer's, Parkinson's, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, Progressive supranuclear palsy, Chronic Traumatic Encephalopathy (CTE), and Hereditary spastic paraparesis.

Non-limiting examples of liver disease include hemochromatosis, primary biliary cholangitis, non-alcoholic steatohepatitis or liver fibrosis.

Non-limiting examples of neurological disease include Alzheimer's Disease, Parkinson's Disease, Amyotrophic lateral sclerosis, Multiple Sclerosis, Huntington's Disease, Dementia with Lewy bodies, Friedreich's ataxia, multiple sclerosis, stroke, periventricular leukomalacia, intracerebral haemorrhage, frontotemporal dementia, neurodegeneration with brain iron accumulation, or traumatic brain injury.

Non-limiting examples of ischemia-reperfusion injury include myocardial ischemia-reperfusion injury, liver ischemia-reperfusion injury, lung ischemia-reperfusion injury, intestinal ischemia-reperfusion injury, renal ischemia-reperfusion injury or any surgical ischemia-reperfusion injury.

Non-limiting examples of iron metabolism-related disease include atherosclerosis or diabetes.

In a particular embodiment the invention provides the compounds of the invention or a pharmaceutical composition comprising one or more compounds of the invention for the treatment of a mammal suffering from excessive oxytosis in one or more organs.

In a particular embodiment the invention provides the compounds of the invention or a pharmaceutical composition comprising one or more compounds of the invention for the treatment of a mammal suffering from excessive ferroptosis and oxytosis in one or more organs.

In a particular embodiment the invention provides the compounds of the invention or a pharmaceutical composition comprising one or more compounds of the invention for the treatment of diseases caused by excitatory amino acids. A well-known example of an excitatory amino acid is glutamate and the condition is designated as glutamine excitotoxicity.

In a particular embodiment the invention provides the compounds of the invention or a pharmaceutical composition comprising one or more compounds of the invention for the treatment of diseases caused by increased levels of phospholipid peroxides.

In a particular embodiment the invention provides the compounds of the invention or a pharmaceutical composition comprising one or more compounds of the invention for the treatment of stroke, myocardial infarction, diabetes, sepsis, neurodegenerative diseases including Alzheimer's Disease, Parkinson's Disease, Amyotrophic lateral sclerosis, Huntington's Disease, Dementia with Lewy bodies, Friedreich's ataxia and multiple sclerosis. In a particular embodiment the invention provides the compounds of the invention or a pharmaceutical composition comprising one or more compounds of the invention for use in the prevention of transplant rejection. Thus the compounds can be used during the lung, intestine, kidney, liver or heart transplantation to prevent organ damage due to ischaemia-reperfusion injury.

In the present invention the potency of a compound inhibiting (or reducing) ferroptosis and/or oxytosis are determined in (bio)chemical antioxidant activity assays, in vitro assays. Typically FENIX assay is used to quantify radical trapping antioxidant activity in phospholipid bilayers to predict anti-ferroptotic potency of antioxidants in cells. Typically in vitro assays are used to measure the potency of a candidate compound. Examples of suitable in vitro assays are cellular assays. One non-limiting example of an assay involves the use of the IMR-32 neuroblastoma cell line. The latter is stimulated to enter into ferroptosis upon stimulation with 10 μM erastin, a documented ferroptosis inducer (see for example Dixon et al (2012) Cell 149, 1060-1072 and ferroptosis inhibitors are evaluated for the prevention of erastin induced ferroptosis. Another assay involves the use of ML162-induced ferroptosis in HT1080 human fibrosarcoma cells. Yet another assay is based the glutamate-induced cell death in the hippocampal cell line HT22 and ferroptosis/oxytosis inhibitors are evaluated for the prevention of cell death (see Henke N. et al (2013) Cell Death and Disease 4, e470). Still another assay is based on the sorafinib induced cell death (described to be iron dependent cell death) in hepatocellular carcinoma cells and ferroptosis inhibitors are evaluated for the prevention of cell death (see Louandre C. et al (2013) Int. J. Cancer 133, 1732). The calculated potency of a compound inhibiting ferroptosis and/or oxytosis is typically depicted as an IC50 value. Examples of suitable in vivo assays are typically pre-clinical disease models of for example mice for the diseases benefiting the application of ferroptosis and/or oxytosis inhibitors, as described herein.

One non-limiting example of an in vivo assay is based on inducing organ injury by iron overload in liver, kidney, lung or intestine-specific Gpx4 deficient mouse lines and ferroptosis inhibitors are evaluated based on the level of reduction in plasma injury biomarkers LDH, CK, AST and ALT, and body temperature (see Van Collie et al. (2022) Nat Comm 13: 1046).

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known therapeutic agents for the treatment of diseases mentioned herein, as well as with admixtures and combinations thereof. Particularly preferred combinations are necroptosis inhibitors (e.g. necrostatin-1) and ferroptosis inhibitors. Examples of these combinations are described in Linkerman et al2014.

The compounds of the invention may be in the form of salts, preferably pharmaceutically acceptable salts, as generally described below. Some preferred, but non-limiting examples of suitable pharmaceutically acceptable organic and/or inorganic acids are as hydrochloric acid, hydrobromic acid, sulfuric acid, nitric acid, acetic acid and citric acid, as well as other pharmaceutically acceptable acids known per se (for which reference is made to the prior art referred to below).

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of the compounds of formula (I) and any subgroup thereof include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Pharmaceutically acceptable salts of compounds of formula (I) may be prepared by one or more of these methods:
(i) by reacting the compound of formula (I) with the desired acid;
(ii) by reacting the compound of formula (I) with the desired base;
(iii) by removing an acid- or base-labile protecting group from a suitable precursor of the compound of formula (I) or by ring-opening a suitable cyclic precursor, for example, a lactone or lactam, using the desired acid; or
(iv) by converting one salt of the compound of formula (I) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

The compounds of the invention may also exist in unsolvated and solvated forms. The term ‘solvate’ is used herein to describe a molecular complex comprising the compound of the invention and one or more pharmaceutically acceptable solvent molecules, for example, ethanol.

The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Britain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na³⁰) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

All references to compounds of formula (I) or any subgroups thereof include references to salts, solvates, multi-component complexes and liquid crystals thereof and to solvates, multi-component complexes and liquid crystals of salts thereof.

The compounds of the invention include compounds of formula (I) or any subgroups thereof as hereinbefore defined, including all polymorphs and crystal habits thereof, prodrugs and isomers thereof (including optical, geometric and tautomeric isomers) as hereinafter defined and isotopically-labelled compounds of formula (I).

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention.

A further related aspect of the present invention provides a pharmaceutical composition comprising a compound of formula (I) or a stereoisomer, or tautomer thereof, and a pharmaceutically acceptable carrier.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

In preferred embodiments, the compound or the pharmaceutical composition as taught herein is administered parenterally. More preferably, the compound or the pharmaceutical composition as taught herein is administered intravenously, for example by infusion.

The dosage or amount of the agent as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the compound or the pharmaceutical composition as taught herein can be first administered at different dosing regimens. Typically, levels of the agent in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the agent as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilised. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

In particular embodiment, the compound as taught herein is the main or only active ingredient of the pharmaceutical composition.

### EXAMPLES

The following examples are provided for the purpose of illustrating the present invention and by no means should be interpreted to limit the scope of the present invention.

### 1. CHEMICAL SYNTHESIS OF REPRESENTATIVE COMPOUNDS OF THE INVENTION

Unless otherwise stated, laboratory reagent grade solvents were used. Reagents were obtained from various commercial sources and were used without any prior purification.

Characterisation of all compounds was done with ¹H and ¹³C NMR and mass spectrometry. NMR spectra were recorded with a 400 MHz Bruker Avance III Nanobay spectrometer with Ultrashield. All obtained spectra were analysed using MestReNova analytical chemistry software. Chemical shifts are displayed in ppm and coupling constants are shown in hertz (Hz). ES mass spectra were obtained from an Esquire 3000plus Ion Trap Mass Spectrometer from Bruker Daltonics.

The UPLC (ultra performance liquid chromatography), used to quantify the purity of the products, was an ACQUITY UPLC H-Class system with a TUV detector Waters coupled to an MS detector Waters Qda. Waters Acquity UPLC BEH C18 1.7 μm, 2.1 mm×50 mm column was used. The eluent was composed of two different solvents. Solvent C consisted of water with 0.1% formic acid, solvent D was acetonitrile. For most of the experiments, unless stated otherwise, the following general method was used. The column was first equilibrated for 0.15 min with a mixture of 95% solvent C and 5% solvent D. After that, solvent D was increased linearly to 100% over 1.75 min before being held constant for 0.25 min, followed by a mixture of 95% solvent C and 5% solvent D for 0.75 min (flow rate 0.7 ml/min). All mass spectra were recorded over a m/z range of 100-1000. The wavelength for UV detection was 254 nm.

Method II starts with equilibration of column for 0.15 min with a mixture of 95% solvent C and 5% solvent D. After that, solvent D was increased linearly to 100% over 2.50 min before being held constant for 0.75 min, followed by a mixture of 95% solvent C and 5% solvent D for 0.75 min (flow rate 0.7 ml/min).

Selected compounds of the invention were analyzed by high resolution mass spectrometry: 10 μL of each sample (concentration=10⁻⁵ M) was injected using the CapLC system (Waters, Manchester, UK) and electrosprayed using a standard electrospray source. Samples were injected with an interval of 5 min. Positive ion mode accurate mass spectra were acquired using a Q-TOF II instrument (Waters, Man-chester, UK). The MS was calibrated prior to use with a 0.2% H₃PO₄ solution. The spectra were lock mass corrected using the known mass of the nearest H₃PO₄ cluster.

During the chemical synthesis, flash purification was performed when necessary on a Biotage ISOLERA One flash system equipped with an internal variable dual wavelength diode array detector (200-400 nm). Reverse phase purifications were done using Buchi EcoFlex C18 cartridges, dry sample loading was done by self-packing sample cartridges using Celite® 545. Gradients used varied for each purification. Several synthesis procedures that were used in the preparation of intermediates and final products are summarized here as “General Procedures”.

All reactions were performed under argon unless otherwise stated. The final products have a purity above 95% unless otherwise stated.

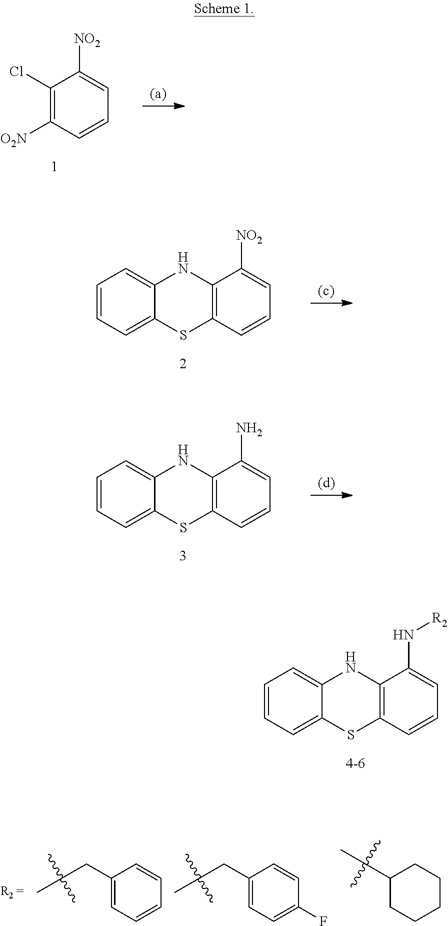

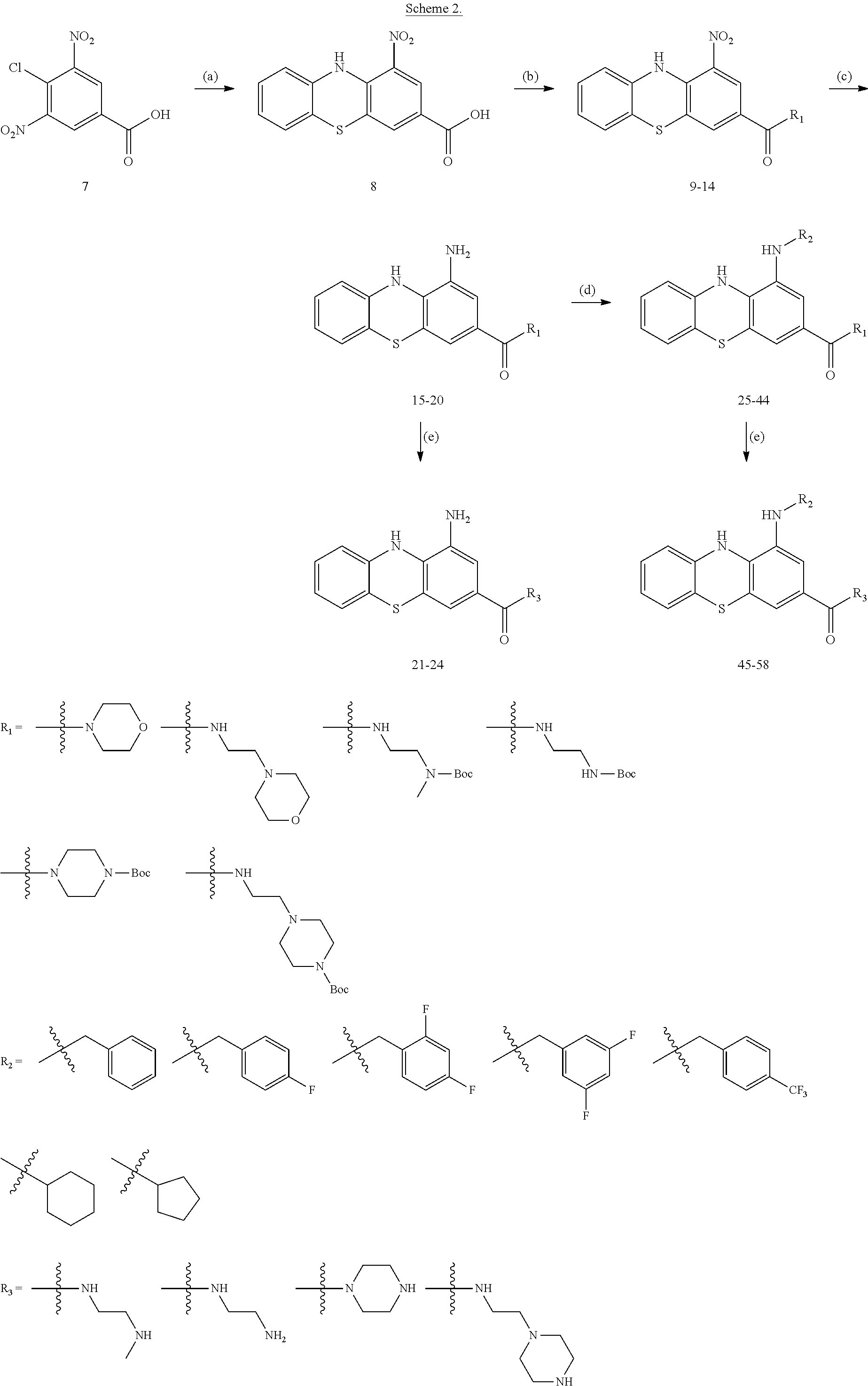

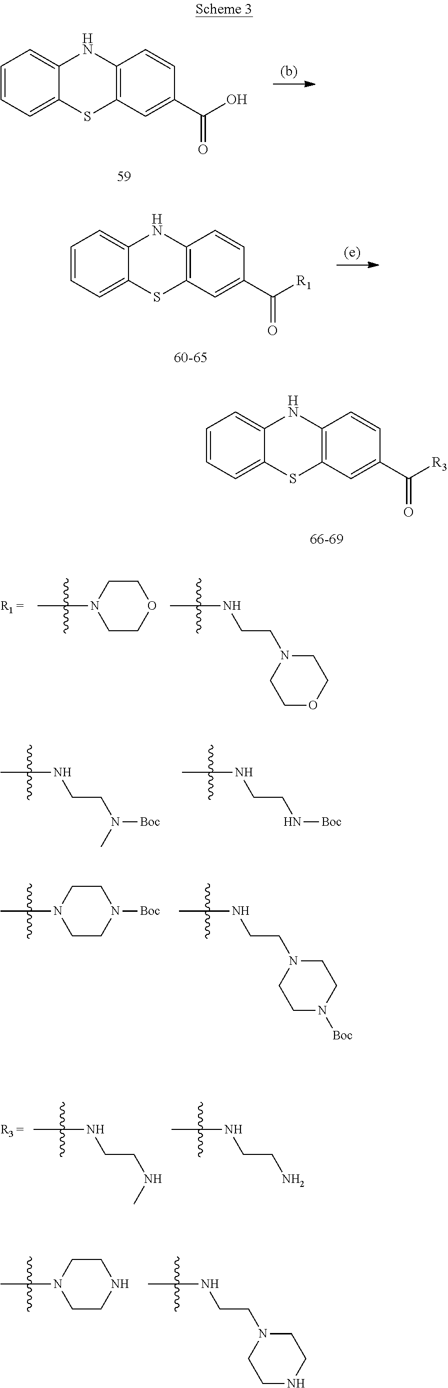

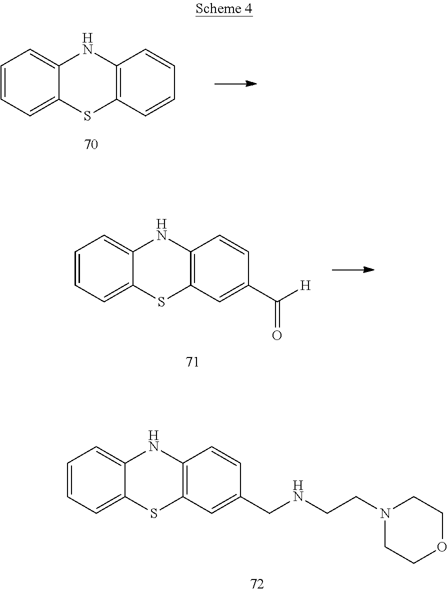 [SMILES: c1ccc2c(c1)Nc1ccccc1S2 ; O=Cc1ccc2c(c1)Sc1ccccc1N2 ; c1ccc2c(c1)Nc1ccc(CNCCN3CCOCC3)cc1S2]

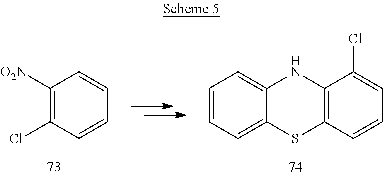 [SMILES: O=[N+]([O-])c1ccccc1Cl ; Clc1cccc2c1Nc1ccccc1S2]

### General Procedure A: Cross-Coupling Reaction for Phenothiazine Scaffold Synthesis (Schemes 1 or 2 Step (a)), Ref. Kumar et al. (2020) SN Applied Sciences, 2, 1241

To a stirred solution of 2-chloro-1,3-dinitrobenzene (1, 1.0 eq.) or 4-chloro-3,5-dinitrobenzoic acid (7, 1.0 eq.) in water, was added 2-aminothiophenol (1.0 eq.) at room temperature, followed by the addition of sodium hydroxide (1.1 eq. or more). The reaction mixture was heated to 85° C. or 60° C. respectively and stirred for 12-72 hours under reflux conditions. After completion of the reaction, the precipitate was filtered off, washed with water and dried under vacuum, providing compounds 2 or 8 respectively. The compounds were used in the next step without further purification.

### General Procedure B: Amide Coupling (Scheme 2 or 5 Step (b))

A stirred solution of compound 8 (1.0 eq.) in DMF or compound 59 (1.0 eq.) in THF was cooled to 0° C. in an ice bath, followed by the addition of HATU (1.4 eq.), DIPEA (3.0 eq.) and the corresponding amine (1.4 eq.). The reaction mixture was stirred for 12-72 hours at room temperature under argon. For reaction mixtures in DMF after completion, the solution was poured into ice water and stirred until precipitation occurred. The precipitate was then filtered off and washed with ice-cold water, yielding in intermediates 9-14. The obtained solids were used in the next step without further purification. For reaction mixtures in THF after completion, the solvent was evaporated and the crude compound was purified by reversed-phase flash chromatography (water/MeOH) to yield products 60-65.

### General Procedure C: Catalytic Hydrogenation (Schemes 1 or 2 Step (c))

Compounds 2 or 9-14 (1.0 eq.) were dissolved in methanol and the solution was purged with argon. Palladium hydroxide on carbon (20% Pd(OH)₂/C, 50% of water, 0.1 eq.) was added under inert atmosphere while continuously stirring. Next, the reaction mixture was put under hydrogen atmosphere and stirred for 12-72 hours at room temperature. After completion of the reaction, the solution was filtered through a patch of Celite and the filtrate was concentrated under reduced pressure. The crude compound was purified by reversed-phase flash chromatography (water/MeOH) to yield products 3 or 15-20.

### General Procedure D: Reductive Amination (Schemes 1 or 2 Step (d))

Compounds 3 or 15-20 (1.0 eq.) were dissolved in THF, followed by the addition of acetic acid (23.0 eq.) and the corresponding aldehyde or ketone (3.0 eq.). After 10 minutes, sodium cyanoborohydride (2.5 eq.) was added and the reaction mixture was stirred at room temperature for 12-16 hours. After completion, the reaction was quenched with water and the product was extracted twice into DCM. The combined organic layers were dried over sodium sulphate, filtered and loaded on Celite. The crude compounds were purified by reversed-phase flash chromatography (water/MeOH) to yield products 4-6 or 25-44.

### General Procedure E: Boc Deprotection (Scheme 2 or 5 Step (e))

Compounds 17-20 or 31-44 or 62-65 (1 eq.) were dissolved in dichloromethane followed by the addition of a 4 M solution of hydrochloric acid in dioxane (15 eq.) The reaction mixture was stirred at room temperature for 2 h. After reaction, diethyl ether was added to the reaction mixture in order to precipitate compounds 21-24 or 45-58 or 66-69. The obtained HCl salts were subsequently washed with a minimal amount of diethyl ether.

### Nitro-10H-phenothiazine (2)

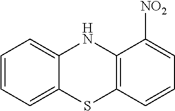 [SMILES: O=[N+]([O-])c1cccc2c1Nc1ccccc1S2]

General procedure A was followed using commercially available 2-chloro-1,3-dinitrobenzene (compound 1, 0.5 g, 2.469 mmol), 2-aminothiophenol (0.258 ml, 2.469 mmol) and sodium hydroxide (0.109 g, 2.72 mmol). The reaction mixture was stirred overnight to give 0.546 g (91%, crude yield) of compound 2.

¹H NMR (400 MHz, DMSO) δ 9.63 (s, 1H), 7.81 (d, J=8.6 Hz, 1H), 7.32 (d, J=7.5 Hz, 1H), 7.12-6.99 (m, 3H), 6.98-6.83 (m, 2H).

¹³C NMR (101 MHz, DMSO) δ 138.86, 138.09, 133.32, 132.39, 127.99, 126.19, 124.58, 124.22, 120.99, 120.90, 117.60, 117.10.

MS (ESI) m/z=244.0 [M]⁺.

### 10H-phenothiazin-1-amine (3, CPD-001)

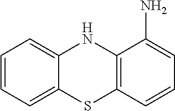 [SMILES: Nc1cccc2c1Nc1ccccc1S2]

General procedure C was followed using compound 2 (1.092 g, 4.47 mmol) and 20% Pd(OH)₂/C (50% of water, 0.238 g, 0.224 mmol). The reaction mixture was stirred for 24-72 hours and purified by reversed-phase column chromatography (10-70% MeOH in water), yielding 0.290 g of compound 3 (30%).

¹H NMR (400 MHz, DMSO) δ 7.52 (s, 1H), 6.99 (t, J=7.5 Hz, 1H), 6.92 (d, J=7.6 Hz, 1H), 6.84 (d, J=7.9 Hz, 1H), 6.75 (t, J=7.3 Hz, 1H), 6.57 (t, J=7.7 Hz, 1H), 6.45 (d, J=8.0 Hz, 1H), 6.23 (d, J=7.5 Hz, 1H), 5.06 (s, 2H).

¹³C NMR (101 MHz, DMSO) δ 143.24, 135.36, 128.15, 127.70, 126.55, 122.68, 122.11, 117.81, 117.13, 115.31, 115.14, 114.61.

MS (ESI) m/z=215.1 [M+H]⁺.

HRMS: calc 215.0637, found 215.0627 [M+H]⁺.

### N-benzyl-10H-phenothiazin-1-amine (4, CPD-002)

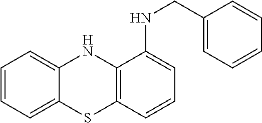 [SMILES: c1ccc(CNc2cccc3c2Nc2ccccc2S3)cc1]

General procedure D was followed using compound 3 (0.143 g, 0.665 mmol), acetic acid (0.875 ml, 15.29 mmol), benzaldehyde as corresponding aldehyde (0.204 ml, 1.995 mmol) and sodium cyanoborohydride (0.104 g, 1.662 mmol). The reaction mixture was stirred overnight and purified by reversed-phase column chromatography (10-75% MeOH in water). The reaction yielded 0.020 g of compound 4 (10%).

¹H NMR (400 MHz, DMSO) δ 7.77 (s, 1H), 7.41-7.29 (m, 4H), 7.29-7.20 (m, 1H), 7.01 (td, J=7.6, 1.5 Hz, 1H), 6.94 (dd, J=7.7, 1.4 Hz, 1H), 6.88 (dd, J=8.0, 1.3 Hz, 1H), 6.78 (td, J=7.4, 1.3 Hz, 1H), 6.60 (t, J=7.8 Hz, 1H), 6.35-6.23 (m, 2H), 5.89 (t, J=5.6 Hz, 1H), 4.34 (d, J=5.4 Hz, 2H).

¹³C NMR (101 MHz, DMSO) δ 142.79, 139.69, 135.12, 128.40, 128.30, 127.30, 126.84, 126.10, 122.28, 121.93, 117.61, 116.35, 115.06, 114.79, 110.19, 46.79.

Method II: MS (ESI) m/z=305.1 [M+H]⁺.

HRMS: calc 305.1107, found 305.1114 [M+H]⁺.

### N-(4-fluorobenzyl)-10H-phenothiazin-1-amine (5, CPD-003)

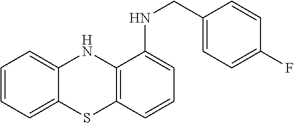 [SMILES: Fc1ccc(CNc2cccc3c2Nc2ccccc2S3)cc1]

General procedure D was followed using compound 3 (0.258 g, 1.203 mmol), acetic acid (1.582 ml, 27.64 mmol), 4-fluorobenzaldehyde as corresponding aldehyde (0.387 ml, 3.61 mmol) and sodium cyanoborohydride (0.189 g, 3.01 mmol). The reaction mixture was stirred overnight and purified by reversed-phase column chromatography (10-75% MeOH in water). The reaction yielded 0.029 g of compound 5 (7%).

¹H NMR (400 MHz, DMSO) δ 7.75 (s, 1H), 7.40 (dd, J=8.4, 5.5 Hz, 2H), 7.16 (t, J=8.7 Hz, 2H), 7.01 (t, J=7.4 Hz, 1H), 6.94 (d, J=7.6 Hz, 1H), 6.87 (d, J=7.8 Hz, 1H), 6.83-6.74 (m, 1H), 6.60 (t, J=7.8 Hz, 1H), 6.29 (dd, J=13.1, 7.8 Hz, 2H), 5.87 (t, J=5.6 Hz, 1H), 4.32 (d, J=5.4 Hz, 2H).

¹³C NMR (101 MHz, DMSO) δ 161.23 (d, J=242.0 Hz), 142.77, 135.81, 135.78, 134.96, 129.22 (d, J=8.1 Hz), 128.38, 127.34, 126.13, 122.31, 121.98, 117.61, 116.42, 115.14 (d, J=21.3 Hz), 115.01 (d, J=14.8 Hz), 110.24, 46.02.

MS (ESI) m/z=323.1 [M+H]⁺.

HRMS: calc 323.1013, found 323.1013 [M+H]⁺.

### N-cyclohexyl-10H-phenothiazin-1-amine (6, CPD-004)

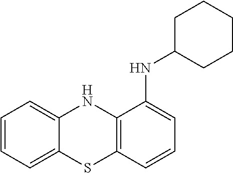 [SMILES: c1ccc2c(c1)Nc1c(NC3CCCCC3)cccc1S2]

General procedure D was followed using compound 3 (0.035 g, 0.163 mmol), acetic acid (0.215 ml, 3.76 mmol), cyclohexanone as corresponding ketone (0.051 ml, 0.490 mmol) and sodium cyanoborohydride (0.026 g, 0.408 mmol). The reaction mixture was stirred overnight and purified by reversed-phase column chromatography (10-75% MeOH in water). The reaction yielded 0.016 g of compound 6 (33%).

¹H NMR (400 MHz, DMSO) δ 7.69 (s, 1H), 7.04-6.97 (m, 1H), 6.95-6.84 (m, 2H), 6.77 (td, J=7.4, 1.3 Hz, 1H), 6.66 (t, J=7.8 Hz, 1H), 6.46-6.39 (m, 1H), 6.25 (dd, J=7.6, 1.2 Hz, 1H), 4.88 (d, J=7.2 Hz, 1H), 3.19 (dtd, J=10.3, 6.4, 3.3 Hz, 1H), 2.03-1.94 (m, 2H), 1.74 (dt, J=12.8, 3.5 Hz, 2H), 1.63 (d, J=12.5 Hz, 1H), 1.42-1.26 (m, 2H), 1.18 (qd, J=12.2, 3.2 Hz, 3H).

¹³C NMR (101 MHz, DMSO) δ 142.87, 134.42, 128.22, 127.22, 126.06, 122.40, 121.82, 117.73, 116.62, 115.01, 114.33, 110.47, 51.40, 32.87, 25.66, 24.83.

MS (ESI) m/z=297.1 [M+H]⁺.

### 1-Nitro-10H-phenothiazine-3-carboxylic acid (8)

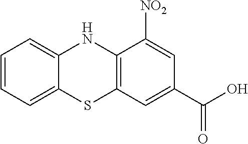 [SMILES: O=C(O)c1cc2c(c([N+](=O)[O-])c1)Nc1ccccc1S2]

General procedure A was followed using commercially available 4-chloro-3,5-dinitrobenzoic acid (compound 7, 0.5 g, 2.028 mmol), 2-aminothiophenol (0.212 ml, 2.028 mmol) and sodium hydroxide (0.089 g, 2.231 mmol). The reaction mixture was stirred overnight to give 0.456 g (78%, crude yield) of compound 8.

¹H NMR (400 MHz, DMSO) δ 9.88 (s, 1H), 8.27 (d, J=1.9 Hz, 1H), 7.66 (d, J=1.9 Hz, 1H), 7.10 (d, J=4.1 Hz, 2H), 7.05 (d, J=7.6 Hz, 1H), 6.99 (dq, J=8.0, 4.2 Hz, 1H).

¹³C NMR (101 MHz, DMSO) δ 165.42, 142.03, 137.29, 132.90, 131.69, 128.60, 126.69, 126.47, 125.85, 123.35, 121.61, 118.64, 117.36.

MS (ESI) m/z=287.0 [M−H]⁻.

### Morpholino(1-nitro-10H-phenothiazin-3-yl)methanone (9)

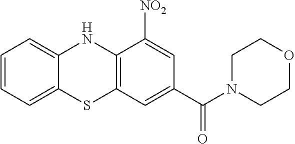 [SMILES: O=C(c1cc2c(c([N+](=O)[O-])c1)Nc1ccccc1S2)N1CCOCC1]

General procedure B was followed using compound 8 (0.55 g, 1.694 mmol), morpholine as corresponding amine (0.205 ml, 2.371 mmol), HATU (0.902 g, 2.371 mmol) and DIPEA (0.885 ml, 5.08 mmol). The reaction mixture was stirred for 72 hours to give 0.9026 g (>99%, crude yield) of compound 9.

¹H NMR (400 MHz, DMSO) δ 9.76 (s, 1H), 7.83 (d, J=1.9 Hz, 1H), 7.39 (d, J=1.9 Hz, 1H), 7.13-7.03 (m, 3H), 7.02-6.94 (m, 1H), 3.73-3.40 (m, 8H).

¹³C NMR (101 MHz, DMSO) δ 166.26, 139.63, 137.50, 132.54, 130.62, 128.20, 127.70, 126.32, 125.08, 123.61, 121.40, 117.96, 116.91, 66.02.

MS (ESI) m/z=358.1 [M+H]⁺.

### N-(2-morpholinoethyl)-1-nitro-10H-phenothiazine-3-carboxamide (10)

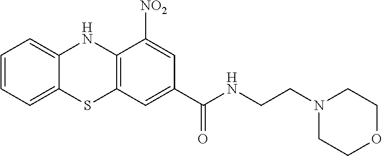 [SMILES: O=C(NCCN1CCOCC1)c1cc2c(c([N+](=O)[O-])c1)Nc1ccccc1S2]

General procedure B was followed using compound 8 (0.456 g, 1.404 mmol), 2-morpholinoethan-1-amine as corresponding amine (0.256 g, 1.966 mmol), HATU (0.748 g, 1.966 mmol) and DIPEA (0.734 ml, 4.21 mmol). The reaction mixture was stirred for 72 hours to provide 0.500 g (89%, crude yield) of compound 10.

¹H NMR (400 MHz, DMSO) δ 9.79 (s, 1H), 8.55 (t, J=5.7 Hz, 1H), 8.32 (d, J=2.0 Hz, 1H), 7.72 (d, J=2.0 Hz, 1H), 7.12-7.02 (m, 3H), 6.98 (dq, J=8.0, 4.4 Hz, 1H), 3.55 (t, J=4.6 Hz, 4H), 3.38-3.35 (m, 2H), 2.41 (dd, J=14.7, 7.7 Hz, 6H).

¹³C NMR (101 MHz, DMSO) δ 163.12, 140.46, 137.22, 132.59, 129.97, 128.13, 126.37, 126.25, 125.14, 123.73, 120.83, 118.04, 116.81, 66.19, 57.30, 53.30, 36.64.

MS (ESI) m/z=401.2 [M+H]⁺.

### tert-Butylmethyl(2-(1-nitro-10H-phenothiazine-3-carboxamido)ethyl)carbamate (11)

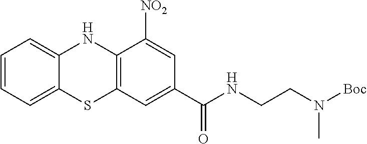 [SMILES: CN(CCNC(=O)c1cc2c(c([N+](=O)[O-])c1)Nc1ccccc1S2)C(=O)OC(C)(C)C]

General procedure B was followed using compound 8 (1.337 g, 4.12 mmol), tert-butyl 2-(methylamino)ethylcarbamate as corresponding amine (0.736 ml, 4.12 mmol), HATU (2.191 g, 5.76 mmol) and DIPEA (2.151 ml, 12.35 mmol). The reaction mixture was stirred for 72 hours to provide 1.621 g (89%, crude yield) of compound 11.

¹H NMR (400 MHz, DMSO) b 9.78 (s, 1H), 8.72-8.57 (m, 11H), 8.41-8.22 (m, 1H), 7.80-7.61 (m, 1H), 7.18-6.91 (m, 4H), 3.32 (s, 3H), 3.00-2.63 (m, 4H), 1.48-1.21 (m, 9H).

¹³C NMR (101 MHz, DMSO) δ 163.65, 162.77, 155.56, 155.22, 150.20, 140.85, 137.56, 135.87, 132.90, 131.59, 130.38, 128.55, 126.65, 125.57, 124.16, 121.26, 118.45, 117.22, 116.92, 116.51, 115.24, 78.94, 78.73, 48.11, 47.64, 37.95, 37.73, 36.25, 34.79, 34.41, 31.23, 28.45.

MS (ESI) m/z=345.1 [M−Boc+H]³⁰.

### tert-Butyl (2-(1-nitro-10H-phenothiazine-3-carboxamido)ethyl)carbamate (12)

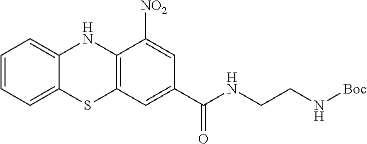 [SMILES: CC(C)(C)OC(=O)NCCNC(=O)c1cc2c(c([N+](=O)[O-])c1)Nc1ccccc1S2]

General procedure B was followed using compound 8 (1.4 g, 4.31 mmol), N-Boc-ethylenediamine as corresponding amine (0.956 ml, 6.04 mmol), HATU (2.295 g, 6.04 mmol) and DIPEA (2.253 ml, 12.93 mmol). The reaction mixture was stirred overnight to provide 1.73 g (93%, crude yield) of compound 12.

¹H NMR (400 MHz, DMSO) δ 9.75 (s, 1H), 8.56-8.51 (m, 1H), 8.27 (d, J=2.0 Hz, 1H), 7.70-7.64 (m, 1H), 7.10-6.99 (m, 3H), 6.99-6.91 (m, 1H), 6.88-6.83 (m, 1H), 3.25-3.16 (m, 2H), 3.13-3.00 (m 2H), 1.32 (s, 9H).

MS (ESI) m/z=331.1 [M−Boc+H]⁺, 475.3 [M+HCOO]⁻.

### tert-Butyl 4-(1-nitro-10H-phenothiazine-3-carbonyl)piperazine-1-carboxylate (13)

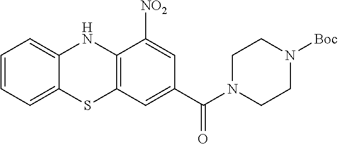 [SMILES: CC(C)(C)OC(=O)N1CCN(C(=O)c2cc3c(c([N+](=O)[O-])c2)Nc2ccccc2S3)CC1]

General procedure B was followed using compound 8 (0.384 g, 1.332 mmol), 1-Boc-piperazine as corresponding amine (0.347 g, 1.865 mmol), HATU (0.709 g, 1.865 mmol) and DIPEA (0.696 ml, 4.00 mmol). The reaction mixture was stirred over the weekend to provide 0.578 g (95%, crude yield) of compound 13.

¹H NMR (400 MHz, DMSO) δ 9.76 (s, 1H), 7.83 (d, J=1.9 Hz, 1H), 7.39-7.35 (m, 1H), 7.15-7.02 (m, 3H), 6.99 (d, J=7.6 Hz, 1H), 3.43-3.29 (m, 8H), 1.40 (s, 9H).

¹³C NMR (101 MHz, DMSO) δ 166.35, 153.81, 139.56, 137.46, 132.49, 130.55, 128.13, 127.77, 126.26, 125.02, 123.58, 121.33, 117.93, 116.86, 79.20, 28.02.

MS (ESI) m/z=455.2 [M−H]⁻.

### tert-Butyl 4-(2-(1-nitro-10H-phenothiazine-3-carboxamido)ethyl)piperazine-1-carboxylate (14)

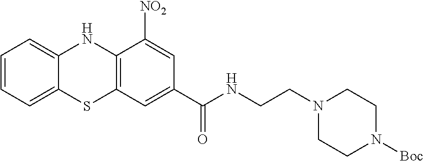 [SMILES: CC(C)(C)OC(=O)N1CCN(CCNC(=O)c2cc3c(c([N+](=O)[O-])c2)Nc2ccccc2S3)CC1]

General procedure B was followed using compound 8 (0.353 g, 1.225 mmol), 4-(2-aminoethyl)-1-Boc-piperazine as corresponding amine (0.393 g, 1.714 mmol), HATU (0.652 g, 1.714 mmol) and DIPEA (0.640 ml, 3.67 mmol). The reaction mixture was stirred over the weekend to provide 0.522 g (85%, crude yield) of compound 14.

¹H NMR (400 MHz, DMSO) δ 9.80 (s, 1H), 8.55 (t, J=5.6 Hz, 1H), 8.32 (d, J=2.0 Hz, 1H), 7.72 (s, 1H), 7.07 (dd, J=16.3, 5.9 Hz, 4H), 7.02-6.93 (m, 2H), 3.35 (d, J=6.8 Hz, 4H), 2.46 (t, J=6.9 Hz, 2H), 2.36 (t, J=4.9 Hz, 4H), 1.39 (s, 9H).

¹³C NMR (101 MHz, DMSO) δ 163.58, 154.27, 140.89, 137.64, 133.01, 130.41, 128.56, 126.83, 126.68, 125.58, 124.18, 121.27, 118.46, 117.27, 79.19, 57.26, 52.96, 37.31, 28.52.

MS (ESI) m/z=500.2 [M+H]⁺, 498.3 [M−H]⁻.

### (1-Amino-10H-phenothiazin-3-yl)(morpholino)methanone (15, CPD-005)

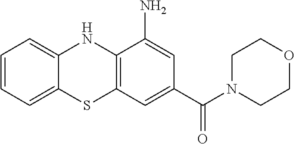 [SMILES: Nc1cc(C(=O)N2CCOCC2)cc2c1Nc1ccccc1S2]

General procedure C was followed using compound 9 (0.990 g, 2.77 mmol) and 20% Pd(OH)₂/C (50% of water, 0.134 g, 0.126 mmol). The reaction mixture was stirred overnight. The mixture was purified by reversed-phase column chromatography (10-65%. MeOH in water), providing 0.094 g of compound 15 (11%).

¹H NMR (400 MHz, DMSO) δ 7.71 (s, 1H), 7.01 (td, J=7.6, 1.5 Hz, 1H), 6.93 (dd, J=7.7, 1.4 Hz, 1H), 6.85 (dd, J=8.0, 1.3 Hz, 1H), 6.78 (td, J=7.5, 1.2 Hz, 1H), 6.51 (d, J=1.8 Hz, 1H), 6.28 (d, J=1.8 Hz, 1H), 5.24 (s, 2H), 3.59-3.52 (m, 4H), 3.47-3.43 (m, 4H).

¹³C NMR (101 MHz, DMSO) δ 168.86, 142.09, 134.55, 129.13, 128.92, 127.49, 126.19, 122.17, 116.94, 116.40, 115.04, 113.65, 113.22, 66.19.

MS (ESI) m/z 328.1 [M+H]⁺.

HRMS: calc 328.1114, found 328.1112 [M+H]⁺.

### 1-Amino-N-(2-morpholinoethyl)-10H-phenothiazine-3-carboxamide (16, CPD-006)

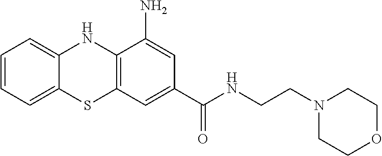 [SMILES: Nc1cc(C(=O)NCCN2CCOCC2)cc2c1Nc1ccccc1S2]

General procedure C was followed using compound 10 (0.500 g, 1.249 mmol) and 20% Pd(OH)₂/C (50% of water, 0.066 g, 0.062 mmol). The reaction mixture was stirred for 72 hours and purified by reversed-phase column chromatography (10-60% MeOH in water), yielding 0.337 g of compound 16 (73%).

¹H NMR (400 MHz, DMSO) δ 8.05 (t, J=5.7 Hz, 1H), 7.73 (s, 1H), 7.01 (td, J=7.6, 1.5 Hz, 1H), 6.97-6.91 (m, 2H), 6.87-6.82 (m, 1H), 6.82-6.71 (m, 2H), 5.19 (s, 2H), 3.55 (t, J=4.6 Hz, 4H), 3.29 (q, J=6.5 Hz, 2H), 2.44-2.35 (m, 6H).

¹³C NMR (101 MHz, DMSO) δ 165.58, 141.82, 134.32, 130.01, 128.42, 127.46, 126.15, 122.22, 116.88, 115.86, 115.08, 113.69, 113.51, 66.21, 57.46, 53.31, 36.40.

MS (ESI) m/z=371.2 [M+H]⁺.

HRMS: calc 371.1536, found 371.1523 [M+H]⁺.

### tert-Butyl(2-(1-amino-10H-phenothiazine-3-carboxamido)ethyl)(methyl)carbamate (17)

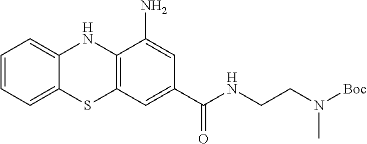 [SMILES: CN(CCNC(=O)c1cc(N)c2c(c1)Sc1ccccc1N2)C(=O)OC(C)(C)C]

General procedure C was followed using compound 11 (1.621 g, 3.65 mmol), 20% Pd(OH)₂/C (50% of water, 0.194 g, 0.182 mmol) was added and the reaction mixture was stirred for 72 hours. The mixture was purified by reversed-phase column chromatography (10-60% MeOH) and yielded 0.567 g of compound 17 (38%).

¹H NMR (400 MHz, DMSO) δ 8.21 (d, J=5.7 Hz, 1H), 7.74 (s, 1H), 7.03-6.96 (m, 2H), 6.93 (dd, J=7.7, 1.4 Hz, 1H), 6.85 (d, J=7.9 Hz, 1H), 6.81-6.70 (m, 2H), 3.28 (d, J=2.4 Hz, 4H), 2.77 (s, 3H), 1.30 (s, 9H).

¹³C NMR (101 MHz, DMSO) δ 165.76, 154.84, 141.84, 134.32, 130.07, 128.35, 127.46, 126.16, 122.24, 116.93, 115.83, 115.11, 113.82, 113.59, 78.28, 47.78, 37.38, 34.05, 28.03.

MS (ESI) m/z=315.2 [M+H]⁺.

### tert-Butyl (2-(1-amino-10H-phenothiazine-3-carboxamido)ethyl)carbamate (18)

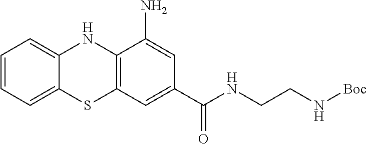 [SMILES: CC(C)(C)OC(=O)NCCNC(=O)c1cc(N)c2c(c1)Sc1ccccc1N2]

General procedure C was followed using compound 12 (1.73 g, 4.02 mmol), 20% Pd(OH)₂/C (50% of water, 214 g, 0.201 mmol) was added and the reaction mixture was stirred overnight. The mixture was purified by reversed-phase column chromatography (10-60% MeOH) and yielded 0.460 g of compound 18 (29%).

¹H NMR (400 MHz, MeOD) δ 6.95 (ddd, J=15.2, 7.7, 1.6 Hz, 2H), 6.88-6.80 (m, 2H), 6.75 (ddd, J=7.8, 5.5, 1.7 Hz, 2H), 3.35 (dd, J=12.3, 6.1 Hz, 2H), 3.29 (dt, J=3.3, 1.6 Hz, 2H), 3.21 (t, J=6.0 Hz, 2H), 1.41 (s, 9H).

¹³C NMR (101 MHz, MeOD) δ 169.93, 158.83, 142.95, 134.45, 133.96, 128.91, 128.40, 127.20, 123.59, 119.03, 118.71, 116.81, 116.10, 115.75, 80.25, 41.32, 40.89, 28.77.

MS (ESI) m/z=301.1 [M−Boc+H]⁺, 445.3 [M+HCOO]⁻.

### tert-Butyl 4-(1-amino-10H-phenothiazine-3-carbonyl)piperazine-1-carboxylate (19)

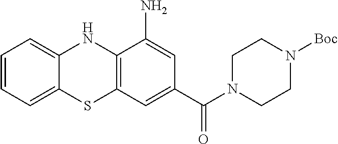 [SMILES: CC(C)(C)OC(=O)N1CCN(C(=O)c2cc(N)c3c(c2)Sc2ccccc2N3)CC1]

General procedure C was followed using compound 13 (0.587 g, 1.286 mmol), 20% Pd(OH)₂/C (50% of water, 0.014 g, 0.129 mmol) was added and the reaction mixture was stirred overnight. The mixture was purified by reversed-phase column chromatography (10-70% MeOH) and yielded 0.332 g of compound 19 (61%).

¹H NMR (400 MHz, DMSO) δ 7.71 (s, 1H), 7.01 (td, J=7.6, 1.5 Hz, 1H), 6.93 (dd, J=7.6, 1.4 Hz, 1H), 6.85 (dd, J=8.1, 1.3 Hz, 1H), 6.78 (td, J=7.5, 1.3 Hz, 1H), 6.51 (d, J=1.8 Hz, 1H), 6.28 (d, J=1.7 Hz, 1H), 5.24 (s, 2H), 3.44-3.39 (m, 4H), 3.35-3.29 (m, 4H), 1.40 (s, 9H).

¹³C NMR (101 MHz, DMSO) δ 168.96, 153.84, 142.09, 134.54, 129.23, 128.97, 127.49, 126.19, 122.17, 116.95, 116.39, 115.04, 113.66, 113.22, 79.18, 28.05.

MS (ESI) m/z=425.3 [M−H]⁻, 471.3 [M+HCOO]⁻.

### tert-Butyl 4-(2-(1-amino-10H-phenothiazine-3-carboxamido)ethyl)piperazine-1-carboxylate (20)

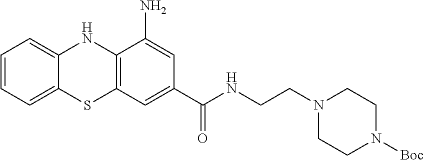 [SMILES: CC(C)(C)OC(=O)N1CCN(CCNC(=O)c2cc(N)c3c(c2)Sc2ccccc2N3)CC1]

General procedure C was followed using compound 14 (0.522 g, 1.045 mmol), 20% Pd(OH)2/C (50% of water, 0.056 g, 0.522 mmol) was added and the reaction mixture was stirred overnight. The mixture was purified by reversed-phase column chromatography (20-70% MeOH) and yielded 0.406 g of compound 20 (83%).

¹H NMR (400 MHz, DMSO) δ 8.05 (t, J=5.6 Hz, 1H), 7.75 (s, 1H), 7.05-6.90 (m, 3H), 6.85 (d, J=7.9 Hz, 1H), 6.80-6.73 (m, 2H), 5.20 (s, 2H), 3.30 (q, J=6.4 Hz, 6H), 2.42 (t, J=7.0 Hz, 2H), 2.34 (t, J=5.0 Hz, 4H), 1.38 (s, 9H).

¹³C NMR (101 MHz, DMSO) δ 165.62, 153.83, 141.82, 134.31, 130.04, 128.41, 127.43, 126.13, 122.20, 116.89, 115.88, 115.08, 113.68, 113.53, 78.73, 56.96, 52.50, 36.61, 28.07.

MS (ESI) m/z=470.3 [M+H]⁺, 514.3 [M+HCOO]⁻.

### 1-Amino-N-(2-(methylamino)ethyl)-10H-phenothiazine-3-carboxamide trihydrochloride (21, CPD-007)

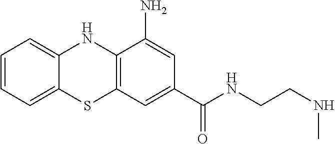 [SMILES: CNCCNC(=O)c1cc(N)c2c(c1)Sc1ccccc1N2]

General procedure E was followed using compound 17 (0.120 g, 0,289 mmol) and 4M HCl in dioxane (1.09 ml, 4.34 mmol) to afford 0.080 g of the desired compound 21 (65%). The reported NMR spectrum refers to trihydrochloride salt.

¹H NMR (400 MHz, DMSO) δ 8.94 (d, J=9.0 Hz, 2H), 8.86 (s, 1H), 8.66 (t, J=5.5 Hz, 1H), 7.43-7.38 (m, 2H), 7.12-7.00 (m, 2H), 6.96 (d, J=7.5 Hz, 1H), 6.84 (t, J=7.4 Hz, 1H), 3.51 (q, J=5.8 Hz, 2H), 3.04 (p, J=6.0 Hz, 2H), 2.55 (t, J=5.4 Hz, 3H).

¹³C NMR (101 MHz, DMSO) δ 165.19, 140.49, 135.71, 127.72, 127.30, 126.18, 123.16, 121.00, 120.34, 117.63, 116.34, 115.76, 48.61, 47.85, 35.69, 32.46.

MS (ESI) m/z=315.1 [M+H]⁺, 313.1 [M−H]⁻.

HRMS: calc 315, 12741, found 315, 1284 [M+H]⁺.

### 1-Amino-N-(2-aminoethyl)-10H-phenothiazine-3-carboxamide trihydrochloride (22, CPD-008)

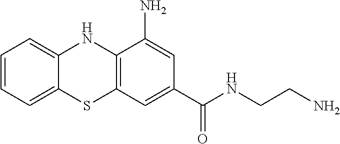 [SMILES: NCCNC(=O)c1cc(N)c2c(c1)Sc1ccccc1N2]

General procedure E was followed using compound 18 (0.037 g, 0.091 mmol) and 4M HCl in dioxane (0.343 ml, 1.37 mmol) to afford 0.030 g of the desired compound 22 (81%). The reported NMR spectrum refers to trihydrochloride salt.

¹H NMR (400 MHz, MeOD) δ 7.63 (s, 1H), 7.53 (d, J=8.1 Hz, 1H), 7.10-7.00 (m, 1H), 6.92 (s, 3H), 3.64 (t, J=5.8 Hz, 2H), 3.16 (t, J=5.9 Hz, 2H).

¹³C NMR (101 MHz, MeOD) δ 169.97, 142.96, 134.44, 133.96, 128.84, 128.44, 127.13, 123.57, 118.89, 118.70, 116.95, 116.25, 115.62, 43.53, 42.10.

MS (ESI) m/z=301.1 [M+H]⁺, 299.1 [M−H]⁻.

HRMS: calc 301.1118, found 301.1124 [M+H]⁺.

### (1-Amino-10H-phenothiazin-3-yl)(piperazin-1-yl)methanone trihydrochloride (23, CPD-009)

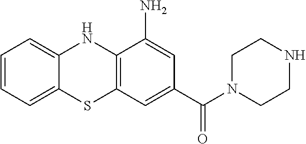 [SMILES: Nc1cc(C(=O)N2CCNCC2)cc2c1Nc1ccccc1S2]

General procedure E was followed using compound 19 (0.089 g, 0.209 mmol) and 4M HCl in dioxane (0.782 ml, 3.13 mmol) to afford 0.087 g of the desired compound 23 (96%). The reported NMR spectrum refers to trihydrochloride salt.

¹H NMR (400 MHz, DMSO) δ 9.52 (s, 2H), 8.77 (s, 1H), 7.10-6.99 (m, 3H), 6.96 (d, J=7.5 Hz, 1H), 6.84 (d, J=6.9 Hz, 2H), 3.72-3.67 (m, 4H), 3.13-3.07 (m, 4H).

¹³C NMR (101 MHz, DMSO) δ 167.93, 140.76, 134.74, 127.85, 127.73, 126.21, 123.39, 123.05, 121.21, 119.73, 118.24, 116.43, 115.72, 48.61, 42.48.

MS (ESI) m/z=327.1 [M+H]⁺, 325.1 [M−H]⁻, 371.2 [M+HCOO]⁻.

HRMS: calc 327.1274, found 327.1259 [M+H]⁺.

### 1-Amino-N-(2-(piperazin-1-yl)ethyl)-10H-phenothiazine-3-carboxamide tetrahydrochloride (24, CPD-010)

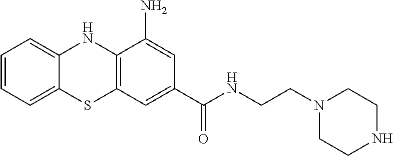 [SMILES: Nc1cc(C(=O)NCCN2CCNCC2)cc2c1Nc1ccccc1S2]

General procedure E was followed using compound 20 (0.091 g, 0.194 mmol) and 4M HCl in dioxane (0.727 ml, 2.91 mmol) to afford 0.077 g of the desired compound 24 (83%). The reported NMR spectrum refers to tetrahydrochloride salt.

¹H NMR (400 MHz, DMSO) δ 9.78 (s, 2H), 8.63 (dd, J=11.6, 5.9 Hz, 2H), 7.33-7.24 (m, 2H), 7.03 (d, J=4.2 Hz, 2H), 6.95 (d, J=7.6 Hz, 1H), 6.83 (dq, J=8.2, 4.3 Hz, 1H), 3.85-3.25 (m, 12H).

¹³C NMR (101 MHz, DMSO) δ 165.31, 140.80, 134.30, 127.62, 127.20, 126.14, 122.90, 118.93, 118.42, 117.19, 116.46, 115.57, 55.19, 47.99, 33.83.

MS (ESI) m/z=370.2 [M+H]1, 368.1 [M−H]⁻.

### (1-(Benzylamino)-10H-phenothiazin-3-yl)(morpholino)methanone (25, CPD-011)

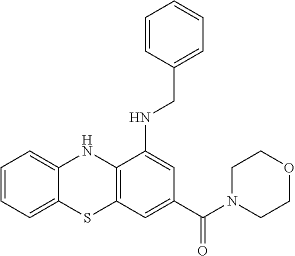 [SMILES: O=C(c1cc(NCc2ccccc2)c2c(c1)Sc1ccccc1N2)N1CCOCC1]

General procedure D was followed using compound 15 (0.074 g, 0.225 mmol), acetic acid (0.296 ml, 5.18 mmol), benzaldehyde as corresponding aldehyde (0.069 ml, 0.675 mmol) and sodium cyanoborohydride (0.035 g, 0.563 mmol). The reaction mixture was stirred overnight. Purification of the obtained mixture by reversed-phase column chromatography (10-70% MeOH) resulted in 0.019 g of compound 25 (20%).

¹H NMR (400 MHz, DMSO) δ 7.96 (s, 1H), 7.35 (d, J=4.4 Hz, 4H), 7.25 (h, J=3.9 Hz, 1H), 7.04 (td, J=7.6, 1.6 Hz, 1H), 6.96 (dd, J=7.7, 1.4 Hz, 1H), 6.89 (d, J=7.8 Hz, 1H), 6.81 (t, J=7.5 Hz, 1H), 6.32 (d, J=1.6 Hz, 1H), 6.23 (d, J=1.7 Hz, 1H), 6.09 (t, J=5.6 Hz, 1H), 4.39 (d, J=5.3 Hz, 2H), 3.62-3.11 (m, 8H).

¹³C NMR (101 MHz, DMSO) δ 168.87, 142.06, 139.25, 134.20, 129.63, 128.91, 128.51, 127.58, 127.10, 126.89, 126.22, 122.49, 117.19, 116.25, 115.27, 114.21, 109.32, 66.01, 46.48.

MS (ESI) m/z=418.3 [M+H]⁺.

HRMS: calc 418.1584, found 418.1599 [M+H]⁺.

### (1-((4-Fluorobenzyl)amino)-10H-phenothiazin-3-yl)(morpholino)methanone (26, CPD-012)

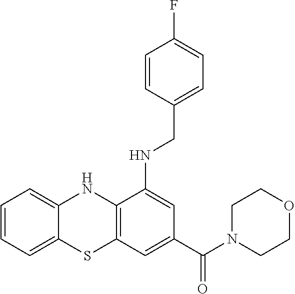 [SMILES: O=C(c1cc(NCc2ccc(F)cc2)c2c(c1)Sc1ccccc1N2)N1CCOCC1]

General procedure D was followed using compound 15 (0.909 g, 2.78 mmol) acetic acid (3.65 ml, 63.8 mmol), 4-fluorobenzaldehyde as corresponding aldehyde (0.893 ml, 8.33 mmol) and sodium cyanoborohydride (0.436 g, 6.94 mmol). The reaction mixture was stirred overnight and purified by reversed-phase column chromatography (10-80% MeOH in water). This resulted in 0.507 g of compound 26 (42%).

¹H NMR (400 MHz, DMSO) δ 7.94 (s, 1H), 7.39 (dd, J=8.4, 5.5 Hz, 2H), 7.17 (t, J=8.9 Hz, 2H), 7.03 (td, J=7.6, 1.5 Hz, 1H), 6.95 (dd, J=7.7, 1.4 Hz, 1H), 6.89 (dd, J=8.0, 1.2 Hz, 1H), 6.81 (td, J=7.4, 1.2 Hz, 1H), 6.34 (d, J=1.6 Hz, 1H), 6.24 (d, J=1.7 Hz, 1H), 6.06 (t, J=5.5 Hz, 1H), 4.37 (d, J=5.2 Hz, 2H), 3.60-3.09 (m, 8H).

¹³C NMR (101 MHz, DMSO) δ 168.87, 161.26 (d, J=242.4 Hz), 142.03, 135.34, 135.31, 134.10, 129.69, 129.05 (d, J=8.1 Hz), 128.97, 127.54, 126.20, 122.47, 117.20, 116.30, 115.19 (d, J=21.2 Hz), 114.77 (d, J=98.6 Hz), 109.31, 66.01, 45.78.

MS (ESI) m/z 436.2 [M+H]³⁰.

HRMS: calc 436.1490, found 436.1507 [M+H]⁺.

### 1-(Benzylamino)-N-(2-morpholinoethyl)-10H-phenothiazine-3-carboxamide (27, CPD-013)

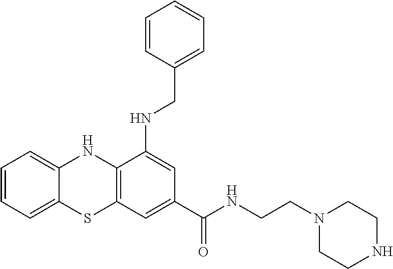 [SMILES: O=C(NCCN1CCNCC1)c1cc(NCc2ccccc2)c2c(c1)Sc1ccccc1N2]

General procedure D was followed using compound 16 (0.154 g, 0.415 mmol), acetic acid (0.546 ml, 9.55 mmol), benzaldehyde as corresponding aldehyde (0.127 ml, 1.246 mmol) and sodium cyanoborohydride (0.065 g, 1.039 mmol). The reaction mixture was stirred overnight and purified by reversed-phase column chromatography (10-60% MeOH in water). The reaction yielded 0.034 g of compound 27 (18%).

¹H NMR (400 MHz, DMSO) δ 8.10 (t, J=5.7 Hz, 1H), 7.99 (s, 1H), 7.42-7.31 (m, 4H), 7.26 (t, J=7.1 Hz, 1H), 7.02 (t, J=7.7 Hz, 1H), 6.95 (d, J=7.7 Hz, 1H), 6.91-6.85 (m, 2H), 6.84-6.76 (m, 2H), 5.90 (t, J=5.5 Hz, 1H), 4.38 (d, J=5.4 Hz, 2H), 3.57-3.50 (m, 4H), 3.28 (q, J=6.8 Hz, 2H), 2.42-2.33 (m, 6H).

¹³C NMR (101 MHz, DMSO) δ 165.57, 141.79, 139.34, 134.55, 130.76, 128.47, 128.24, 127.52, 127.50, 127.00, 126.17, 122.53, 117.16, 115.63, 115.31, 114.04, 109.28, 66.21, 57.47, 53.32, 46.88, 36.46.

MS (ESI) m/z 461.2 [M+H]⁺.

HRMS: calc 461.2006, found 461, 1991 [M+H]⁺.

### 1-((4-Fluorobenzyl)amino)-N-(2-morpholinoethyl)-10H-phenothiazine-3-carboxamide (28, CPD-014)

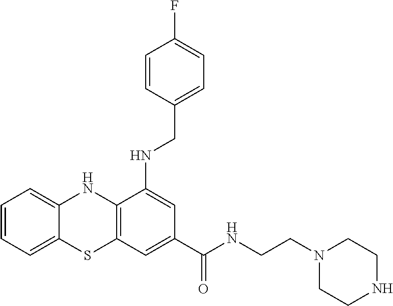 [SMILES: O=C(NCCN1CCNCC1)c1cc(NCc2ccc(F)cc2)c2c(c1)Sc1ccccc1N2]

General procedure D was followed using compound 16 (0.136 g, 0.368 mmol), acetic acid (0.484 ml, 8.47 mmol), 4-fluorobenzaldehyde as corresponding aldehyde (0.118 ml, 1.104 mmol) and sodium cyanoborohydride (0.058 g, 0.920 mmol). The reaction mixture was stirred overnight and subsequently purified using reversed-phase column chromatography (10-60% MeOH in water). The reaction provided 0.028 g of compound 28 (16%).

¹H NMR (400 MHz, DMSO) δ 8.09 (t, J=5.7 Hz, 1H), 7.95 (s, 1H), 7.42 (dd, J=8.4, 5.6 Hz, 2H), 7.18 (t, J=8.7 Hz, 2H), 7.02 (t, J=7.6 Hz, 1H), 6.95 (d, J=7.6 Hz, 1H), 6.89-6.78 (m, 4H), 5.86 (t, J=5.5 Hz, 1H), 4.36 (d, J=5.3 Hz, 2H), 3.54 (t, J=4.7 Hz, 4H), 3.28 (q, J=6.6 Hz, 2H), 2.37 (q, J=6.1 Hz, 6H).

¹³C NMR (101 MHz, DMSO) δ 165.48, 161.31 (d, J=242.2 Hz), 141.74, 135.43 (d, J=2.8 Hz), 134.38, 130.80, 129.37 (d, J=8.1 Hz), 128.22, 127.52, 126.17, 122.53, 117.13, 115.64, 115.29, 115.08, 114.13, 109.31, 66.21, 57.48, 53.32, 46.13, 36.46.

MS (ESI) m/z=479.3 [M+H]⁺.

HRMS: calc 479, 1911, found 479, 1928 [M+H]⁺.

### 1-(Cyclohexylamino)-N-(2-morpholinoethyl)-10H-phenothiazine-3-carboxamide (29, CPD-015)

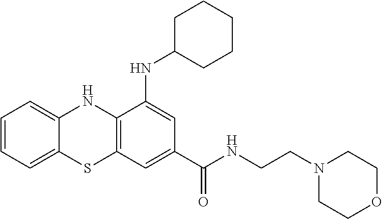 [SMILES: O=C(NCCN1CCOCC1)c1cc(NC2CCCCC2)c2c(c1)Sc1ccccc1N2]

General procedure D was followed using compound 16 (0.075 g, 0.202 mmol), acetic acid (0.266 ml, 4.66 mmol), cyclohexanone as corresponding ketone (0.063 ml, 0.607 mmol) and sodium cyanoborohydride (0.032 g, 0.506 mmol). The reaction mixture was stirred overnight and purified twice by reversed-phase column chromatography (10-80% MeOH in water). The reaction yielded 0.005 g of compound 29 (6%).

¹H NMR (400 MHz, DMSO) δ 8.11 (t, J=5.7 Hz, 1H), 7.92 (s, 1H), 7.05-6.99 (m, 1H), 6.94 (dd, J=7.7, 1.4 Hz, 1H), 6.88 (dt, J=4.3, 1.7 Hz, 2H), 6.82-6.75 (m, 2H), 5.00 (d, J=7.2 Hz, 1H), 3.56 (t, J=4.6 Hz, 4H), 3.33-3.25 (m, 2H), 2.45-2.36 (m, 6H), 2.03-1.95 (m, 2H), 1.77 (d, J=12.9 Hz, 2H), 1.65 (d, J=12.5 Hz, 1H), 1.43-1.29 (m, 2H), 1.26-1.14 (m, 4H).

¹³C NMR (101 MHz, DMSO) δ 165.67, 141.88, 133.73, 130.62, 128.35, 127.44, 126.13, 122.41, 117.26, 115.87, 115.25, 113.46, 109.36, 66.24, 57.43, 53.33, 51.34, 36.50, 32.80, 25.63, 24.89.

MS (ESI) m/z=453.2 [M+H]⁺.

### 1-(Cyclopentylamino)-N-(2-morpholinoethyl)-10H-phenothiazine-3-carboxamide (30, CPD-016)

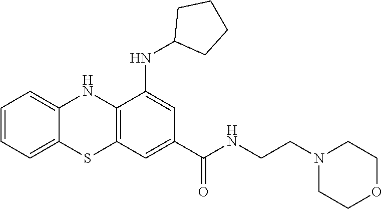 [SMILES: O=C(NCCN1CCOCC1)c1cc(NC2CCCC2)c2c(c1)Sc1ccccc1N2]

General procedure D was followed using compound 16 (0.075 g, 0.202 mmol), acetic acid (0.266 ml, 4.66 mmol), cyclopentanone as corresponding ketone (0.054 ml, 0.607 mmol) and sodium cyanoborohydride (0.032 g, 0.506 mmol). The reaction mixture was stirred overnight and purified twice by reversed-phase column chromatography (10-80% MeOH in water). The reaction yielded 0.015 g of compound 30 (17%).

¹H NMR (400 MHz, DMSO) δ 8.05 (t, J=5.8 Hz, 1H), 7.84 (s, 1H), 6.94 (t, J=8.1 Hz, 1H), 6.87 (d, J=7.5 Hz, 1H), 6.83-6.69 (m, 4H), 5.07 (d, J=5.9 Hz, 1H), 3.72 (q, J=6.3 Hz, 1H), 3.48 (t, J=4.6 Hz, 4H), 3.24 (d, J=6.6 Hz, 2H), 2.33 (q, J=6.3 Hz, 6H), 1.92 (dt, J=12.9, 6.3 Hz, 2H), 1.63 (d, J=8.0 Hz, 2H), 1.57-1.10 (m, 4H).

¹³C NMR (101 MHz, DMSO) δ 165.71, 141.86, 134.47, 130.68, 128.32, 127.51, 126.21, 122.49, 117.30, 115.55, 115.29, 113.63, 109.58, 66.26, 57.49, 54.12, 53.37, 36.51, 32.58, 23.98.

MS (ESI) m/z=439.4 [M+H]⁺.

### tert-Butyl N-[2-[[1-[(phenyl)methylamino]-10H-phenothiazine-3-carbonyl]amino]ethyl]-N-methyl-carbamate (31)

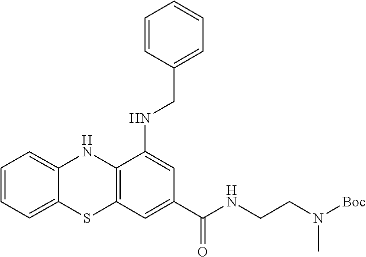 [SMILES: CN(CCNC(=O)c1cc(NCc2ccccc2)c2c(c1)Sc1ccccc1N2)C(=O)OC(C)(C)C]

General procedure D was followed using compound 17 (0.255 g, 0,615 mmol) acetic acid (0.809 ml, 14.15 mmol), benzaldehyde as corresponding aldehyde (0.196 ml, 1.845 mmol) and sodium cyanoborohydride (0.097 g, 1.538 mmol). The reaction mixture was stirred overnight and purified twice by reversed-phase column chromatography (10-80% MeOH in water). This resulted in 0.120 g of compound 31 (39%).

¹H NMR (400 MHz, DMSO) δ 8.26-8.16 (m, 1H), 7.97 (s, 1H), 7.42-7.31 (m, 4H), 7.31-7.22 (m, 1H), 7.02 (td, J=7.6, 1.5 Hz, 1H), 6.95 (dd, J=7.7, 1.4 Hz, 1H), 6.91-6.75 (m, 4H), 5.90-5.83 (m, 1H), 4.37 (d, J=5.3 Hz, 2H), 3.26 (d, J=3.0 Hz, 4H), 2.75 (d, J=8.8 Hz, 3H), 1.39-1.26 (m, 9H).

¹³C NMR (101 MHz, DMSO) δ 165.60, 154.77, 141.75, 139.29, 135.84, 134.51, 130.69, 128.43, 128.17, 127.47, 126.96, 126.13, 122.48, 117.12, 115.49, 115.27, 114.04, 109.29, 78.24, 47.75, 46.89, 37.42, 34.04, 28.00.

MS (ESI) m/z=405.3 [M−Boc+H]⁺, 549.3 [M+HCOO]⁻.

### tert-Butyl N-[2-[[1-[(4-fluorophenyl)methylamino]-10H-phenothiazine-3-carbonyl]amino]ethyl]-N-methyl-carbamate (32)

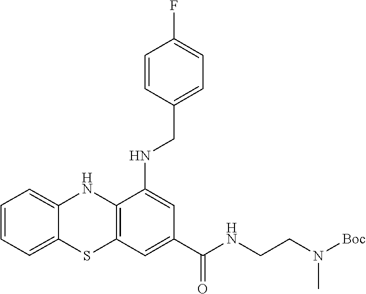 [SMILES: CN(CCNC(=O)c1cc(NCc2ccc(F)cc2)c2c(c1)Sc1ccccc1N2)C(=O)OC(C)(C)C]

General procedure D was followed using compound 17 (0.255 g, 0,615 mmol) acetic acid (0.809 ml, 14.15 mmol), 4-fluorobenzaldehyde as corresponding aldehyde (0.198 ml, 1.845 mmol) and sodium cyanoborohydride (0.097 g, 1.538 mmol). The reaction mixture was stirred overnight and purified twice by reversed-phase column chromatography (10-80% MeOH in water). This resulted in 0.171 g of compound 32 (53%).

¹H NMR (400 MHz, DMSO) δ 8.24 (s, 1H), 7.95 (s, 1H), 7.42 (dd, J=8.4, 5.6 Hz, 2H), 7.23-7.13 (m, 2H), 7.02 (td, J=7.6, 1.5 Hz, 1H), 6.95 (dd, J=7.7, 1.4 Hz, 1H), 6.90-6.76 (m, 4H), 5.86 (t, J=5.5 Hz, 1H), 4.36 (d, J=5.3 Hz, 2H), 3.26 (d, J=3.0 Hz, 4H), 2.75 (d, J=8.0 Hz, 3H), 1.36 (s, 3H), 1.28 (s, 6H).

¹³C NMR (101 MHz, DMSO) δ 165.58, 161.29 (d, J=242.3 Hz), 154.77, 141.71, 135.39 (d, J=2.9 Hz), 134.34, 130.77, 129.33 (d, J=8.1 Hz), 128.16, 127.48, 126.13, 122.49, 117.10, 115.53, 115.28, 115.15 (d, J=21.3 Hz), 114.15, 109.35, 78.36 (d, J=25.5 Hz), 47.56 (d, J=38.2 Hz), 46.12, 37.42, 34.29 (d, J=54.7 Hz) 27.99.

MS (ESI) m/z=423.3 [M−Boc+H]⁺, 567.3 [M+HCOO]⁻.

### tert-Butyl (2-(1-(cyclohexylamino)-10H-phenothiazine-3-carboxamido)ethyl)(methyl)carbamate (33)

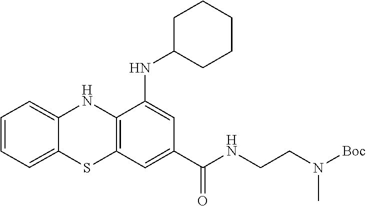 [SMILES: CN(CCNC(=O)c1cc(NC2CCCCC2)c2c(c1)Sc1ccccc1N2)C(=O)OC(C)(C)C]

General procedure D was followed using compound 17 (0.150 g, 0,362 mmol) acetic acid (0.476 ml, 8.32 mmol), cyclohexanone as corresponding ketone (0.112 ml, 1.086 mmol) and sodium cyanoborohydride (0.057 g, 0.905 mmol). The reaction mixture was stirred overnight and purified twice by reversed-phase column chromatography (10-80% MeOH in water). This resulted in 0.116 g of compound 33 (65%).

¹H NMR (400 MHz, DMSO) δ 8.29-8.15 (m, 1H), 7.90 (s, 1H), 7.05-6.99 (m, 1H), 6.94 (dd, J=7.7, 1.4 Hz, 1H), 6.93-6.85 (m, 2H), 6.83-6.73 (m, 2H), 4.97 (d, J=7.2 Hz, 1H), 3.30 (d, J=3.4 Hz, 6H), 2.79 (d, J=10.0 Hz, 4H), 2.00 (d, J=12.0 Hz, 2H), 1.77 (d, J=12.8 Hz, 2H), 1.65 (d, J=12.5 Hz, 1H), 1.37 (s, 3H), 1.30 (s, 6H), 1.27-1.13 (m, 3H).

¹³C NMR (101 MHz, DMSO) δ 165.83, 154.81, 141.86, 133.69, 130.65, 128.29, 127.42, 126.12, 122.39, 117.27, 115.79, 115.24, 113.53, 109.60, 78.23, 51.38, 47.73, 37.41, 33.95, 32.80, 28.01, 25.62, 24.88.

MS (ESI) m/z=397.3 [M−Boc+H]⁺, 541.4 [M+HCOO]⁻.

### tert-Butyl (2-(1-(cyclopentylamino)-10H-phenothiazine-3-carboxamido)ethyl)(methyl)carbamate (34)

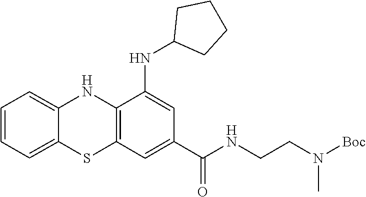 [SMILES: CN(CCNC(=O)c1cc(NC2CCCC2)c2c(c1)Sc1ccccc1N2)C(=O)OC(C)(C)C]

General procedure D was followed using compound 17 (0.150 g, 0,362 mmol) acetic acid (0.476 ml, 8.32 mmol), cyclohexanone as corresponding ketone (0.096 ml, 1.086 mmol) and sodium cyanoborohydride (0.057 g, 0.905 mmol). The reaction mixture was stirred overnight and purified by reversed-phase column chromatography (20-100% MeOH in water). This resulted in 0.079 g of compound 34 (45%).

¹H NMR (400 MHz, DMSO) δ 8.28-8.15 (m, 1H), 7.88 (s, 1H), 7.00 (td, J=7.6, 1.5 Hz, 1H), 6.95-6.83 (m, 3H), 6.82-6.74 (m, 2H), 5.10 (d, J=5.9 Hz, 1H), 3.77 (h, J=6.1 Hz, 1H), 3.30-3.20 (m, 4H), 2.78 (d, J=11.0 Hz, 3H), 1.97 (dt, J=13.1, 6.5 Hz, 2H), 1.70 (dd, J=9.8, 5.7 Hz, 2H), 1.65-1.41 (m, 4H), 1.35 (s, 3H), 1.28 (s, 6H).

¹³C NMR (101 MHz, DMSO) δ 165.75, 154.80, 141.81, 134.38, 130.63, 128.22, 127.42, 126.13, 122.40, 117.27, 115.41, 115.22, 113.63, 109.70, 78.21, 54.08, 47.75, 37.41, 33.97, 32.54, 28.00, 23.92.

MS (ESI) m/z=383.3 [M−Boc+H]⁺.

### tert-Butyl (2-(1-(benzylamino)-10H-phenothiazine-3-carboxamido)ethyl)carbamate (35)

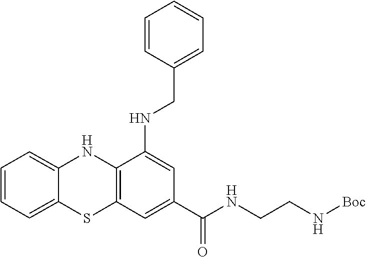 [SMILES: CC(C)(C)OC(=O)NCCNC(=O)c1cc(NCc2ccccc2)c2c(c1)Sc1ccccc1N2]

General procedure D was followed using compound 18 (0.160 g, 0.400 mmol) acetic acid (0.526 ml, 9.19 mmol), benzaldehyde as corresponding aldehyde (0.122 ml, 1.199 mmol) and sodium cyanoborohydride (0.063 g, 0.999 mmol). The reaction mixture was stirred overnight and purified twice by reversed-phase column chromatography (10-80% MeOH in water). This resulted in 0.122 g of compound 35 (62%).

¹H NMR (400 MHz, DMSO) δ 8.15 (t, J=5.6 Hz, 1H), 7.97 (s, 1H), 7.43-7.32 (m, 4H), 7.31-7.22 (m, 1H), 7.06-6.92 (m, 2H), 6.91-6.76 (m, 5H), 5.86 (t, J=5.5 Hz, 1H), 4.37 (d, J=5.4 Hz, 2H), 3.18 (dd, J=6.8, 5.2 Hz, 2H), 3.03 (q, J=6.3 Hz, 2H), 1.37 (s, 9H).

¹³C NMR (101 MHz, DMSO) δ 165.71, 155.74, 141.75, 139.29, 134.52, 130.73, 128.45, 128.15, 127.54, 127.48, 126.99, 126.13, 122.49, 117.14, 115.53, 115.27, 114.09, 109.28, 77.68, 46.94, 28.25.

MS (ESI) m/z=511.2 [M+Na]⁺, 535.3 [M+HCOO]⁻.

### tert-Butyl (2-(1-((4-fluorobenzyl)amino)-10H-phenothiazine-3-carboxamido)ethyl)carbamate (36)

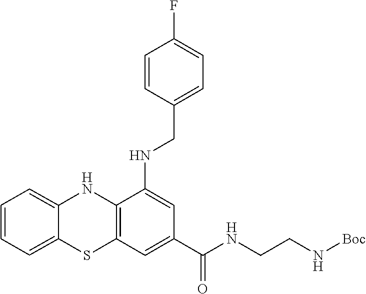 [SMILES: CC(C)(C)OC(=O)NCCNC(=O)c1cc(NCc2ccc(F)cc2)c2c(c1)Sc1ccccc1N2]

General procedure D was followed using compound 18 (0.140 g, 0.350 mmol), acetic acid (0.46 ml, 8.04 mmol), 4-fluorobenzaldehyde as corresponding aldehyde (0.112 ml, 1.199 mmol) and sodium cyanoborohydride (0.055 g, 0.847 mmol). The reaction mixture was stirred overnight and purified twice by reversed-phase column chromatography (10-80% MeOH in water). This resulted in 0.057 g of compound 36 (32%).

¹H NMR (400 MHz, DMSO) δ 8.14 (t, J=5.6 Hz, 1H), 7.94 (s, 1H), 7.43 (dd, J=8.5, 5.7 Hz, 2H), 7.19 (t, J=8.8 Hz, 2H), 7.02 (t, J=7.6 Hz, 1H), 6.98-6.92 (m, 1H), 6.91-6.76 (m, 5H), 5.84 (t, J=5.4 Hz, 1H), 4.35 (d, J=5.3 Hz, 2H), 3.18 (q, J=6.2 Hz, 2H), 3.07-2.99 (m, 2H), 1.36 (s, 9H).

MS (ESI) m/z=531.3 [M+Na]⁺, 553.3 [M+HCOO]⁻.

### tert-Butyl (2-(1-((2,4-difluorobenzyl)amino)-10H-phenothiazine-3-carboxamido)ethyl) carbamate (37)

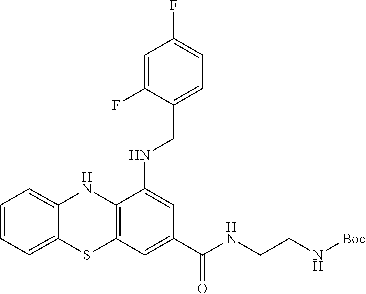 [SMILES: CC(C)(C)OC(=O)NCCNC(=O)c1cc(NCc2ccc(F)cc2F)c2c(c1)Sc1ccccc1N2]

General procedure D was followed using compound 18 (0.158 g, 0.395 mmol), acetic acid (0.52 ml, 9.07 mmol), 2,4-difluorobenzaldehyde as corresponding aldehyde (0.129 ml, 1.184 mmol) and sodium cyanoborohydride (0.062 g, 0.986 mmol). The reaction mixture was stirred overnight and purified twice by reversed-phase column chromatography (10-80% MeOH in water). This resulted in 0.091 g of compound 37 (44%).

¹H NMR (400 MHz, MeOD) δ 7.44 (td, J=8.5, 6.4 Hz, 1H), 7.06-6.86 (m, 6H), 6.85-6.69 (m, 3H), 4.65 (s, 1H), 4.42 (d, J=10.2 Hz, 2H), 3.36 (q, J=5.8 Hz, 2H), 3.22 (p, J=5.5 Hz, 2H), 1.42 (d, J=2.3 Hz, 9H).

¹³C NMR (101 MHz, MeOD) δ 157.50, 141.53, 134.05, 133.89, 132.88, 132.75, 127.40, 127.05, 125.82, 122.43, 122.38, 117.87, 117.82, 116.97, 116.87, 115.37, 115.24, 114.86, 114.78, 110.10, 109.62, 103.20, 101.73, 78.84, 40.06, 39.47, 27.36.

MS (ESI) m/z=549.2 [M+Na]⁺, 571.2 [M+HCOO]⁻.

### tert-Butyl (2-(1-((3,5-difluorobenzyl)amino)-10H-phenothiazine-3-carboxamido)ethyl) carbamate (38)

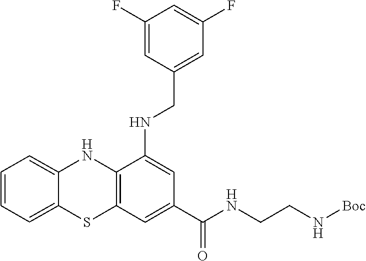 [SMILES: CC(C)(C)OC(=O)NCCNC(=O)c1cc(NCc2cc(F)cc(F)c2)c2c(c1)Sc1ccccc1N2]

General procedure D was followed using compound 18 (0.158 g, 0.395 mmol), acetic acid (0.52 ml, 9.07 mmol), 3,5-difluorobenzaldehyde as corresponding aldehyde (0.130 ml, 1.184 mmol) and sodium cyanoborohydride ride (0.062 g, 0.986 mmol). The reaction mixture was stirred overnight and purified twice by reversed-phase column chromatography (10-80% MeOH in water). This resulted in 0.065 g of compound 38 (31%).

¹H NMR (400 MHz, MeOD) δ 8.24 (dd, J=12.1, 6.2 Hz, 1H), 7.44 (td, J=8.5, 6.4 Hz, 1H), 7.06-6.95 (m, 3H), 6.94-6.69 (m, 5H), 4.65 (s, 1H), 4.42 (d, J=10.1 Hz, 2H), 3.37 (p, J=6.1 Hz, 2H), 3.21 (q, J=6.0 Hz, 2H), 1.41 (d, J=2.3 Hz, 9H)

¹³C NMR (101 MHz, MeOD) δ 168.53, 159.86, 157.50, 141.52, 134.04, 132.88, 130.81, 127.39, 127.01, 125.80, 122.38, 117.82, 116.86, 115.37, 114.86, 114.78, 110.89, 110.68, 110.10, 103.19, 102.93, 101.73, 78.85, 41.07, 40.06, 39.48, 27.37.

MS (ESI) m/z=549.2 [M+Na]⁺, 571.3 [M+HCOO]⁻.

### tert-Butyl (2-(1-((4-(trifluoromethyl)benzyl)amino)-10H-phenothiazine-3-carboxamido)ethyl) carbamate (39)

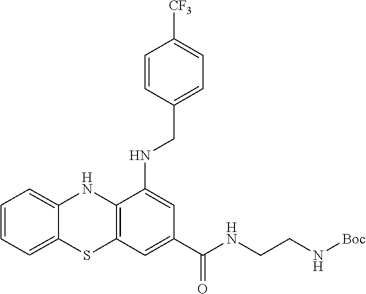 [SMILES: CC(C)(C)OC(=O)NCCNC(=O)c1cc(NCc2ccc(C(F)(F)F)cc2)c2c(c1)Sc1ccccc1N2]

General procedure D was followed using compound 18 (0.158 g, 0.395 mmol), acetic acid (0.52 ml, 9.07 mmol), 4-(trifluoromethyl)benzaldehyde as corresponding aldehyde (0.162 ml, 1.184 mmol) and sodium cyanoborohydride ride (0.062 g, 0.986 mmol). The reaction mixture was stirred overnight and purified twice by reversed-phase column chromatography (10-80% MeOH in water). This resulted in 0.068 g of compound 39 (31%).

¹H NMR (400 MHz, MeOD) δ 7.70-7.57 (m, 4H), 6.98 (td, J=7.6, 1.5 Hz, 1H), 6.89 (ddd, J=13.1, 5.9, 1.9 Hz, 3H), 6.83-6.74 (m, 2H), 4.51 (s, 2H), 3.35 (t, J=6.1 Hz, 2H), 3.20 (t, J=6.0 Hz. 2H). 1.41 (s. 9H).

MS (ESI) m/z=581.2 [M+Na]⁺, 603.3 [M+HCOO]⁻.

### tert-Butyl 4-(1-(benzylamino)-10H-phenothiazine-3-carbonyl)piperazine-1-carboxylate (40)

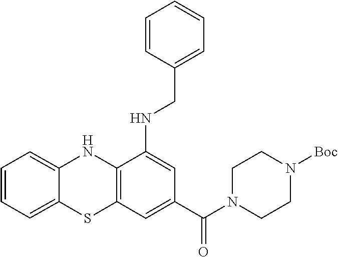 [SMILES: CC(C)(C)OC(=O)N1CCN(C(=O)c2cc(NCc3ccccc3)c3c(c2)Sc2ccccc2N3)CC1]

General procedure D was followed using compound 19 (0.122 g, 0.286 mmol), acetic acid (0.38 ml, 6.58 mmol), benzaldehyde as corresponding aldehyde (0.088 ml, 0.858 mmol) and sodium cyanoborohydride (0.045 g, 0.715 mmol). The reaction mixture was stirred overnight and purified by reversed-phase column chromatography (10-80% MeOH in water). This resulted in 0.101 g of compound 40 (68%).

¹H NMR (400 MHz, DMSO) δ 7.95 (s, 1H), 7.39-7.30 (m, 4H), 7.24 (tt, J=5.8, 3.1 Hz, 1H), 7.04 (td, J=7.6, 1.5 Hz, 1H), 6.96 (dd, J=7.7, 1.4 Hz, 1H), 6.89 (dd, J=7.9, 1.1 Hz, 1H), 6.82 (td, J=7.4, 1.2 Hz, 1H), 6.32 (d, J=1.6 Hz, 1H), 6.25 (d, J=1.8 Hz, 1H), 6.07 (t, J=5.6 Hz, 1H), 4.39 (d, J=5.3 Hz, 2H), 3.28-3.06 (m, 8H), 1.40 (s, 9H).

¹³C NMR (101 MHz, DMSO) δ 168.89, 153.73, 142.02, 139.23, 134.14, 129.61, 129.01, 128.44, 127.54, 127.11, 126.81, 126.19, 122.45, 117.17, 116.22, 115.24, 114.24, 109.23, 79.13, 46.38, 28.04.

MS (ESI) m/z=517.2 [M+H]⁺, 561.3 [M+HCOO]⁻.

### tert-Butyl 4-(1-((4-fluorobenzyl)amino)-10H-phenothiazine-3-carbonyl)piperazine-1-carboxylate

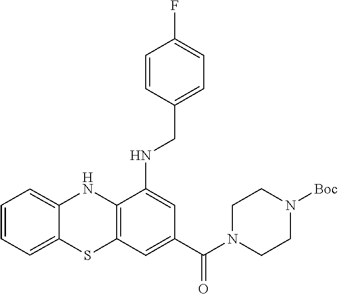 [SMILES: CC(C)(C)OC(=O)N1CCN(C(=O)c2cc(NCc3ccc(F)cc3)c3c(c2)Sc2ccccc2N3)CC1]

General procedure D was followed using compound 19 (0.122 g, 0.286 mmol), acetic acid (0.38 ml, 6.58 mmol), 4-fluorobenzaldehyde as corresponding aldehyde (0.092 ml, 0.858 mmol) and sodium cyanoborohydride (0.045 g, 0.715 mmol). The reaction mixture was stirred overnight and purified by reversed-phase column chromatography (10-80% MeOH in water). This resulted in 0.122 g of compound 41 (80%).

¹H NMR (400 MHz, DMSO) δ 7.94 (s, 1H), 7.42-7.33 (m, 2H), 7.22-7.11 (m, 2H), 7.04 (td, J=7.6, 1.5 Hz, 1H), 6.96 (dd, J=7.7, 1.4 Hz, 1H), 6.92-6.86 (m, 1H), 6.81 (td, J=7.4, 1.3 Hz, 1H), 6.33 (d, J=1.6 Hz, 1H), 6.24 (s, 1H), 6.06 (t, J=5.6 Hz, 1H), 4.38 (d, J=5.3 Hz, 2H), 4.12 (q, J=5.3 Hz, 1H), 3.36 (s, 6H), 3.17 (d, J=4.9 Hz, 1H), 1.40 (s, 9H).

¹³C NMR (101 MHz, DMSO) δ 168.92, 161.68 (d, J=242.5 Hz), 153.73, 141.99, 135.73 (d, J=2.9 Hz), 134.00, 129.68, 129.05, 128.96, 127.54, 126.18, 122.46, 117.17, 116.29, 115.25, 115.04, 114.33, 109.23, 79.15, 48.61, 45.64, 28.00.

MS (ESI) m/z=579.3 [M+HCOO]⁻.

### tert-Butyl 4-(2-(1-(benzylamino)-10H-phenothiazine-3-carboxamido)ethyl)piperazine-1-carboxylate (42)

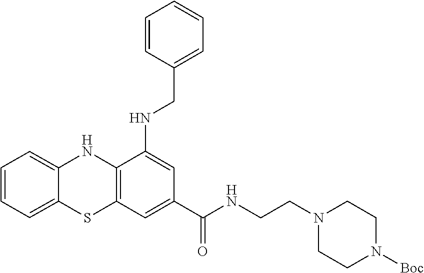 [SMILES: CC(C)(C)OC(=O)N1CCN(CCNC(=O)c2cc(NCc3ccccc3)c3c(c2)Sc2ccccc2N3)CC1]

General procedure D was followed using compound 20 (0.10 g, 0.213 mmol), acetic acid (0.280 ml, 4.90 mmol), benzaldehyde as corresponding aldehyde (0.065 ml, 0.639 mmol) and sodium cyanoborohydride (0.033 g, 0.532 mmol). The reaction mixture was stirred overnight and purified by reversed-phase column chromatography (10-80% MeOH in water). This resulted in 0.068 g of compound 42 (57%).

¹H NMR (400 MHz, DMSO) δ 8.08 (t, J=5.7 Hz, 1H), 7.97 (s, 1H), 7.42-7.31 (m, 4H), 7.30-7.21 (m, 1H), 7.02 (td, J=7.6, 1.5 Hz, 1H), 6.95 (dd, J=7.7, 1.4 Hz, 1H), 6.87 (dd, J=8.8, 1.5 Hz, 2H), 6.81 (td, J=6.6, 2.6 Hz, 2H), 5.88 (t, J=5.6 Hz, 1H), 4.38 (d, J=5.3 Hz, 2H), 3.27 (q, J=6.4 Hz, 6H), 2.41 (d, J=7.0 Hz, 1H), 2.33 (t, J=5.0 Hz, 4H), 1.39 (s, 9H).

¹³C NMR (101 MHz, DMSO) δ 165.55, 153.57, 141.74, 139.31, 134.49, 130.68, 128.41, 128.26, 127.43, 127.08, 126.31, 122.47, 117.11, 115.51, 115.25, 113.97, 109.23, 78.71, 56.94, 54.91, 52.46, 46.83, 36.61, 28.05.

MS (ESI) m/z=560.3 [M+H]⁺, 604.41 [M+HCOO]⁻.

### tert-Butyl 4-(2-(1-((4-fluorobenzyl)amino)-10H-phenothiazine-3-carboxamido)ethyl)piperazine-1-carboxylate (43)

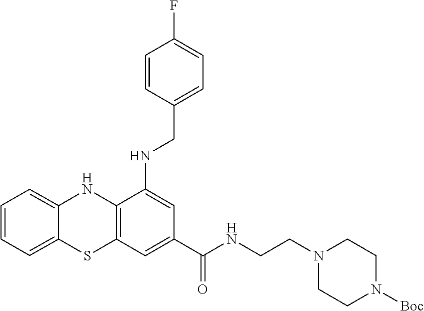 [SMILES: CC(C)(C)OC(=O)N1CCN(CCNC(=O)c2cc(NCc3ccc(F)cc3)c3c(c2)Sc2ccccc2N3)CC1]

General procedure D was followed using compound 20 (0.1 g, 0.213 mmol), acetic acid (0.280 ml, 4.90 mmol), 4-fluorobenzaldehyde as corresponding aldehyde (0.069 ml, 0.639 mmol) and sodium cyanoborohydride (0.033 g, 0.532 mmol). The reaction mixture was stirred overnight and purified by reversed-phase column chromatography (10-80% MeOH in water). This resulted in 0.034 g of compound 43 (28%).

¹H NMR (400 MHz, DMSO) δ 8.08 (t, J=5.7 Hz, 1H), 7.96 (s, 1H), 7.42 (dd, J=8.5, 5.6 Hz, 2H), 7.18 (t, J=8.8 Hz, 2H), 7.06-6.98 (m, 1H), 6.98-6.92 (m, 1H), 6.90-6.76 (m, 4H), 5.88 (t, J=5.4 Hz, 1H), 4.36 (d, J=5.3 Hz, 2H), 3.30-3.24 (m, 6H), 2.40 (t, J=6.9 Hz, 2H), 2.33 (t, J=5.0 Hz, 4H), 1.39 (s, 9H).

¹³C NMR (101 MHz, DMSO) δ 165.46, 161.26 (d, J=242.6 Hz), 153.81, 141.74, 135.45 (d, J=2.9 Hz), 134.36, 130.78, 129.32 (d, J=8.0 Hz), 128.19, 127.47, 126.11, 122.47, 117.09, 115.58, 115.29, 115.13 (d, J=21.3 Hz), 114.06, 109.25, 78.71, 56.95, 52.47, 46.06, 36.62, 28.06.

MS (ESI) m/z=578.3 [M+H]⁺, 622.4 [M+HCOO]⁻.

### tert-Butyl 4-(2-(1-(cyclohexylamino)-10H-phenothiazine-3-carboxamido)ethyl)piperazine-1-carboxylate (44)

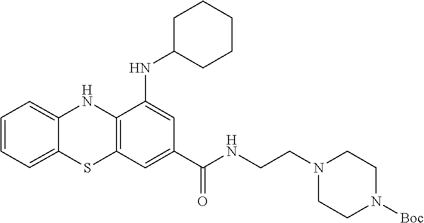 [SMILES: CC(C)(C)OC(=O)N1CCN(CCNC(=O)c2cc(NC3CCCCC3)c3c(c2)Sc2ccccc2N3)CC1]

General procedure D was followed using compound 20 (0.50 g, 0.106 mmol), acetic acid (0.140 ml, 2.449 mmol), cyclohexanone as corresponding ketone (0.033 ml, 0.319 mmol) and sodium cyanoborohydride (0.017 g, 0.266 mmol). The reaction mixture was stirred overnight and purified by reversed-phase column chromatography (10-80% MeOH in water) followed by preparative TLC. This resulted in 0.030 g of compound 44 (51%).

¹H NMR (400 MHz, DMSO) δ 8.12 (t, J=5.6 Hz, 1H), 7.91 (s, 1H), 7.05-6.99 (m, 1H), 6.94 (dd, J=7.7, 1.4 Hz, 1H), 6.90-6.86 (m, 2H), 6.82-6.76 (m, 2H), 4.99 (d, J=7.2 Hz, 1H), 3.30 (q, J=5.8 Hz, 6H), 2.44 (t, J=6.9 Hz, 2H), 2.36 (t, J=5.0 Hz, 4H), 2.00 (d, J=12.0 Hz, 2H), 1.76 (d, J=13.0 Hz, 2H), 1.65 (d, J=12.5 Hz, 1H), 1.39 (s, 9H), 1.42-1.30 (m, 2H), 1.21 (dd, J=14.4, 8.8 Hz, 4H).

¹³C NMR (101 MHz, DMSO) δ 165.65, 153.83, 141.86, 133.70, 130.59, 128.35, 127.42, 126.11, 122.38, 117.26, 115.86, 115.22, 113.45, 109.33, 78.71, 56.90, 52.48, 51.32, 36.68, 32.79, 28.05, 25.61, 24.87.

MS (ESI) m/z=552.1 [M+H]⁺.

### 1-(Benzylamino)-N-(2-(methylamino)ethyl)-10H-phenothiazine-3-carboxamide trihydrochloride (45, CPD-017)

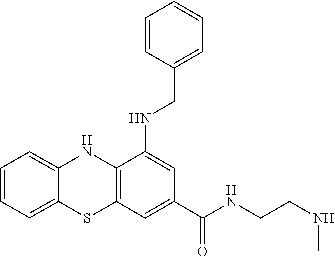 [SMILES: CNCCNC(=O)c1cc(NCc2ccccc2)c2c(c1)Sc1ccccc1N2]

General procedure E was followed using compound 31 (0.089 g, 0.176 mmol) and 4M HCl in dioxane (0.66 ml, 2.65 mmol) to afford 0.076 g of the desired compound 45 (84%). The reported NMR spectrum refers to trihydrochloride salt.

¹H NMR (400 MHz, DMSO) δ 8.94 (d, J=8.7 Hz, 2H), 8.54 (t, J=5.7 Hz, 2H), 7.41 (d, J=7.5 Hz, 2H), 7.32 (t, J=7.4 Hz, 2H), 7.23 (t, J=7.3 Hz, 1H), 7.12-6.74 (m, 6H), 4.41 (s, 2H), 3.46 (d, J=6.6 Hz, 2H), 3.00 (p, J=5.9 Hz, 2H), 2.53 (t, J=5.3 Hz, 3H).

¹³C NMR (101 MHz, DMSO) δ 166.09, 141.72, 138.93, 133.46, 131.68, 128.36, 127.69, 127.40, 127.00, 126.02, 122.53, 116.91, 115.72, 115.49, 114.98, 110.46, 48.62, 48.01, 47.04, 35.68, 32.48.

MS (ESI) m/z=405.2 [M+H]⁺, 449.3 [M+HCOO]⁻.

HRMS: calc 405, 1744, found 405, 1728 [M+H]⁺.

### 1-((4-Fluorobenzyl)amino)-N-(2-(methylamino)ethyl)-10H-phenothiazine-3-carboxamide trihydrochloride (46, CPD-018)

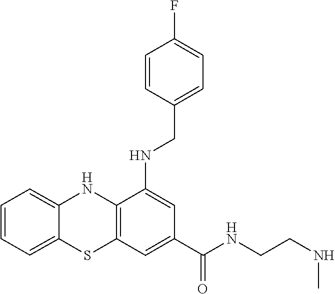 [SMILES: CNCCNC(=O)c1cc(NCc2ccc(F)cc2)c2c(c1)Sc1ccccc1N2]

General procedure E was followed using compound 32 (0.107 g, 0,205 mmol) and 4M HCl in dioxane (0.77 ml, 3.07 mmol) to afford 0.100 g of the desired compound 46 (92%). The reported NMR spectrum refers to trihydrochloride salt.

¹H NMR (400 MHz, DMSO) δ 8.92 (d, J=8.4 Hz, 2H), 8.55 (t, J=5.9 Hz, 2H), 7.45 (dd, J=8.4, 5.5 Hz, 2H), 7.14 (t, J=8.7 Hz, 2H), 7.09-6.75 (m, 6H), 4.40 (s, 2H), 3.47 (q, J=5.8 Hz, 2H), 3.01 (p, J=5.7 Hz, 2H), 2.53 (t, J=5.4 Hz, 3H).

¹³C NMR (101 MHz, DMSO) δ 166.06, 161.32 (d, J=242.3 Hz), 141.68, 134.97, 133.12, 131.83, 129.67 (d, J=8.1 Hz), 127.41, 126.03, 122.57, 116.89, 115.82, 115.49, 115.08 (d, J=21.3 Hz), 110.67, 48.62, 48.02, 46.36, 35.68, 32.48, 31.34.

MS (ESI) m/z=423.2 [M+H]⁺, 467.2 [M+HCOO]⁻.

HRMS: calc 423, 1649, found 423, 1649 [M+H]⁺.

### 1-(Cyclohexylamino)-N-(2-(methylamino)ethyl)-10H-phenothiazine-3-carboxamide trihydrochloride (47, CPD-019)

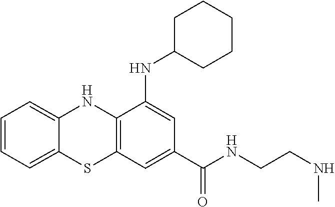 [SMILES: CNCCNC(=O)c1cc(NC2CCCCC2)c2c(c1)Sc1ccccc1N2]

General procedure E was followed using compound 33 (0.051 g, 0.103 mmol) and 4M HCl in dioxane (0.385 ml, 1.540 mmol) to afford 0.050 g of the desired compound 47 (96%). The reported NMR spectrum refers to trihydrochloride salt.

¹H NMR (400 MHz, DMSO) δ 8.83 (s, 2H), 8.65 (d, J=5.7 Hz, 1H), 7.33-7.16 (m, 2H), 7.11-6.92 (m, 3H), 6.84 (td, J=7.4, 1.6 Hz, 1H), 3.51 (q, J=5.9 Hz, 2H), 3.36 (td, J=9.3, 4.0 Hz, 1H), 3.05 (p, J=6.0 Hz, 2H), 2.56 (t, J=5.4 Hz, 3H), 2.02-1.94 (m, 2H), 1.75 (d, J=13.1 Hz, 2H), 1.62 (d, J=12.7 Hz, 1H), 1.53-1.08 (m, 5H).

MS (ESI) m/z=397.1 [M+H]⁺.

### 1-(Cyclopentylamino)-N-(2-(methylamino)ethyl)-10H-phenothiazine-3-carboxamide trihydrochloride (48, CPD-020)

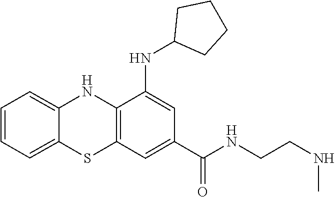 [SMILES: CNCCNC(=O)c1cc(NC2CCCC2)c2c(c1)Sc1ccccc1N2]

General procedure E was followed using compound 34 (0.069 g, 0.143 mmol) and 4M HCl in dioxane (0.536 ml, 2.144 mmol) to afford 0.064 g of the desired compound 48 (90%). The reported NMR spectrum refers to trihydrochloride salt.

¹H NMR (400 MHz, DMSO) δ 9.03 (s, 1H), 8.94-8.88 (m, 2H), 8.67 (s, 1H), 7.37-7.20 (m, 2H), 7.16-6.92 (m, 2H), 6.84 (s, 1H), 3.86 (s, 1H), 3.51 (s, 2H), 3.10-2.99 (m, 2H), 2.59-2.52 (m, 3H), 1.99-1.88 (m, 2H), 1.80-1.66 (m, 5H), 1.58-1.51 (m, 2H).

MS (ESI) m/z=383.3 [M+H]⁺.

### N-(2-aminoethyl)-1-(benzylamino)-10H-phenothiazine-3-carboxamide trihydrochloride (49, CPD-021)

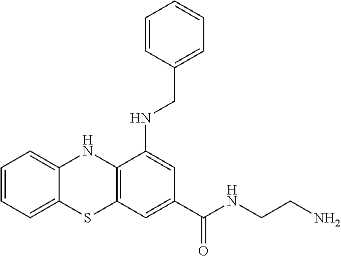 [SMILES: NCCNC(=O)c1cc(NCc2ccccc2)c2c(c1)Sc1ccccc1N2]

General procedure E was followed using compound 35 (0.122 g, 0.249 mmol) and 4M HCl in dioxane (0.932 ml, 3.73 mmol) to afford 0.056 g of the desired 49 (45%). The reported NMR spectrum refers to trihydrochloride salt.

¹H NMR (400 MHz, DMSO) δ 8.60-8.49 (m, 2H), 8.11 (s, 3H), 7.41 (d, J=7.5 Hz, 2H), 7.31 (t, J=7.4 Hz, 2H), 7.22 (t, J=7.3 Hz, 1H), 7.14-6.72 (m, 6H), 4.41 (s, 2H), 3.43 (q, J=6.0 Hz, 2H), 2.91 (h, J=5.8 Hz, 2H).

¹³C NMR (101 MHz, DMSO) δ 166.04, 141.65, 138.51, 132.02, 129.55, 129.46, 129.22, 128.36, 127.87, 127.45, 127.11, 126.05, 122.61, 116.89, 115.93, 115.54, 111.20, 47.41, 38.67, 37.09.

MS (ESI) m/z=391.2 [M+H]⁺, 389.3 [M−H]⁻.

HRMS: calc 391.1587, found 391.1586 [M+H]⁺.

### N-(2-aminoethyl)-1-((4-fluorobenzyl)amino)-10H-phenothiazine-3-carboxamide trihydrochloride (50, CPD-022)

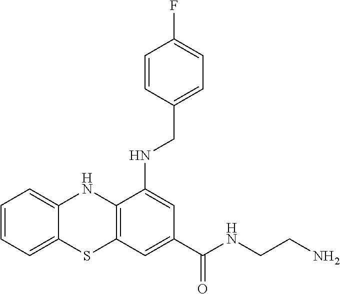 [SMILES: NCCNC(=O)c1cc(NCc2ccc(F)cc2)c2c(c1)Sc1ccccc1N2]

General procedure E was followed using compound 36 (0.030 g, 0.059 mmol) and 4M HCl in dioxane (0.221 ml, 0.885 mmol) to afford 0.015 g of the desired compound 50 (50%). The reported NMR spectrum refers to trihydrochloride salt.

¹H NMR (400 MHz, DMSO) δ 8.61-8.46 (m, 1H), 8.11 (s, 3H), 7.45 (dd, J=8.4, 5.5 Hz, 2H), 7.20-6.72 (m, 7H), 4.39 (s, 2H), 3.43 (p, J=6.1 Hz, 2H), 2.92 (p, J=5.9 Hz, 2H).

¹³C NMR (101 MHz, DMSO) δ 166.02, 162.53, 160.12, 141.68, 134.93, 133.02, 131.84, 129.75, 129.67, 127.44, 126.01, 122.53, 116.85, 115.51, 115.17, 114.96, 110.71, 48.62, 46.37, 37.07.

MS (ESI) m/z 409.2 [M+H]⁺, m/z 453.3 [M+HCOO]⁻.

HRMS: calc 409.1493, found 409.1490 [M+H]⁺.

### N-(2-aminoethyl)-1-((2,4-difluorobenzyl)amino)-10H-phenothiazine-3-carboxamide trihydrochloride (51, CPD-023)

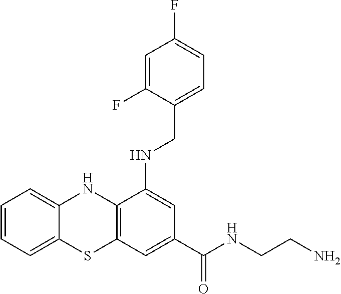 [SMILES: NCCNC(=O)c1cc(NCc2ccc(F)cc2F)c2c(c1)Sc1ccccc1N2]

General procedure E was followed using compound 37 (0.075 g, 0.142 mmol) and 4M HCl in dioxane (0.534 ml, 2.136 mmol) to afford 0.048 g of the desired compound 51 (63%). The reported NMR spectrum refers to trihydrochloride salt.

¹H NMR (400 MHz, DMSO) δ 8.52-8.39 (m, 2H), 8.05 (s, 3H), 7.52-7.21 (m, 11H), 7.20-6.85 (m, 7H), 6.79 (t, J=7.4 Hz, 1H), 4.48-4.36 (m, 2H), 3.49-3.37 (m, 2H), 2.91 (p, J=5.8 Hz, 2H).

¹³C NMR (101 MHz, DMSO) δ 166.13, 141.81, 133.90, 131.40, 130.98, 127.44, 126.03, 122.53, 116.95, 115.69, 114.78, 111.56, 111.35, 110.44, 110.19, 109.56, 103.79, 102.28, 45.76, 38.69, 37.07.

MS (ESI) m/z=427.2 [M+H]⁺, 471.2 [M+HCOO]⁻.

HRMS: calc 427.1399, found 427.1391 [M+H]⁺.

### N-(2-aminoethyl)-1-((3,5-difluorobenzyl)amino)-10H-phenothiazine-3-carboxamide trihydrochloride (52, CPD-024)

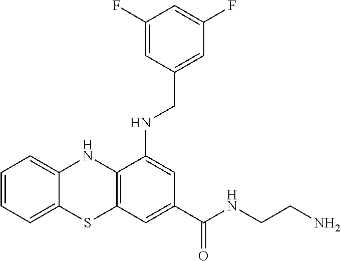 [SMILES: NCCNC(=O)c1cc(NCc2cc(F)cc(F)c2)c2c(c1)Sc1ccccc1N2]

General procedure E was followed using compound 38 (0.065 g, 0.123 mmol) and 4M HCl in dioxane (0.463 ml, 1.851 mmol) to afford 0.030 g of the desired compound 52 (45%). The reported NMR spectrum refers to trihydrochloride salt.

¹H NMR (400 MHz, DMSO) δ 8.56-8.36 (m, 2H), 8.06 (d, J=6.8 Hz, 3H), 7.47 (q, J=8.3 Hz, 1H), 7.25 (td, J=9.9, 2.4 Hz, 1H), 7.14 (t, J=7.0 Hz, 1H), 7.09-6.97 (m, 2H), 6.96-6.75 (m, 4H), 4.50-4.35 (m, 2H), 3.42 (q, J=5.8 Hz, 2H), 2.91 (h, J=5.4 Hz, 2H).

¹³C NMR (101 MHz, DMSO) δ 166.15, 161.65 (d, J=12.4 Hz), 160.29 (d, J=12.2 Hz), 159.19 (d, J=12.5 Hz), 141.79, 133.93, 131.36, 130.96, 127.44, 126.06, 122.54, 116.96, 115.09 (d, J=78.9 Hz), 111.47 (d, J=21.0 Hz), 110.31 (d, J=24.9 Hz), 109.43, 103.80 (t, J=25.8 Hz), 48.63, 38.69, 37.08.

MS (ESI) m/z=427.2 [M+H]⁺, 471.2 [M+HCOO]⁻.

HRMS: calc 427.1399, found 427.1393 [M+H]⁺.

### N-(2-aminoethyl)-1-((4-(trifluoromethyl)benzyl)amino)-10H-phenothiazine-3-carboxamide trihydrochloride (53, CPD-025)

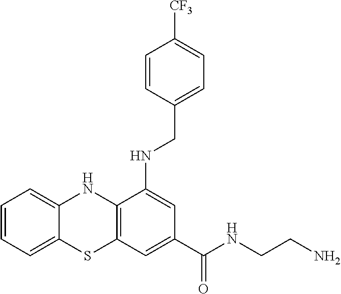 [SMILES: NCCNC(=O)c1cc(NCc2ccc(C(F)(F)F)cc2)c2c(c1)Sc1ccccc1N2]

General procedure E was followed using compound 39 (0.064 g, 0.115 mmol) and 4M HCl in dioxane (0.430 ml, 1.719 mmol) to afford 0.055 g of the desired 53 (85%). The reported NMR spectrum refers to trihydrochloride salt.

¹H NMR (400 MHz, DMSO) δ 8.51 (dd, J=15.5, 10.0 Hz, 2H), 8.08 (s, 3H), 7.76-7.52 (m, 4H), 7.12 (d, J=8.0 Hz, 1H), 7.04-6.73 (m, 5H), 4.51 (s, 2H), 3.40 (t, J=5.8 Hz, 2H), 2.90 (q, J=6.1 Hz, 2H).

MS (ESI) m/z=459.2 [M+H]1, 503.2 [M+HCOO]⁻.

HRMS: calc 459.1461, found 459.1457 [M+H]⁺.

### (1-(Benzylamino)-10H-phenothiazin-3-yl)(piperazin-1-yl)methanone trihydrochloride (54, CPD-026)

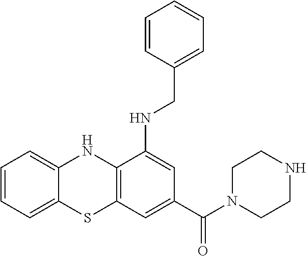 [SMILES: O=C(c1cc(NCc2ccccc2)c2c(c1)Sc1ccccc1N2)N1CCNCC1]

General procedure E was followed using compound 40 (0.066 g, 0.128 mmol) and 4M HCl in dioxane (0.479 ml, 1.916 mmol) to afford 0.059 g of the desired compound 54 (88%). The reported NMR spectrum refers to trihydrochloride salt.

¹H NMR (400 MHz, DMSO) δ 9.50-9.45 (m, 2H), 8.57-8.52 (m, 1H), 7.54-6.74 (m, 6H), 6.43-6.38 (m, 1H), 5.95-5.87 (m, 4H), 4.40-4.36 (m, 2H), 3.54-3.50 (m, 4H), 2.94-2.85 (m, 4H).

¹³C NMR (101 MHz, DMSO) δ 169.03, 142.06, 138.71, 133.23, 130.62, 128.43, 128.12, 127.51, 127.02, 126.06, 122.49, 118.63, 116.95, 116.44, 115.51, 115.02, 110.17, 48.63, 46.74, 42.36.

MS (ESI) m/z=417.3 [M+H]⁺, 461.3 [M+HCOO]⁻.

HRMS: calc 417.1744, found 417.1737 [M+H]⁺.

### (1-((4-Fluorobenzyl)amino)-10H-phenothiazin-3-yl)(piperazin-1-yl)methanone trihydrochloride (55, CPD-027)

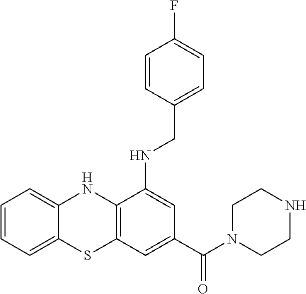 [SMILES: O=C(c1cc(NCc2ccc(F)cc2)c2c(c1)Sc1ccccc1N2)N1CCNCC1]

General procedure E was followed using compound 41 (0.059 g, 0.110 mmol) and 4M HCl in dioxane (0.414 ml, 1.655 mmol) to afford 0.53 g of the desired compound 55 (88%). The reported NMR spectrum refers to trihydrochloride salt.

¹H NMR (400 MHz, DMSO) δ 9.39-9.34 (m, 2H), 8.45-8.40 (m, 1H), 7.41 (t, J=6.9 Hz, 2H), 7.30-6.69 (m, 5H), 6.49-6.26 (m, 2H), 4.38-4.33 (m, 2H), 3.57-3.53 (m, 4H), 3.01-2.96 (m, 4H).

¹³C NMR (101 MHz, DMSO) δ 169.03, 161.21 (d, J=242.3 Hz), 142.06, 135.16, 133.79, 130.29, 129.31, 129.23, 128.19, 127.43, 126.05, 122.41, 116.97, 116.28, 115.41, 115.22, 115.01, 114.49, 109.47, 45.74, 42.38.

MS (ESI) m/z=435.2 [M+H]⁺, 479.2 [M+HCOO]⁻.

HRMS: calc 435.1649, found 435.1644 [M+H]⁺.

### 1-(Benzylamino)-N-(2-(piperazin-1-yl)ethyl)-10H-phenothiazine-3-carboxamide tetrahydrochloride (56, CPD-028)

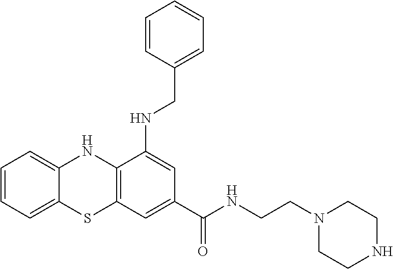 [SMILES: O=C(NCCN1CCNCC1)c1cc(NCc2ccccc2)c2c(c1)Sc1ccccc1N2]

General procedure E was followed using compound 42 (0.091 g, 0.194 mmol) and 4M HCl in dioxane (0.727 ml, 1.916 mmol) to afford 0.077 g of the desired compound 56 (83%). The reported NMR spectrum refers to tetrahydrochloride salt.

¹H NMR (400 MHz, DMSO) δ 11.69-11.64 (m, 1H), 9.92-9.87 (m, 2H), 8.68-8.41 (m, 2H), 7.51-7.19 (m, 5H), 7.19-6.75 (m, 5H), 4.43-4.38 (m, 21H), 3.89-3.22 (m, 12H).

¹³C NMR (101 MHz, DMSO) δ 166.35, 142.22, 139.72, 134.47, 131.87, 128.79, 128.00, 127.84, 127.65, 127.35, 126.46, 122.93, 117.40, 116.08, 115.90, 114.97, 110.25, 55.80, 48.43, 47.22, 34.21.

MS (ESI) m/z=460.2 [M+H]⁺, 504.3 [M+HCOO]⁻.

### 1-((4-Fluorobenzyl)amino)-N-(2-(piperazin-1-yl)ethyl)-10H-phenothiazine-3-carboxamide tetrahydrochloride (57, CPD-029)

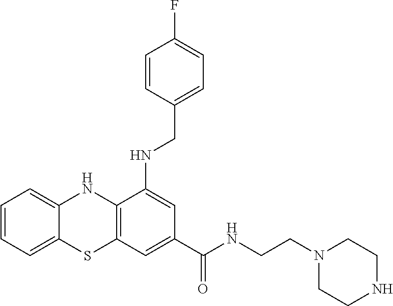 [SMILES: O=C(NCCN1CCNCC1)c1cc(NCc2ccc(F)cc2)c2c(c1)Sc1ccccc1N2]

General procedure E was followed using compound 57 (0.034 g, 0.059 mmol) and 4M HCl in dioxane (0.221 ml, 0.883 mmol) to afford 0.030 g of the desired compound 43 (88%). The reported NMR spectrum refers to tetrahydrochloride salt.

¹H NMR (400 MHz, DMSO) δ 11.55 (s, 1H), 9.75-9.71 (m, 2H), 8.59-8.54 (m, 1H), 8.36-8.31 (m, 1H), 7.45 (t, J=6.8 Hz, 2H), 7.16 (t, J=8.7 Hz, 2H), 7.02-7.02 (m, 1H), 6.96-6.88 (m, 3H), 6.80 (t, J=7.2 Hz, 1H), 4.39 (s, 2H), 4.31-3.15 (m, 12H).

¹³C NMR (101 MHz, DMSO) δ 165.92, 161.24 (d, J=242.1 Hz), 141.71, 135.49, 134.18, 131.36, 129.38 (d, J=7.8 Hz), 127.41, 127.19, 126.03, 122.49, 116.94, 115.61, 115.41, 115.08 (d, J=21.1 Hz), 114.42, 109.50, 55.35, 48.01, 45.93, 33.76.

MS (ESI) m/z=478.3 [M+H]⁺, 522.3 [M+HCOO]⁻.

### 1-(Cyclohexylamino)-N-(2-(piperazin-1-yl)ethyl)-10H-phenothiazine-3-carboxamide tetrahydrochloride (58, CPD-030)

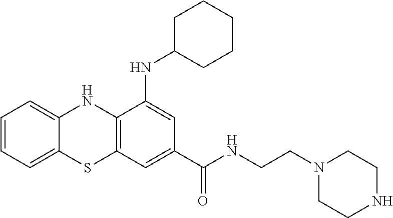 [SMILES: O=C(NCCN1CCNCC1)c1cc(NC2CCCCC2)c2c(c1)Sc1ccccc1N2]

General procedure E was followed using compound 44 (0.030 g, 0.054 mmol) and 4M HCl in dioxane (0.204 ml, 0.816 mmol) to afford 0.020 g of the desired compound 58 (83%). The reported NMR spectrum refers to tetrahydrochloride salt (purity 91%).

¹H NMR (400 MHz, DMSO) δ 9.77 (s, 2H), 8.69 (s, 1H), 7.39-7.15 (m, 2H), 7.14-6.88 (m, 3H), 6.80 (t, J=7.4 Hz, 1H), 3.61 (d, J=6.0 Hz, 2H), 3.42-3.27 (m, 4H), 3.13 (s, 1H), 2.47 (p, J=1.8 Hz, 5H), 1.95 (d, J=11.7 Hz, 2H), 1.71 (d, J=9.5 Hz, 2H), 1.58 (d, J=12.4 Hz, 1H), 1.41 (d, J=12.2 Hz, 1H), 1.34-1.06 (m, 3H).

MS (ESI) m/z=452.4 [M+H]⁺.

### Morpholino(10H-phenothiazin-3-yl)methanone (60, CPD-031)

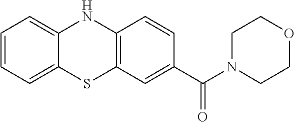 [SMILES: O=C(c1ccc2c(c1)Sc1ccccc1N2)N1CCOCC1]

General procedure B was followed using compound 59 (0.040 g, 0.164 mmol), morpholine as corresponding amine (0.035 ml, 0.411 mmol), HATU (0.088 g, 0.230 mmol) and DIPEA (0.086 ml, 0.493 mmol). The reaction mixture was stirred for 16 hours and purified by reversed-phase column chromatography (10-80% MeOH in water) to give 0.048 g (93%) of compound 60.

¹H NMR (400 MHz, DMSO) δ 8.93 (s, 1H), 7.08-6.88 (m, 4H), 6.77 (td, J=7.5, 1.3 Hz, 1H), 6.69 (dd, J=8.1, 1.9 Hz, 2H), 3.57 (t, J=4.6 Hz, 4H), 3.49-3.44 (m, 2H), 2.94-2.85 (m, 2H).

¹³C NMR (101 MHz, DMSO) δ 168.37, 143.41, 141.24, 128.47, 127.76, 127.27, 126.29, 125.57, 122.28, 116.27, 115.92, 114.68, 113.70, 66.10.

MS (ESI) m/z=313.1 [M+H]⁺.

### N-(2-morpholinoethyl)-10H-phenothiazine-3-carboxamide (61, CPD-032)

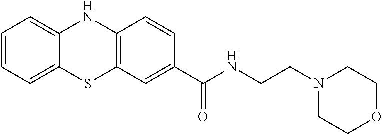 [SMILES: O=C(NCCN1CCOCC1)c1ccc2c(c1)Sc1ccccc1N2]

General procedure B was followed using compound 59 (0.040 g, 0.164 mmol), 2-morpholinoethan-1-amine as corresponding amine (0.054 g, 0.411 mmol), HATU (0.088 g, 0.230 mmol) and DIPEA (0.086 ml, 0.493 mmol). The reaction mixture was stirred for 16 hours and purified by reversed-phase column chromatography (10-80% MeOH in water) to give 0.042 g (72%) of compound 61.

¹H NMR (400 MHz, DMSO) δ 8.94 (s, 1H), 8.18 (t, J=5.7 Hz, 1H), 7.47 (dd, J=8.3, 2.0 Hz, 1H), 7.39 (d, J=1.9 Hz, 1H), 6.99 (td, J=7.6, 1.5 Hz, 1H), 6.91 (dd, J=7.6, 1.5 Hz, 1H), 6.77 (td, J=7.5, 1.3 Hz, 1H), 6.72-6.64 (m, 2H), 3.55 (t, J=4.6 Hz, 4H), 3.32 (q, J=6.6 Hz, 2H), 2.45-2.35 (m, 6H).

¹³C NMR (101 MHz, DMSO) δ 165.01, 144.39, 140.94, 127.78, 127.71, 127.28, 126.32, 125.18, 122.41, 115.92, 115.85, 114.76, 113.61, 66.23, 57.49, 53.35, 36.49.

MS (ESI) m/z=356.2 [M+H]⁺.

### tert-Butyl (2-(10H-phenothiazine-3-carboxamido)ethyl)(methyl)carbamate (62)

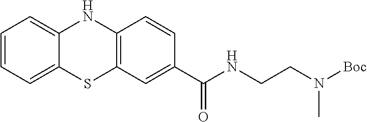 [SMILES: CN(CCNC(=O)c1ccc2c(c1)Sc1ccccc1N2)C(=O)OC(C)(C)C]

General procedure B was followed using compound 59 (0.060 g, 0.247 mmol), 1-boc-1-methyl-ethylenediamine as corresponding amine (0.107 g, 0.617 mmol), HATU (0.131 g, 0.345 mmol) and DIPEA (0.129 ml, 0.740 mmol). The reaction mixture was stirred for 16 hours and purified by reversed-phase column chromatography (10-80% MeOH in water) to give 0.081 g (82%) of compound 62.

¹H NMR (400 MHz, DMSO) δ 8.89 (s, 1H), 8.37-8.20 (m, 1H), 7.53-7.31 (m, 2H), 6.99 (td, J=7.6, 1.5 Hz, 1H), 6.91 (dd, J=7.7, 1.5 Hz, 1H), 6.77 (td, J=7.5, 1.2 Hz, 1H), 6.71-6.62 (m, 2H). 3.30 (d. J=2.7 Hz. 4H). 2.79 (d. J=10.3 Hz. 3H). 1.32 (d. 9H).

¹³C NMR (101 MHz, DMSO) δ 165.02, 156.28, 144.30, 140.87, 127.74, 127.33, 127.29, 126.28, 125.12, 122.38, 115.88, 115.72, 114.71, 113.52, 77.91, 30.73, 27.98.

MS (ESI) m/z=300.1 [M−Boc+H]⁺.

### tert-Butyl (2-(10H-phenothiazine-3-carboxamido)ethyl)carbamate (63)

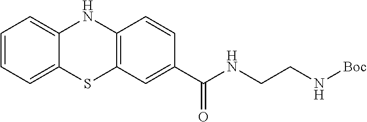 [SMILES: CC(C)(C)OC(=O)NCCNC(=O)c1ccc2c(c1)Sc1ccccc1N2]

General procedure B was followed using compound 59 (0.060 g, 0.247 mmol), tert-butyl (2-aminoethyl)carbamate as corresponding amine (0.099 g, 0.617 mmol), HATU (0.131 g, 0.345 mmol) and DIPEA (0.129 ml, 0.740 mmol). The reaction mixture was stirred for 16 hours and purified by reversed-phase column chromatography (10-80% MeOH in water) to give 0.063 g (66%) of compound 63.

¹H NMR (400 MHz, DMSO) δ 8.89 (s, 1H), 8.20 (t, J=5.6 Hz, 1H), 7.46 (dd, J=8.3, 2.0 Hz, 1H), 7.38 (d, J=2.0 Hz, 1H), 6.99 (td, J=7.6, 1.5 Hz, 1H), 6.95-6.86 (m, 2H), 6.77 (td, J=7.5, 1.3 Hz, 1H), 6.71-6.62 (m, 2H), 3.22 (q, J=6.2 Hz, 2H), 3.05 (q, J=6.3 Hz, 2H), 1.37 (s, 9H).

¹³C NMR (101 MHz, DMSO) δ 165.19, 155.72, 144.30, 140.87, 127.73, 127.69, 127.31, 126.27, 125.18, 122.37, 115.90, 115.74, 114.70, 113.51, 77.67, 28.24.

MS (ESI) m/z=286.1 [M−Boc+H]⁺, 430.2 [M+HOO]⁻.

### tert-Butyl 4-(10H-phenothiazine-3-carbonyl)piperazine-1-carboxylate (64)

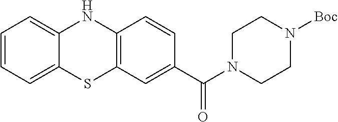 [SMILES: CC(C)(C)OC(=O)N1CCN(C(=O)c2ccc3c(c2)Sc2ccccc2N3)CC1]

General procedure B was followed using compound 59 (0.060 g, 0.247 mmol), tert-butyl piperazine-1-carboxylate as corresponding amine (0.115 g, 0.617 mmol), HATU (0.131 g, 0.345 mmol) and DIPEA (0.129 ml, 0.740 mmol). The reaction mixture was stirred for 16 hours and purified by reversed-phase column chromatography (10-80% MeOH in water) to give 0.034 g (34%) of compound 64.

¹H NMR (400 MHz, DMSO) δ 8.87 (s, 1H), 7.05 (dd, J=8.1, 1.9 Hz, 1H), 7.00 (td, J=7.6, 1.5 Hz, 1H), 6.97 (d, J=1.9 Hz, 1H), 6.92 (dd, J=7.7, 1.4 Hz, 1H), 6.78 (td, J=7.5, 1.3 Hz, 1H), 6.73-6.64 (m, 2H), 3.45-3.29 (m, 8H), 1.40 (s, 9H).

¹³C NMR (101 MHz, DMSO) δ 168.47, 153.81, 143.40, 141.20, 128.62, 127.75, 127.26, 126.30, 125.56, 122.30, 116.28, 115.93, 114.64, 113.66, 79.16, 28.03.

MS (ESI) m/z=412.3 [M+H]⁺.

### tert-Butyl 4-(2-(10H-phenothiazine-3-carboxamido)ethyl)piperazine-1-carboxylate (65)

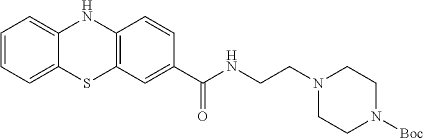 [SMILES: CC(C)(C)OC(=O)N1CCN(CCNC(=O)c2ccc3c(c2)Sc2ccccc2N3)CC1]

General procedure B was followed using compound 59 (0.060 g, 0.247 mmol), tert-butyl 4-(2-aminoethyl)piperazine-1-carboxylate as corresponding amine (0.141 g, 0.617 mmol), HATU (0.131 g, 0.345 mmol) and DIPEA (0.129 ml, 0.740 mmol). The reaction mixture was stirred for 16 hours and purified by reversed-phase column chromatography (10-80% MeOH in water) to give 0.112 g (77%) of compound 65.

¹H NMR (400 MHz, DMSO) δ 8.90 (s, 1H), 8.17 (t, J=5.7 Hz, 1H), 7.47 (dd, J=8.4, 2.0 Hz, 1H), 7.38 (d, J=1.9 Hz, 1H), 6.99 (td, J=7.6, 1.5 Hz, 1H), 6.91 (dd, J=7.7, 1.5 Hz, 2H), 6.77 (td, J=7.5, 1.3 Hz, 1H), 6.71-6.63 (m, 2H), 3.35-3.25 (m, 4H), 3.17 (d, J=3.0 Hz, 1H), 2.43 (t, J=7.0 Hz, 2H), 2.35 (t, J=5.0 Hz, 4H), 1.39 (s, 9H).

¹³C NMR (101 MHz, DMSO) δ 164.99, 153.85, 144.36, 140.92, 127.79, 127.71, 127.28, 126.32, 125.16, 122.42, 115.91, 115.83, 114.74, 113.60, 78.76, 56.98, 52.53, 36.67, 28.09.

MS (ESI) m/z=455.3 [M+H]⁺.

### N-(2-(methylamino)ethyl)-10H-phenothiazine-3-carboxamide dihydrochloride (66, CPD-033)

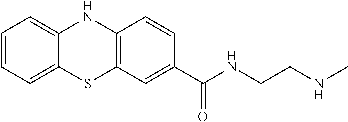 [SMILES: CNCCNC(=O)c1ccc2c(c1)Sc1ccccc1N2]

General procedure E was followed using compound 62 (0.081 g, 0.203 mmol) and 4M HCl in dioxane (0.760 ml, 3.04 mmol) to afford 0.068 g of the desired compound 66 (90%). The reported NMR spectrum refers to dihydrochloride salt.

¹H NMR (400 MHz, DMSO) δ 9.14 (s, 1H), 8.96 (s, 2H), 8.61 (t, J=5.8 Hz, 1H), 7.56 (d, J=8.4 Hz, 1H), 7.48 (s, 1H), 6.98 (t, J=7.6 Hz, 1H), 6.90 (d, J=7.7 Hz, 1H), 6.84-6.69 (m, 3H), 3.51 (q, J=5.8 Hz, 2H), 3.09-2.99 (m, 2H), 2.55 (t, J=5.3 Hz, 3H).

¹³C NMR (101 MHz, DMSO) δ 165.64, 144.62, 140.83, 127.71, 127.51, 127.00, 126.23, 125.44, 122.39, 115.83, 115.73, 114.78, 113.50, 47.97, 35.63, 32.45.

MS (ESI) m/z=300.2 [M+H]⁺.

### N-(2-aminoethyl)-10H-phenothiazine-3-carboxamide dihydrochloride (67, CPD-034)

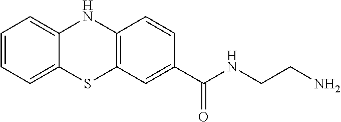 [SMILES: NCCNC(=O)c1ccc2c(c1)Sc1ccccc1N2]

General procedure E was followed using compound 63 (0.063 g, 0.163 mmol) and 4M HCl in dioxane (0.613 ml, 2.45 mmol) to afford 0.054 g of the desired compound 67 (92%). The reported NMR spectrum refers to dihydrochloride salt.

¹H NMR (400 MHz, DMSO) δ 9.16 (s, 1H), 8.59 (t, J=5.5 Hz, 1H), 8.16 (s, 3H), 7.56 (d, J=8.2 Hz, 1H), 7.48 (s, 1H), 6.98 (t, J=7.6 Hz, 1H), 6.90 (d, J=7.5 Hz, 1H), 6.80-6.71 (m, 3H), 3.47 (d, J=6.6 Hz, 3H), 2.95 (p, J=5.7 Hz, 2H).

¹³C NMR (101 MHz, DMSO) δ 165.63, 144.62, 140.87, 127.71, 127.50, 127.05, 126.24, 125.45, 122.38, 115.84, 115.73, 114.79, 113.52, 38.63, 37.05.

MS (ESI) m/z=286.1 [M+H]⁺.

### (10H-phenothiazin-3-yl)(piperazin-1-yl)methanone dihydrochloride (68, CPD-035)

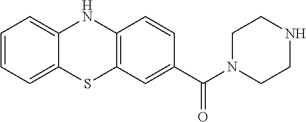 [SMILES: O=C(c1ccc2c(c1)Sc1ccccc1N2)N1CCNCC1]

General procedure E was followed using compound 64 (0.034 g, 0.083 mmol) and 4M HCl in dioxane (0.310 ml, 1.24 mmol) to afford 0.026 g of the desired compound 68 (82%). The reported NMR spectrum refers to dihydrochloride salt.

¹H NMR (400 MHz, DMSO) δ 9.45 (s, 2H), 9.05 (s, 1H), 7.09 (dd, J=8.2, 1.8 Hz, 1H), 7.05-6.95 (m, 2H), 6.91 (d, J=7.5 Hz, 1H), 6.81-6.69 (m, 3H), 3.72-3.65 (m, 4H), 3.13-3.08 (m, 4H).

¹³C NMR (101 MHz, DMSO) δ 168.65, 143.74, 141.17, 127.76, 127.71, 127.43, 126.29, 125.71, 122.33, 116.27, 115.86, 114.71, 113.72, 42.45.

MS (ESI) m/z=312.2 [M+H]⁺.

### N-(2-(piperazin-1-yl)ethyl)-10H-phenothiazine-3-carboxamide trihydrochloride (69, CPD-036)

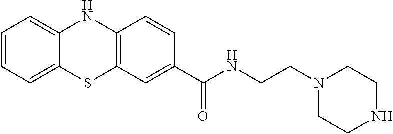 [SMILES: O=C(NCCN1CCNCC1)c1ccc2c(c1)Sc1ccccc1N2]

General procedure E was followed using compound 65 (0.086 g, 0.189 mmol) and 4M HCl in dioxane (0.709 ml, 2.84 mmol) to afford 0.082 g of the desired compound 69 (93%). The reported NMR spectrum refers to trihydrochloride salt.

¹H NMR (400 MHz, DMSO) δ 9.75 (s, 2H), 9.09 (s, 1H), 8.63 (t, J=5.5 Hz, 1H), 7.56 (dd, J=8.3, 2.0 Hz, 1H), 7.48 (d, J=1.9 Hz, 1H), 6.99 (t, J=7.6 Hz, 1H), 6.91 (d, J=7.6 Hz, 1H), 6.81-6.67 (m, 3H), 3.63 (q, J=5.9 Hz, 2H), 3.46-3.38 (m, 8H, overlaps with the solvent), 3.33 (d, J=5.9 Hz, 2H).

¹³C NMR (101 MHz, DMSO) δ 164.74, 143.95, 140.00, 127.00, 126.75, 126.04, 125.51, 124.65, 121.70, 115.07, 115.05, 114.01, 112.77, 54.59, 47.27, 33.05 (1 si.

MS (ESI) m/z=355.2 [M+H]⁺.

### 10H-phenothiazine-3-carbaldehyde (71, CPD-037)

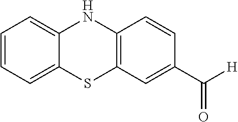 [SMILES: O=Cc1ccc2c(c1)Sc1ccccc1N2]

Compound 71 was synthesized according to the method described in Kumar et al. (2016) J. Mater. Chem. C, 4, 6769-6777. See Scheme 4 above. Yield 31%.

¹H NMR (400 MHz, DMSO) δ 9.64 (s, 1H), 9.25 (s, 1H), 7.49 (dd, J=8.2, 1.8 Hz, 1H), 7.36 (d, J=1.8 Hz, 1H), 7.00 (td, J=7.6, 1.5 Hz, 1H), 6.91 (dd, J=7.7, 1.4 Hz, 1H), 6.80 (td, J=7.5, 1.3 Hz, 1H), 6.76-6.65 (m, 2H).

¹³C NMR (101 MHz, DMSO) δ 189.93, 147.14, 139.59, 130.45, 127.87, 127.40, 126.28, 123.10, 116.68, 115.77, 115.11, 113.97.

MS (ESI) m/z=228.0 [M+H]⁺, 226.0 [M−H]⁻.

### N-((10H-phenothiazin-3-yl)methyl)-2-(piperazin-1-yl)ethan-1-amine (72, CPD-038)

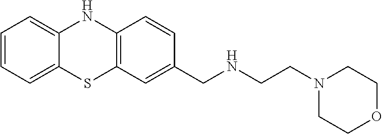 [SMILES: c1ccc2c(c1)Nc1ccc(CNCCN3CCOCC3)cc1S2]

4-(2-Aminoethyl)morpholine (0.078 ml, 0.594 mmol) was dissolved in 5 ml of THF, followed by the addition of acetic acid (0.521 ml, 9.11 mmol) and compound 71 (0.090 g, 0.396 mmol). After 5 minutes, sodium cyanoborohydride (0.062 g, 0.990 mmol) was added and the reaction mixture was stirred at room temperature overnight. After completion, the reaction was quenched with water and the product was extracted twice into DCM. The combined organic layers were dried over sodium sulphate, filtered and loaded on Celite. The crude compound was purified by reversed-phase flash chromatography (10-70% MeOH in water) to yield 0.068 g of compound 72 (50%).

¹H NMR (400 MHz, DMSO) δ 8.75 (s, 1H), 7.10-7.05 (m, 2H), 7.00 (td, J=7.6, 1.5 Hz, 1H), 6.92 (dd, J=7.7, 1.4 Hz, 1H), 6.77 (td, J=7.5, 1.2 Hz, 1H), 6.71-6.65 (m, 2H), 3.97 (s, 2H), 3.59 (t, J=4.5 Hz, 4H), 2.98 (t, J=6.2 Hz, 2H), 2.54-2.33 (m, 6H).

¹³C NMR (101 MHz, DMSO) δ 143.11, 141.89, 130.14, 128.42, 128.25, 126.74, 125.37, 122.62, 117.10, 116.20, 115.07, 114.60, 66.46, 53.94, 53.42, 49.84, 42.88.

MS (ESI) m/z=342.1 [M+H]⁺.

### 1-Chloro-10H-phenothiazine (74, CPD-039)

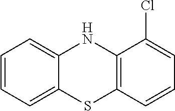 [SMILES: Clc1cccc2c1Nc1ccccc1S2]

Compound 74 was synthesized according to the method described in Kumar et al. (2020) SN Applied Sciences, 2, 1241. See Scheme 5 above. Yield 75%.

¹H NMR (400 MHz, DMSO) δ 7.21 (s, 1H), 6.29 (ddd, J=9.4, 8.0, 1.4 Hz, 2H), 6.18 (td, J=7.7, 1.4 Hz, 1H), 6.14-6.05 (m, 2H), 6.03-5.88 (m, 2H).

¹³C NMR (101 MHz, DMSO) δ 140.96, 138.12, 127.99, 127.67, 126.12, 125.27, 122.95, 122.40, 119.17, 118.31, 116.77, 116.23.

MS (ESI) m/z=232.1, 234.1 [M+H]⁺.

### 2. Biological Evaluation of the Compounds of the Invention

### Fluorescence-Enabled Inhibited Autoxidation (FENIX)

Ref. Shah et al. (2019) Cell Chem. Biol. 26, 1594-1607

Unless otherwise stated, laboratory reagent grade solvents were used. Reagents were obtained from various commercial sources and were used without any prior purification. L-α-phosphatidylcholine (Egg, Chicken), powder was purchased from Merck Life Science B.V.. DTUN and STY-BODIPY were synthesized according to methods described in Shah et al. (2019) Cell Chem. Biol. 26, 1594-1607. Fluorescence was measured using the UV/vis spectrophotometer Synergy MX, Biotek with Gen5.

In order to quantify radical trapping antioxidant activity in phospholipid bilayers to a clear bottom black-walled 96-well plate for fluorescence-base assays (Invitrogen by Thermo Fisher Scientific) was added 250 μL of a solution containing liposomes (1 mM), styrene-conjugated BODIPY (STY-BODIPY, 1 μM), and the respective radical trapping antioxidant (RTA, 2 μM). The solutions were made in larger volumes in Eppendorfs, pre-mixed and 250 μL aliquot was transferred to each well. The plate was incubated for 10 minutes at 37° C. in the BioTek SynergyMx plate reader, followed by a fast-mixing protocol for 5 minutes. The plate was ejected from the plate reader and autoxidation was initiated by the addition of a 50 μL aliquot of (E)-1,2-bis((2-methyldecan-2-yl)oxy)diazene (DTUN, 0.2 mM in EtOH/PBS 3/47, v/v), followed by another mixing protocol for 5 minutes. Data were acquired by excitation probes at 488 nm and emission was measured at 518 nm (read intervals 1.0 min). Kinetic read parameters: (i) optic position: bottom, (ii) gain: 80, (iii) bandwidth: 9.0. The results of the FENIX assay are presented in Table 2 as inhibition rate constants (kᵢₙₕ), logkᵢₙₕ and stoichiometries (n) of tested compounds.

TABLE 2

| | FENIX assay | | |
|---|---|---|---|
| Compound | kᵢₙₕ (10⁴ M⁻¹s⁻¹) ± | | |
| code | SD | Logkᵢₙₕ | Stoichiometry |
| CPD-001 | 7.89 ± 0.45 | 1.34 ± 0.01 | 4.90 ± 0.02 |
| CPD-002 | 3.04 ± 0.05 | 2.11 ± 0.07 | 4.48 ± 0.01 |
| CPD-003 | 3.43 ± 0.05 | 1.89 ± 0.06 | 4.53 ± 0.01 |
| CPD-004 | 3.04 ± 0.07 | 2.37 ± 0.18 | 4.48 ± 0.01 |
| CPD-005 | 7.63 ± 0.55 | 1.53 ± 0.28 | 4.88 ± 0.03 |
| CPD-006 | 6.71 ± 0.23 | 1.61 ± 0.07 | 4.83 ± 0.01 |
| CPD-007 | 5.16 ± 0.38 | 1.82 ± 0.11 | 4.71 ± 0.03 |
| CPD-008 | 5.43 ± 0.39 | 1.68 ± 0.06 | 4.73 ± 0.03 |
| CPD-009 | 5.89 ± 0.45 | 1.60 ± 0.02 | 4.77 ± 0.03 |
| CPD-010 | 12.86 ± 0.90 | 1.80 ± 0.08 | 5.11 ± 0.03 |
| CPD-011 | 1.77 ± 0.22 | 1.77 ± 0.07 | 4.25 ± 0.05 |
| CPD-012 | 2.58 ± 0.45 | 2.02 ± 0.12 | 4.41 ± 0.08 |
| CPD-013 | 2.00 ± 0.22 | 2.04 ± 0.01 | 4.30 ± 0.05 |
| CPD-014 | 2.26 ± 0.05 | 1.61 ± 0.09 | 4.35 ± 0.01 |
| CPD-015 | 6.28 ± 0.35 | 1.88 ± 0.15 | 4.80 ± 0.08 |
| CPD-016 | 4.78 ± 0.17 | 1.96 ± 0.08 | 4.68 ± 0.02 |
| CPD-017 | 2.27 ± 0.03 | 2.03 ± 0.02 | 4.36 ± 0.01 |
| CPD-018 | 2.47 ± 0.32 | 2.10 ± 0.09 | 4.39 ± 0.06 |
| CPD-019 | 4.42 ± 0.22 | 1.91 ± 0.15 | 4.64 ± 0.02 |
| CPD-020 | 3.56 ± 0.07 | 1.97 ± 0.12 | 4.55 ± 0.01 |
| CPD-021 | 2.40 ± 0.02 | 2.06 ± 0.07 | 4.38 ± 0.01 |
| CPD-022 | 1.96 ± 0.30 | 1.74 ± 0.12 | 4.29 ± 0.07 |
| CPD-023 | 3.11 ± 0.29 | 1.80 ± 0.01 | 4.49 ± 0.04 |
| CPD-024 | 3.41 ± 0.21 | 1.95 ± 0.33 | 4.53 ± 0.03 |
| CPD-025 | 3.75 ± 0.50 | 1.78 ± 0.02 | 4.57 ± 0.06 |
| CPD-026 | 2.28 ± 0.24 | 2.11 ± 0.09 | 4.36 ± 0.05 |
| CPD-027 | 2.52 ± 0.43 | 2.13 ± 0.15 | 4.40 ± 0.08 |
| CPD-028 | 2.59 ± 0.21 | 1.82 ± 0.09 | 4.41 ± 0.04 |
| CPD-029 | 2.64 ± 0.15 | 1.97 ± 0.13 | 4.42 ± 0.02 |
| CPD-030 | 4.24 ± 0.05 | 2.13 ± 0.13 | 4.63 ± 0.01 |
| CPD-031 | 1.15 ± 0.01 | 2.11 ± 0.04 | 4.06 ± 0.01 |
| CPD-032 | 1.14 ± 0.01 | 2.29 ± 0.11 | 4.06 ± 0.01 |
| CPD-033 | 1.21 ± 0.01 | 2.68 ± 0.07 | 4.09 ± 0.01 |
| CPD-034 | 1.23 ± 0.01 | 2.41 ± 0.13 | 4.09 ± 0.01 |
| CPD-035 | 1.26 ± 0.04 | 2.51 ± 0.05 | 4.10 ± 0.02 |
| CPD-036 | 1.24 ± 0.04 | 2.30 ± 0.09 | 4.09 ± 0.01 |
| CPD-038 | 1.03 ± 0.01 | 3.77 ± 0.16 | 4.01 ± 0.01 |

### Inhibition of ML162-Induced Ferroptosis in HT1080 Human Fibrosarcoma Cells

Human fibrosarcoma cells HT1080 cells were obtained from American Type Culture Collection (ATCC). HT1080 cells were cultured in EMEM medium supplemented with 10% FCS and L-glutamine (1 mM), sodium pyruvate and nonessential amino acids. Cell death was measured using Envision multimode plate reader (PE). In order to determine IC₅₀ values, HT1080 were seeded in a 384-well plate at a density of 3500 cells/well in 40 μl in an incubator overnight at 37° C. with 5% CO2. The next day, the cells were pretreated for 1 h (in triplicates) with a 1/3 dilution series of ferrostatin-1 analogues ranging from 1 μM to 0.5 nM and Sytox Green (1.6 μM). All the compound's stock solutions were prepared in DMSO at 100 mM concentration. After stimulating the cells with ML-162 1 μM the plate was transferred to the incubator. SytoxGreen intensity was measured after 8, 12, 16 and 24 h using an excitation filter of 485 nm and an emission filter of 535 nm. Dose-response curves were made as % cell death inhibition, taking ML162 treated samples as 0% inhibition and Fer-1 (1 μM)+ML-162 samples as the 100% inhibition. Cell death percentage was calculates as 100−((x−100% inh)/(0% inh−100% inh))*100). Curves were plotted in Spotfire software, and IC₅₀ values were calculated using logistic regression curves. Data is presented in Table 3. Cytotoxicity evaluation

24 hours before the experiment, human fibrosarcoma cells HT-1080 were seeded in a 96-well plate at a density of 10 000 cells/well in α-MEM medium (Gibco, 22571-020) supplemented with 10% heat-inactivated FBS (Gibco; 10270-106), penicillin/streptavidine (Gibco; 15140-122), L-glutamine (Gibco; 25030-081), MEM non-essential amino acid (Sigma; M7145) and 0.4% sodium pyruvate (Sigma; S8636). Afterwards, cells were incubated at 37° C. at 5% CO₂. The next day, test compounds were added to the cell culture medium in a dilution series 1:3 with 500 μM at the highest concentration. All the compound's stock solutions were prepared in DMSO at 100 mM. In addition to the test compounds, Sytox™ Green (1.7 μM) (Invitrogen S7020), a fluorescent dye used to assess cell viability, was added and cells were incubated for 24 hours. Afterwards, cell death was measured using a FLUOstar® Omega microplate reader at an excitation wavelength of 485 nm and emission was measured at 535 nm. Dose-response curve were made as % cell death, taking untreated wells as negative control and Triton X-100 at a concentration of 0.05% was used as a positive control representing 100% cell death. Cell death percentage was calculated using Excel as ((x−0% Negative control)/(positive control−negative control)*100). The processed data were visualized and analysed further using GraphPad Prism 9 software. Data is presented in Table 3.

### Selectivity Index (SI)

The selectivity index (SI) is a ratio that measures the window between cytotoxicity and the inhibitory ferroptotic effect. The higher the SI ratio, the more theoretically effective and safe a drug would be during in vivo treatment. SI values ≥10 are generally considered beneficial in vitro. Data is calculated by following the equation: SI=mean CC₅₀ against normal cells HT-1080/mean IC₅₀ from inhibition of ML162-induced ferroptosis in HT-1080.

### Kinetic Solubility

A turbidimetric method was used. First, a series of DMSO compound stock solutions were prepared (0.15-5 mM) from the stock solution of the compound in DMSO (10 mM). An aliquot of 4 μL stock solution was added to 196 μL PBS buffer (pH 7.4). A series of concentrations were prepared (3.13-200 μM), including a blank on a microtiter plate. The microtiter plate was shaken for 10 seconds and incubated for 2 hours at 37° C. Turbidity was measured using the UV/vis spectrophotometer Synergy MX, Biotek with Gen5. When there was no turbidity measured at a given concentration the sample was assumed to be dissolved. Data is presented in Table 3.

### Central Nervous System Multiparameter Optimization (CNS MPO) Score

In order to evaluate brain penetration, central nervous system multiparameter optimization (CNS MPO) score was calculated using CDD Vault software (Collaborative Drug Discovery Inc., Burlingame, California, USA). CNS MPO score consists of six fundamental physicochemical properties: lipophilicity, calculated partition coefficient (ClogP), calculated distribution coefficient at pH 7.4 (ClogD), molecular weight (MW), topological polar surface area (TPSA), number of hydrogen-bond donors (HBDs), and most basic center (pKₐ). A CNS MPO score of >4.0 is preferable to penetrate the brain. Data is presented in Table 3.

TABLE 3

| | | In vitro | | | |
|---|---|---|---|---|---|
| | Kin. | Ferroptosis | | | CNS |
| Compound | solubility | inhibition IC₅₀ | Cytotoxicity | | MPO |
| code | (μM) | (nM) | CC₅₀ (μM) | SI | score |
| CPD-001 | 100-200 | 5.81 ± 2.69 | — | — | 4.3 |
| CPD-002 | 25-50 | 5.66 ± 1.76 | — | — | 2.7 |
| CPD-003 | 12.5-25 | 12.11 ± 6.27 | — | — | 2.7 |
| CPD-004 | 100-200 | 7.43 | — | — | 2.7 |
| CPD-005 | >200 | 12.4 ± 6.70 | — | — | 4.9 |
| CPD-006 | >200 | 7.07 ± 2.17 | 177.4 | 2509 | 4.9 |
| CPD-007 | >200 | 8.55 ± 5.23 | 170.5 | 1994 | 4.0 |
| CPD-008 | >200 | 11.7 ± 7.39 | 207.8 | 1776 | 4.3 |
| CPD-009 | >200 | 2.40 ± 0.792 | 127.8 | 5325 | 5.0 |
| CPD-010 | >200 | 27.5 ± 11.6 | 308.4 | 1121 | 4.1 |
| CPD-011 | 50-100 | 11.6 ± 3.17 | — | — | 3.2 |
| CPD-012 | 50-100 | 9.18 ± 5.78 | — | — | 3.0 |
| CPD-013 | 12.5-25 | 9.12 ± 5.57 | — | — | 3.0 |
| CPD-014 | 25-50 | 11.9 ± 6.45 | — | — | 2.7 |
| CPD-015 | 12.5-25 | 20.34 | — | — | 2.7 |
| CPD-016 | 12.5-25 | 12.97 | — | — | 3.2 |
| CPD-017 | >200 | 3.71 ± 2.77 | — | — | 2.7 |
| CPD-018 | >200 | 3.96 ± 3.21 | — | — | 2.4 |
| CPD-019 | 100-200 | 4.67 | — | — | 2.5 |
| CPD-020 | >200 | 6.65 | — | — | 3.0 |
| CPD-021 | >200 | 3.75 ± 2.38 | — | — | 3.3 |
| CPD-022 | 100-200 | 5.50 ± 1.66 | — | — | 3.0 |
| CPD-023 | 100-200 | 4.20 ± 3.05 | — | — | 2.7 |
| CPD-024 | 50-100 | 4.46 ± 3.80 | — | — | 2.7 |
| CPD-025 | 50-100 | 6.33 ± 1.28 | — | — | 2.0 |
| CPD-026 | 100-200 | 3.58 ± 2.61 | 26.49 | 740 | 3.5 |
| CPD-027 | 50-100 | 3.65 ± 2.64 | — | — | 3.1 |
| CPD-028 | 100-200 | 6.52 ± 0.798 | — | — | 2.7 |
| CPD-029 | 50-100 | 5.21 ± 0.893 | — | — | 2.4 |
| CPD-030 | 100-200 | 4.69 | — | — | 2.5 |
| CPD-031 | >200 | 110 ± 14.7 | — | — | 5.4 |
| CPD-032 | >200 | 46.7 ± 1.33 | — | — | 5.4 |
| CPD-033 | >200 | 7.32 ± 1.08 | 161.8 | 2210 | 4.2 |
| CPD-034 | >200 | 100 ± 28.1 | — | — | 4.4 |
| CPD-035 | >200 | 10.3 ± 0.79 | 178.6 | 1734 | 5.5 |
| CPD-036 | >200 | 15.5 ± 0.19 | 160.6 | 1036 | 4.3 |
| CPD-037 | 12.5-50 | 28.0 ± 11.2 | — | — | 4.5 |
| CPD-038 | >200 | 11.2 ± 2.80 | — | — | 5.0 |
| CPD-039 | >200 | 60.9 ± 5.64 | — | — | 3.2 |

### Human and Mouse Microsomal Stability

### Experimental Procedure

Pooled liver microsomes (Ultrapool Human Liver microsomes or Mouse CD-1 male Liver microsomes), with a protein concentration of 20 mg/ml, were purchased from Corning Gentest™ (now Discovery Life Science-Gentest) and stored at −80° C. until use. The microsomes (final protein concentration 0.5 mg/ml), 0.1 M phosphate buffer pH 7.4 and test compound (final substance concentration 1 μM), were preincubated at 37° C. for 10 min before the addition of NADPH Regenerating System (solution A and B from Corning Gentest™, final concentration 1 mM) to initiate the reaction. For each compound tested, a control without the cofactor was included. In this case, 0.1 M phosphate buffer at pH 7.4 was added instead of the NADPH regenerating system. In addition, with each new batch of microsomes, two positive control compounds were incorporated. These controls represented substances with high (verapamil, with human Clᵢₙₜ=178.9 μL/min*mg; diazepam, with mouse Clᵢₙₜ=549 μL/min*mg) and low clearance (dextromethorphan, with human Clᵢₙₜ=34.6 μL/min*mg; diphenhydramine, with mouse Clᵢₙₜ=64.0 μL/min*mg).

Each compound was incubated for 45 minutes at 37° C. and shaken at 500 rpm. The reactions were stopped by transferring the incubate into acetonitrile, containing an internal standard, in Eppendorfs at the appropriate time points in a 1:3 ratio (0, 5, 10, 15, 30, and 45 minutes for compounds, and 0, 15, and 45 minutes for the negative control of those compounds). Subsequently, the Eppendorfs were centrifuged at 3000 rpm for 20 minutes at 4° C. to precipitate the protein.

### Quantitative Analysis

After protein precipitation, the sample supernatant from each time point was analyzed using UPLC-MS/MS. The analysis followed the general UPLC method for microsomal stability described below. The UPLC (ultra-performance liquid chromatography), used to quantify the microsomal stability of the products, was an ACQUITY UPLC H-Class system with a TUV detector Waters (not used in this assay) coupled to an MS/MS detector Xevo Waters TQD. Waters Acquity UPLC BEH C18 1.7 μm, 2.1 mm×50 mm column was used. The eluent was composed of two different solvents. Solvent A consisted of water with 0.1% formic acid, solvent B was acetonitrile with 0.1% formic acid. The column was first equilibrated for with a mixture of 95% solvent A and 5% solvent B until the delta of psi decrease below 40 psi. The method began with an a short equilibration to reach 50% of solvent A and B in 0.15 min. Following this, solvent B was increased linearly to 95% over 2.75 min before being held constant for 0.70 min (flow rate 0.7 ml/min). The specific masses of the compounds were tracked using tuning files generated via Intellistart®, and quantification was aided by an internal standard.

### Data Processing

All obtained readouts were processed using Microsoft Excel. From a plot of In peak area ratio (compound peak area/internal standard peak area) against time, the gradient of a line is determined by linear regression. Subsequently several parameters were calculated using the equations below:

$$Elimination rate constant ( k ) = ( - gradient )$$

$$Half Time ( {t}_{1 / 2} ) = ln ( 2 ) / k$$

$${Cl}_{i n t} ( μL / min / mg of protein ) = V \times ln ( 2 ) \times {t}_{1 / 2}$$

Where V=(Incubation volume μL)/(Microsomal protein mg/g liver) Data is presented in Table 4.

### Log D Shake Flask Assay

Lipophilicity affects drugs' distribution in tissues, absorption, binding traits, and is an important factor in determining solubility. The log D (distribution coefficient) quantifies lipophilicity, often measured by assessing a compound's preference for an organic solvent (like octanol) versus an aqueous buffer.

### Experimental Procedure

A total of 40 μL of a 10 mM stock solution in DMSO was added to 1.960 mL of a mixture of octanol and phosphate buffer saline (PBS), pH 7.4 (v/v, 1/1). Octanol was first saturated with PBS, and PBS was first saturated with octanol before the two solvents were mixed. After two hours of shaking at room temperature, the two layers were separated. A total of 10 μL of each layer was added to 390 μL of CH₃OH before the sample was analyzed with LC/MS/MS. The experiment was done in duplicate. The logD is calculated from the formula:

$$logD = log [ ( \frac{{Conc}_{INITIAL} - {Conc}_{FINAL}}{{Conc}_{FINAL}} ) \times ( \frac{{V}_{aq}}{{V}_{oct}} ) ]$$

An optimal range for lipophilicity tends to be if the compound has a log 0 value between 0 and 3. Typically, these compounds have a good balance between solubility and permeability and this range tends to be optimal for oral absorption and cell membrane permeation.

The optimal log 0 for blood-brain barrier permeation is approximately 2. Hydrophilic compounds (log 0<0) typically are highly soluble but exhibit low permeability across the gastrointestinal tract or blood-brain barrier. Highly lipophilic compounds (log 0>5) exhibit problems with metabolic instability, high plasma protein binding and low solubility which leads to variable and poor oral absorption. Data is presented in Table 4.

TABLE 4

| | Microsomal | | Microsomal | | |
|---|---|---|---|---|---|
| | stability | | stability | | |
| | in mouse | | in human | | |
| | | Cliₙₜ in liver | | Clᵢₙₜ in liver | |
| | t_{1/2} | microsomes | t_{1/2} | microsomes | |
| | (min) | (μL/min*mg) | (min) | (μL/min*mg) | |
| Compound code | a | b | a | b | LogD |
| Diazepam | 2.0 | 707.0 | n.d | n.d | — |
| Diphenhydramine | 30.7 | 45.1 | n.d | n.d | — |
| Verapamil | nd. | nd. | 7.7 | 178.9 | — |
| Dextro- | nd. | nd. | 40.0 | 34.6 | — |
| methorphan | | | | | |
| CPD-005 | 5.4 | 254.6 | nd. | n.d | n.d |
| CPD-006 | 37.2 | 37.2 | 202.6 | 6.8 | 2.55 |
| CPD-007 | 88.4 | 21.1 | 73.8 | 18.8 | 1.1 |
| CPD-008 | >250 | <5.5 | >250 | <5.5 | 0.9 |
| CPD-009 | 51.6 | 26.8 | 62.8 | 22.1 | 1.55 |
| CPD-010 | >250 | <5.5 | 84.7 | 16.4 | 1.1 |
| CPD-026 | 64.2 | 21.6 | >250 | <5.5 | 4.0 |
| CPD-033 | 29.2 | 47.5 | 89.0 | 15.6 | n.d |
| CPD-035 | 27.4 | 50.6 | 75.3 | 18.4 | 2.0 |
| CPD-036 | 93.6 | 14.8 | >250 | <5.5 | 1.5 |
| CPD-038 | 7.9 | 174.9 | nd. | nd. | n.d |

a Metabolism determined in Corning ultra pool human liver microsome (HLM) or Mouse CD-1 liver microsome (MLM) activated with NAPDH and expressed as half life (t_{1/2} in min); b The Clᵢₙₜ (liver microsomes) was calculated using the measured microsomal t_{1/2} and considers several experimental variables such as the protein concentration and the volume of incubation. Diazepam, diphenhydramine, verapamil and dextromethorphan were used as controls.

1. A compound of formula (I) or a stereoisomer or tautomer thereof, 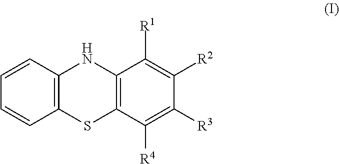
wherein,
R¹ is selected from hydrogen, amino, —NR⁶R⁷, halo, —C(O)NH(CR⁸R⁹)ₜR¹⁰, —C(O)R¹¹, —CR¹²R¹³NH(CR¹⁴R¹⁵)_{w}R¹⁶, —COOR¹¹, —S(O)₂R¹¹, —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z¹;
wherein t is an integer selected from 0, 1, 2, 3 or 4;
wherein w is an integer selected from 0, 1, 2, 3 or 4;
R² is selected from hydrogen, amino, —NR⁶R⁷, halo, C(O)NH(CR⁸R⁹)ₙR¹⁰, —C(O)R¹¹, —CR¹²R¹³NH(CR¹⁴R¹⁵)ₘR¹⁶, —COOR¹¹, —S(O)₂R¹¹, —SO₂NR⁶R⁷, cycloalkyl, aryl, heterocyclyl, and heteroaryl; wherein said cycloalkyl, aryl, heterocyclyl, or heteroaryl can be unsubstituted or substituted with one or more Z²;
wherein n is an integer selected from 0, 1, 2, 3 or 4;
wherein m is an integer selected from 0, 1, 2, 3 or 4;
R³ is selected from hydrogen, amino, —NR⁶R⁷, halo, NO₂, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z³;
wherein p is an integer selected from 0, 1, 2, 3 or 4;
wherein q is an integer selected from 0, 1, 2, 3 or 4;
R⁴ is selected from hydrogen, amino, —NR⁶R⁷, halo, NO₂, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, cycloalkyl, aryl, arylalkyl, heterocyclyl, heterocyclylalkyl, heteroaryl and heteroarylalkyl; wherein said cycloalkyl, aryl, arylalkyl, heterocyclyl, heterocyclylalkyl, heterocyclylalkyl, heteroaryl or heteroarylalkyl can be unsubstituted or substituted with one or more Z⁴;
wherein y is an integer selected from 0, 1, 2, 3 or 4;
wherein z is an integer selected from 0, 1, 2, 3 or 4;
wherein at least one of R¹ to R⁴ is not hydrogen;
each R⁶ and R⁷ is independently selected from hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1a};
each R⁸, R⁹, R¹², R¹³, R¹⁴ and R¹⁵ is selected from hydrogen, halogen, hydroxyl, amino, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one or more Z^{1b};
each R¹⁰, R¹¹ and R¹⁶ is selected from heterocyclyl, —NR⁶R⁷, alkyl, cycloalkyl, aryl, arylalkyl, heterocyclyl, heterocyclylalkyl, heteroaryl, and heteroarylalkyl; wherein said heterocyclyl, alkyl, cycloalkyl, aryl, arylalkyl, heterocyclylalkyl, heteroaryl or heteroarylalkyl can be unsubstituted or substituted with one or more Z^{1c};
each R¹⁷ is independently selected from to alkyl, aryl, cycloalkyl, arylalkyl, heterocyclyl, heteroaryl;
each R¹⁸ and R¹⁹ is independently selected from hydrogen, alkyl, aryl, cycloalkyl, arylalkyl, heterocyclyl, heteroaryl;
each Z¹, Z², Z³, Z⁴, Z^{1a}, and Z^{1b} is independently selected from halo, haloalkyl, alkyl, haloalkyloxy, cycloalkyl, aryl, alkylaryl, heterocyclyl, heteroaryl, hydroxyl, —OR¹⁷, cyano, amino, —NR¹⁷R¹⁸, —C(O)₂R¹⁸, —C(O)NR¹⁸R¹⁹, —C(O)R¹⁷, —S(O)R¹⁸, —S(O)₂R¹⁸, —S(O)₂NR¹⁸R¹⁹, nitro;
each Z^{1c} is independently selected from u halo, haloalkyl, alkyl, haloalkyloxy, cycloalkyl, aryl, alkylaryl, heterocyclyl, heteroaryl, hydroxyl, —OR¹⁷, cyano, amino, —NR¹⁷R¹⁸, —C(O)₂R¹⁸, —C(O)NR¹⁸R¹⁹, —S(O)R¹⁸, —S(O)₂R¹⁸, —S(O)₂NR¹⁸R¹⁹ nitro;
wherein when R¹ is 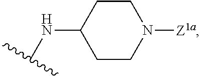
then R³ is not H, Cl or —C(O)R¹¹;
or a solvate, hydrate, pharmaceutically acceptable salt, or prodrug thereof;
with the proviso that said compound is not

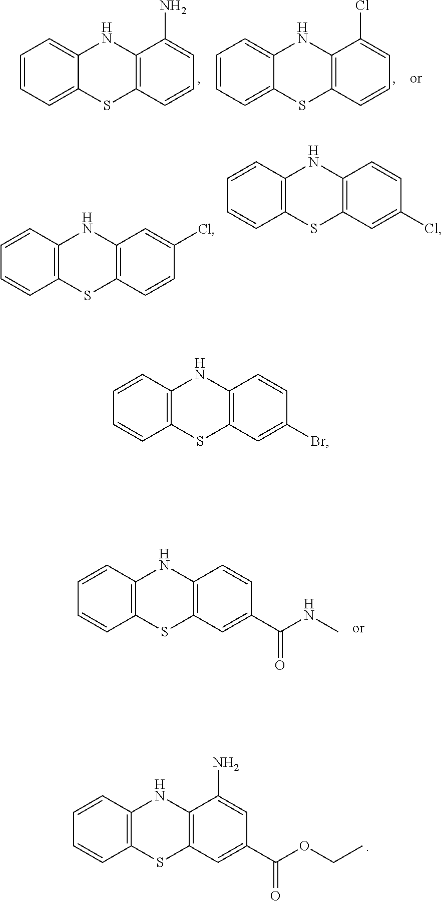

2. The compound according to claim 1, having structural formula (IA):

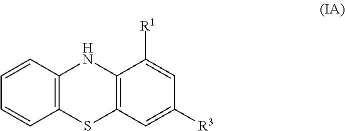

3. The compound according to claim 1, having structural formula (IIA), (IIB) (IIC) or (IID):

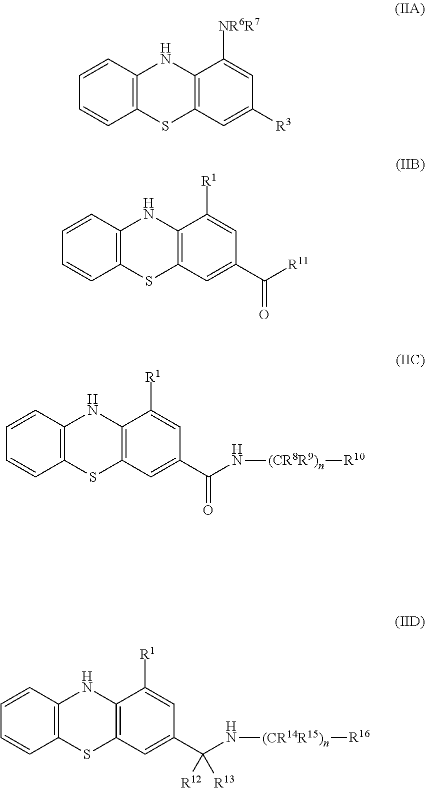

4. The compound according to claim 1, wherein, R¹ is selected from hydrogen, amino, —NR⁶R⁷, halo, —C(O)NH(CR⁸R⁹)ₜR¹⁰, —C(O)R¹¹, —CR¹²R¹³NH(CR¹⁴R¹⁵)_{w}R¹⁶ aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z¹;
wherein t is an integer selected from 1, 2, or 3;
wherein w is an integer selected from 1, 2, or 3;
R² is selected from hydrogen, amino, —NR⁶R⁷, halo, NO₂, —C(O)NH(CR⁸R⁹)R¹⁰, —C(O)R¹, —CR¹²R¹³NH(CR¹⁴R¹⁵)ₘR¹⁶, aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z²;
wherein n is an integer selected from 1, 2, or 3;
wherein m is an integer selected from 1, 2, or 3;
R³ is selected from hydrogen, amino, —NR⁶R⁷, halo, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, NO₂, —NR⁶R⁷, halo, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z⁴;
wherein y is an integer selected from 1, 2, or 3;
wherein z is an integer selected from 1, 2, or 3
wherein at least one of R¹ to R⁴ is not hydrogen;
each R⁶ and R⁷ is independently selected from hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one, two or three Z^{1a};
each R⁸, R⁹, R¹², R¹³, R¹⁴ and R¹⁵ is selected from hydrogen, halogen, hydroxyl, amino, alkyl, cycloalkyl, and aryl;
each R¹⁰, R¹¹ and R¹⁶ is selected from heterocyclyl, —NR⁶R⁷, alkyl, arylalkyl, heterocyclylalkyl, and heteroarylalkyl; wherein said heterocyclyl, alkyl, arylalkyl, heterocyclylalkyl, or heteroarylalkyl can be unsubstituted or substituted with one, two or three Z^{1c}.

5. The compound according to claim 1, wherein,
R¹ is selected from hydrogen, amino, —NR⁶R⁷, choro, bromo iodo, —C(O)NH(CH₂)₂R¹⁰, —C(O)R¹¹, —CR¹²R¹³NH(CH₂)₂R¹⁶, C₆₋₁₂aryl, heterocyclyl, and heteroaryl;
wherein said C₆₋₁₂aryl, heterocyclyl, or heteroaryl can be unsubstituted or substituted with one, two or three Z¹;
R² is selected from hydrogen, amino, —NR⁶R⁷, halo, NO₂, —C(O)NH(CH₂)₂R¹⁰, —C(O)R¹¹, —CR¹²R¹³NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z²;
R³ is selected from hydrogen, amino, —NR⁶R⁷, halo, —C(O)NH(CR⁸R⁹)_{q}R¹, —C(O)R¹¹, —CR¹²R¹³NH(CR¹⁴R¹⁵)ₚR¹⁶, —COOR¹¹, —S(O)₂R¹¹, and —SO₂NR⁶R⁷, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₆₋₁₂aryl, C₆₋₁₂arylC₁₋₆alkyl, heterocyclyl, heterocyclylC₁₋₆alkyl, heteroaryl and heteroarylC₁₋₆alkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, —NR⁶R⁷, chloro, bromo, iodo, NO₂, —C(O)NH(CH₂)₂R¹⁰, —C(O)R¹¹, —CR¹²R¹³NH(CH₂)₂R¹⁶, —COOR¹¹, —S(O)₂R¹¹, —SO₂NR⁶R⁷, C₆₋₁₂aryl, heterocyclyl, and heteroaryl; wherein said C₆₋₁₂aryl, heterocyclyl, or heteroaryl can be unsubstituted or substituted with one, two or three Z⁴;
each R⁶ and R⁷ is independently selected from hydrogen, C₁₋₄alkyl, C₃₋₁₀cycloalkyl, C₆₋₁₂aryl, C₆₋₁₂arylC₁₋₆alkyl, heterocyclyl, heterocyclylC₁₋₄alkyl, heteroaryl, and heteroarylC₁₋₄alkyl; wherein said C₁₋₄alkyl, C₃₋₁₀cycloalkyl, C₆₋₁₂aryl, C₆₋₁₂arylC₁₋₄alkyl, heterocyclyl, heterocyclylC₁₋₄alkyl, heteroaryl or heteroarylC₁₋₄alkyl can be unsubstituted or substituted with one, two or three Z^{1a};
each R⁸, R⁹, R¹², R¹³, R¹⁴ and R¹⁵ is selected from hydrogen, halogen, hydroxyl, amino, C₁₋₄alkyl, C₃₋₁₀cycloalkyl, and C₆₋₁₂aryl;
each R¹⁰, R¹¹ and R¹⁶ is selected from heterocyclyl, —NR⁶R⁷, C₁₋₄alkyl, C₆₋₁₂arylC₁₋₄alkyl, heterocyclylC₁₋₄alkyl, and heteroarylC₁₋₄alkyl; wherein said heterocyclyl, C₁₋₄alkyl, C₆₋₁₂arylC₁₋₄alkyl, heterocyclylC₁₋₄alkyl, and heteroarylC₁₋₄alkyl can be unsubstituted or substituted with one, two or three Z^{1c}.

6. The compound according to claim 1, wherein,
each Z¹, Z², Z³, Z⁴, Z^{1a}, and Z^{1b} is independently selected from halo, haloC₁₋₄akyl, C₁₋₆alkyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1c} is independently selected from halo, haloC₁₋₄akyl, C₁₋₆alkyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, —S(O)H, C₁₋₄akylsulfinyl, —S(O)₂H, C₁₋₄akylsulfonyl, —SO₂NH₂, mono-C₁₋₄akylaminosulfonyl, nitro.

7. The compound according to claim 1, wherein,
R¹ is selected from hydrogen, amino, —NR⁶R⁷, choro, bromo iodo, —C(O)NH(CH₂)₂R¹⁰, —C(O)R¹¹, —CH₂NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z¹;
R² is selected from hydrogen, amino, —NR⁶R⁷, halo, NO₂, —C(O)NH(CH₂)₂R¹⁰, —C(O)R¹¹, —CH₂NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z²;
R³ is selected from hydrogen, amino, —NR⁶R⁷, halo, —C(O)NH(CH₂)_{q}R¹⁰, —C(O)R¹¹, CR¹²R¹³NH(CH₂)ₚR¹⁶, —COOR¹¹, —S(O)₂R¹¹, and —SO₂NR⁶R⁷, C₆₋₁₂aryl, C₆₋₁₂arylC₁₋₆alkyl, heterocyclylC₁₋₆alkyl, and heteroarylC₁₋₆alkyl; wherein said C₆₋₁₂aryl, C₆₋₁₂arylC₁₋₆alkyl, heterocyclylC₁₋₆alkyl, or heteroarylC₁₋₆alkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, —NR⁶R⁷, chloro, bromo, iodo, NO₂, —C(O)NH(CH₂)₂R¹⁰, —C(O)R¹¹, —CH₂NH(CH₂)₂R¹⁶, —COOR¹¹, —S(O)₂R¹¹, —SO₂NR⁶R⁷, C₆₋₁₂aryl, heterocyclyl, and heteroaryl; wherein said C₆₋₁₂aryl, heterocyclyl, or heteroaryl can be unsubstituted or substituted with one, two or three Z⁴;
each R⁶ is H;
each R⁷ is independently selected from hydrogen, C₁₋₄alkyl, C₃₋₁₀cycloalkyl, C₆₋₁₂aryl, C₆₋₁₂arylC₁₋₆alkyl, heterocyclyl, heterocyclylC₁₋₄alkyl, heteroaryl, and heteroarylC₁₋₄alkyl; wherein said C₁₋₄alkyl, C₃₋₁₀cycloalkyl, C₆₋₁₂aryl, C₆₋₁₂arylC₁₋₄alkyl, heterocyclyl, heterocyclylC₁₋₄alkyl, heteroaryl or heteroarylC₁₋₄alkyl can be unsubstituted or substituted with one, two or three Z^{1a};
each R¹² and R¹³ is selected from halogen, hydroxyl, and C₁₋₄alkyl;
each R¹⁰, R¹¹ and R¹⁶ is selected from heterocyclyl, —NR⁶R⁷, C₁₋₄alkyl, C₆₋₁₂arylC₁₋₄alkyl, heterocyclylC₁₋₄alkyl, and heteroarylC₁₋₄alkyl; wherein said heterocyclyl, C₁₋₄alkyl, C₆₋₁₂arylC₁₋₄alkyl, heterocyclylC₁₋₄alkyl, and heteroarylC₁₋₄alkyl can be unsubstituted or substituted with one, two or three Z^{1c}.

8. A pharmaceutical composition comprising a compound of formula (I) according to claim 1 and a pharmaceutically acceptable carrier.

9. A compound of formula (I) according to claim 1, or a pharmaceutical composition according to claim 8, or a compound selected from 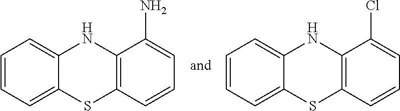 [SMILES: Nc1cccc2c1Nc1ccccc1S2 ; Clc1cccc2c1Nc1ccccc1S2]
for use as a medicament.

10. A compound of formula (I) according to claim 1, or a pharmaceutical composition according to claim 8, or a compound selected from 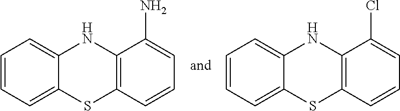 [SMILES: Nc1cccc2c1Nc1ccccc1S2 ; Clc1cccc2c1Nc1ccccc1S2]
for use in the prevention or treatment of a disease associated with ferroptosis and/or oxytosis.

11. The compound for use according to claim 10, wherein the disease associated with ferroptosis and/or oxytosis is selected from liver disease, chronic kidney disease, ocular surface diseases, wound healing, multiple organ dysfunction syndrome, neurological disease, acute renal failure, ischemia-reperfusion injury, sepsis, iron toxicity, prevention of transplant rejection, and iron metabolism-related disease.

12. The compound for use according to claim 11, wherein the liver disease is selected from hemochromatosis, primary biliary cholangitis, non-alcoholic steatohepatitis or liver fibrosis.

13. The compound for use according to claim 11, wherein the neurological disease is selected from Alzheimer's Disease, Parkinson's Disease, Amyotrophic lateral sclerosis, Multiple Sclerosis, Huntington's Disease, Dementia with Lewy bodies, Friedreich's ataxia, multiple sclerosis, stroke, periventricular leukomalacia, intracerebral haemorrhage, frontotemporal dementia, neurodegeneration with brain iron accumulation, or traumatic brain injury.

14. The compound for use according to claim 11, wherein the ischemia-reperfusion injury is selected from myocardial ischemia-reperfusion injury, liver ischemia-reperfusion injury, or renal reperfusion injury.

15. The compound for use according to claim 11, wherein the iron metabolism-related disease is selected from atherosclerosis or diabetes.

16. The compound according to claim 3, wherein,
R¹ is selected from hydrogen, amino, —NR⁶R⁷, halo, —C(O)NH(CR⁸R⁹)R¹⁰, —C(O)R¹¹, —CR¹²R¹³NH(CR¹⁴R¹⁵)_{w}R¹⁶, aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z¹;
wherein t is an integer selected from 1, 2, or 3;
wherein w is an integer selected from 1, 2, or 3;
R² is selected from hydrogen, amino, —NR⁶R⁷, halo, NO₂, —C(O)NH(CR⁸R⁹)˜R¹⁰, —C(O)R¹¹, —CR¹²R¹³NH(CR¹⁴R¹⁵)ₘR¹⁶, aryl, heterocyclyl, and heteroaryl; wherein said aryl, heterocyclyl, heterocyclylalkyl, heterocyclylalkyl, heteroaryl or heteroarylalkyl can be unsubstituted or substituted with one, two or three Z²;
wherein n is an integer selected from 1, 2, or 3;
wherein m is an integer selected from 1, 2, or 3;
R³ is selected from hydrogen, amino, —NR⁶R⁷, halo, —C(O)NH(CR⁸R⁹)ₚR¹⁰, —C(O)R¹¹, —CR¹²R¹³NH(CR¹⁴R¹⁵)_{q}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, NO₂, —NR⁶R⁷, halo, —C(O)NH(CR⁸R⁹)_{y}R¹⁰, —C(O)R¹¹, —CR¹²R¹³NH(CR¹⁴R¹⁵)_{z}R¹⁶, —COOR¹¹, —S(O)₂R¹¹, and —SO₂NR⁶R⁷, aryl, arylalkyl, heterocyclyl, heterocyclylalkyl, heteroaryl and heteroarylalkyl; wherein said aryl, arylalkyl, heterocyclyl, heterocyclylalkyl, heterocyclylalkyl, heteroaryl or heteroarylalkyl can be unsubstituted or substituted with one, two or three Z⁴;
wherein y is an integer selected from 1, 2, or 3;
wherein z is an integer selected from 1, 2, or 3
wherein at least one of R¹ to R⁴ is not hydrogen;
each R⁶ and R⁷ is independently selected from hydrogen, alkyl, cycloalkyl, aryl, arylalkyl, heterocyclyl, heterocyclylalkyl, heteroaryl, and heteroarylalkyl; wherein said alkyl, cycloalkyl, aryl, arylalkyl, heterocyclyl, heterocyclylalkyl, heteroaryl or heteroarylalkyl can be unsubstituted or substituted with one, two or three Z^{1a};
each R⁸, R⁹, R¹², R¹³, R¹⁴ and R¹⁵ is selected from hydrogen, halogen, hydroxyl, amino, alkyl, cycloalkyl, and aryl;
each R¹⁰, R¹¹ and R¹⁶ is selected from heterocyclyl, —NR⁶R⁷, alkyl, arylalkyl, heterocyclylalkyl, and heteroarylalkyl; wherein said heterocyclyl, alkyl, arylalkyl, heterocyclylalkyl, or heteroarylalkyl can be unsubstituted or substituted with one, two or three Z^{1c}.

17. The compound according to claim 3, wherein,
R¹ is selected from hydrogen, amino, —NR⁶R⁷, choro, bromo iodo, —C(O)NH(CH₂)₂R¹⁰, —C(O)R¹¹, —CR¹²R¹³NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z¹;
R² is selected from hydrogen, amino, —NR⁶R⁷, halo, NO₂, —C(O)NH(CH₂)₂R¹⁰, —C(O)R¹¹, —CR¹²R¹³NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z²;
R³ is selected from hydrogen, amino, —NR⁶R⁷, halo, —C(O)NH(CR⁸R⁹)_{q}R¹⁰, —C(O)R¹¹, —CR¹²R¹³NH(CR¹⁴R¹⁵)ₚR¹⁶, —COOR¹¹, —S(O)₂R¹¹, and —SO₂NR⁶R⁷, C₆₋₁₂aryl, C₆₋₁₂arylC₁₋₆alkyl, heterocyclyl, heterocyclylC₁₋₆alkyl, heteroaryl and heteroarylC₁₋₆alkyl; wherein said C₆₋₁₂aryl, C₆₋₁₂arylC₁₋₆alkyl, heterocyclyl, heterocyclylC₁₋₆alkyl, heteroaryl and heteroarylC₁₋₆alkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, —NR⁶R⁷, chloro, bromo, iodo, NO₂, —C(O)NH(CH₂)₂R¹⁰, —C(O)R¹¹, —CR¹²R¹³NH(CH₂)₂R¹⁶, —COOR¹¹, —S(O)₂R¹¹, —SO₂NR⁶R⁷, C₆₋₁₂aryl, heterocyclyl, and heteroaryl; wherein said C₆₋₁₂aryl, heterocyclyl, or heteroaryl can be unsubstituted or substituted with one, two or three Z⁴;
each R⁶ and R⁷ is independently selected from hydrogen, C₁₋₄alkyl, C₃₋₁₀cycloalkyl, C₆₋₁₂aryl, C₆₋₁₂arylC₁₋₆alkyl, heterocyclyl, heterocyclylC₁₋₄alkyl, heteroaryl, and heteroarylC₁₋₄alkyl;
wherein said C₁₋₄alkyl, C₃₋₁₀cycloalkyl, C₆₋₁₂aryl, C₆₋₁₂arylC₁₋₄alkyl, heterocyclyl, heterocyclylC₁₋₄alkyl, heteroaryl or heteroarylC₁₋₄alkyl can be unsubstituted or substituted with one, two or three Z^{1a};
each R⁸, R⁹, R¹², R¹³, R¹⁴ and R¹⁵ is selected from hydrogen, halogen, hydroxyl, amino, C₁₋₄alkyl, C₃₋₁₀cycloalkyl, and C₆₋₁₂aryl;
each R¹⁰, R¹¹ and R¹⁶ is selected from heterocyclyl, —NR⁶R⁷, C₁₋₄alkyl, C₆₋₁₂arylC₁₋₄alkyl, heterocyclylC₁₋₄alkyl, and heteroarylC₁₋₄alkyl; wherein said heterocyclyl, C₁₋₄alkyl, C₆₋₁₂arylC₁₋₄alkyl, heterocyclylC₁₋₄alkyl, and heteroarylC₁₋₄alkyl can be unsubstituted or substituted with one, two or three Z^{1c}.

18. The compound according to claim 3, wherein,
each Z¹, Z², Z³, Z⁴, Z^{1a}, and Z^{1b} is independently selected from halo, haloC₁₋₄akyl, C₁₋₆alkyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, aminocarbonyl, mono-C₁₋₄akylaminocarbonyl, mono-C₃₋₁₂cycloakylaminocarbonyl, C₁₋₄akylcarbonyl, C₃₋₁₂cycloakylcarbonyl, C₆₋₁₂arylcarbonyl, —S(O)H, C₁₋₄akylsulfinyl, —S(O)₂H, C₁₋₄akylsulfonyl, —SO₂NH₂, mono-C₁₋₄akylaminosulfonyl, nitro;
each Z^{1c} is independently selected from halo, haloC₁₋₄akyl, C₁₋₆alkyl, haloC₁₋₄akyloxy, C₃₋₁₂cycloalkyl, C₆₋₁₂aryl, C₆₋₁₂arylC₁₋₆alkyl, heterocyclyl, heteroaryl, hydroxyl, C₁₋₆alkyloxy, C₃₋₁₂cycloalkyloxy, C₆₋₁₂aryloxy, heterocyclyloxy, heteroaryloxy, cyano, amino, mono-C₁₋₄akylamino, mono-C₃₋₁₂cycloakylamino, mono-C₆₋₁₂arylamino, hydroxycarbonyl, C₁₋₄akyloxycarbonyl, C₃₋₁₂cycloakyloxycarbonyl, C₆₋₁₂aryloxycarbonyl, —S(O)H, C₁₋₄akylsulfinyl, —S(O)₂H, C₁₋₄akylsulfonyl, —SO₂NH₂, mono-C₁₋₄akylaminosulfonyl, nitro.

19. The compound according to claim 3, wherein,
R¹ is selected from hydrogen, amino, —NR⁶R⁷, choro, bromo iodo, —C(O)NH(CH₂)₂R¹⁰, —C(O)R¹¹, —CH₂NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z¹;
R² is selected from hydrogen, amino, —NR⁶R⁷, halo, NO₂, —C(O)NH(CH₂)₂R¹⁰, —C(O)R¹¹, —CH₂NH(CH₂)₂R¹⁶, C₆₋₁₂aryl, heterocyclyl, and heteroaryl; wherein said C₆₋₁₂aryl, heterocyclyl, or heteroaryl can be unsubstituted or substituted with one, two or three Z²;
R³ is selected from hydrogen, amino, —NR⁶R⁷, halo, —C(O)NH(CH₂)_{q}R¹⁰, —C(O)R¹¹, —CR¹²R¹³NH(CH₂)ₚR¹⁶, —COOR¹¹, —S(O)₂R¹¹, and —SO₂NR⁶R⁷, C₆₋₁₂aryl, C₆₋₁₂arylC₁₋₆alkyl, heterocyclylC₁₋₆alkyl, and heteroarylC₁₋₆alkyl; wherein said C₆₋₁₂aryl, C₆₋₁₂arylC₁₋₆alkyl, heterocyclylC₁₋₆alkyl, or heteroarylC₁₋₆alkyl can be unsubstituted or substituted with one, two or three Z³;
wherein p is an integer selected from 1, 2, or 3;
wherein q is an integer selected from 1, 2, or 3;
R⁴ is selected from hydrogen, —NR⁶R⁷, amino, —NR⁶R⁷, chloro, bromo, iodo, NO₂, —C(O)NH(CH₂)₂R¹⁰, —C(O)R¹¹, —CH₂NH(CH₂)₂R¹⁶, —COOR¹¹, —S(O)₂R¹¹, —SO₂NR⁶R⁷, C₆₋₁₂aryl, heterocyclyl, and heteroaryl; wherein said C₆₋₁₂aryl, heterocyclyl, or heteroaryl can be unsubstituted or substituted with one, two or three Z⁴;
each R⁶ is H;
each R⁷ is independently selected from hydrogen, C₁₋₄alkyl, C₃₋₁₀cycloalkyl, C₆₋₁₂aryl, C₆₋₁₂arylC₁₋₆alkyl, heterocyclyl, heterocyclylC₁₋₄alkyl, heteroaryl, and heteroarylC₁₋₄alkyl;
wherein said C₁₋₄alkyl, C₃₋₁₀cycloalkyl, C₆₋₁₂aryl, C₆₋₁₂arylC₁₋₄alkyl, heterocyclyl, heterocyclylC₁₋₄alkyl, heteroaryl or heteroarylC₁₋₄alkyl can be unsubstituted or substituted with one, two or three Z^{1a};
each R¹² and R¹³ is selected from hydrogen, halogen, hydroxyl, and C₁₋₄alkyl;
each R¹⁰, R¹¹ and R¹⁶ is selected from heterocyclyl, —NR⁶R⁷, C₁₋₄alkyl, C₆₋₁₂arylC₁₋₄alkyl, heterocyclylC₁₋₄alkyl, and heteroarylC₁₋₄alkyl; wherein said heterocyclyl, C₁₋₄alkyl, C₆₋₁₂arylC₁₋₄alkyl, heterocyclylC₁₋₄alkyl, and heteroarylC₁₋₄alkyl can be unsubstituted or substituted with one, two or three Z^{1c}.
